(19) United States
(12) Patent Application Publication
(10) Pub. No.: US 2017/0162801 A1
(43) Pub. Date: Jun. 8, 2017

(54) ELECTRON BUFFERING MATERIAL AND ORGANIC ELECTROLUMINESCENT DEVICE

(71) Applicant: Rohm and Haas Electronic Materials Korea Ltd., Cheonan (KR)

(72) Inventors: Young-Jun Cho, Seongnam (KR); Kyung-Hoon Choi, Hwaseong (KR); Sang-Hee Cho, Suwon (KR); Jae-Hoon Shim, Seoul (KR); Hong-Yeop Na, Seoul (KR)

(21) Appl. No.: 15/316,539

(22) PCT Filed: Jun. 17, 2015
(86) PCT No.: PCT/KR2015/006110

§ 371 Date: Dec. 6, 2016

(30) Foreign Application Priority Data
Jun. 17, 2014 (KR) 10-2014-0073670
Jun. 16, 2015 (KR) 10-2015-0084904

(51) Int. Cl. H01L 51/00; C09K 11/06; C07D 333/76; C07C 15/28; C07D 307/91; C07D 307/77; C07D 333/08; C07D 491/048; C09K 11/02; C07D 495/04

(52) U.S. Cl. CPC H01L 51/0074; C09K 11/025; C09K 11/06; H01L 51/0058; H01L 51/006; H01L 51/0072; C07D 333/76; C07D 495/04; H01L 51/0071; H01L 51/0052; C07D 307/91; H01L 51/0073; C07D 307/77; C07D 333/08; H01L 51/0068; C07D 491/048; C07C 15/28; C09K 2211/1007; C09K 2211/1011; C09K 2211/1014; H01L 51/5012

(57) ABSTRACT

The present disclosure relates to an electron buffering material, and an organic electroluminescent device comprising a first electrode, a second electrode facing the first electrode, a light-emitting layer between the first electrode and the second electrode, and an electron transport zone and an electron buffering layer between the light-emitting layer and the second electrode. The organic electroluminescent device comprising the electron buffering material of the present disclosure has a low driving voltage and excellent luminous efficiency.

### TECHNICAL FIELD

The present invention relates to an electron buffering material and an organic electroluminescent device.

### BACKGROUND ART

An organic electroluminescent device (OLED) emitting green light composed of a light-emitting layer and a charge transport layer was developed by Tang et al. of Eastman Kodak in 1987, by using N,N′-diphenyl-N,N′-bis(3-methylphenyl)-(1,1′-biphenyl)-4,4′-diamine (TPD) and tris(8-hydroxyquinolinato)aluminum (Alq3). Afterward, an organic electroluminescent device had been rapidly researched due to the outstanding performance and growth potential in display implementation. Thus, many kinds of an electron transfer material (ETM) have been developed after the hole transport material (HTM). Alq3 has excellent properties in an ETM as well as a light-emitting layer. However, an ETM has a low electron current, which causes blocking the performance improvements of the organic electroluminescent device. Accordingly, the development of materials to replace the conventional ETM such as Alq3 is constantly being demanded.

In particular, a blue device has a low efficiency problem compared to the red and green devices. In order to solve the problems, it is time to require the optimization of the device as well as the development of the materials for the blue device. Recently, there has been research to improve the deterioration characteristic of the device according to the temperature by incorporating an electron buffer layer between the light emitting layer and the electron injection layer and to improve the efficiency by controlling an electron injection.

APPLIED PHYSICS LETTERS 90, 123506, 2007 discloses a blue fluorescent light-emitting device comprising an electron buffering layer. The document recites coordinate shift according to anthracene-based hosts and amine-based dopants focusing on controlling a light-emitting zone by an electron buffering layer and improving color coordinates, and demonstrates the mechanism by Förster energy transfer between the dopants of the electron buffering layer and the light emitting layer, but it only recites the coordinates rather than the improvement of the efficiency.

JP Patent No. 4947909 discloses a blue fluorescent light-emitting device comprising an electron buffering layer, wherein the low driving voltage is achieved by controlling the mobility by efficiently injecting an electron into a light emitting layer against an Alq3 through inserting the electron buffering layer, and the long lifespan is achieved by preventing the degradation of the light emitting interface. However, the document limits the material of an electron buffering layer to Alq3 derivatives, and the object to electronics limitation, and thus the document was a limitation to the analysis of excellent efficiency and various material groups.

### DISCLOSURE OF THE INVENTION

### Problems to be Solved

The objective of the present disclosure is to provide an electron buffering material which can provide an organic electroluminescent device having low driving voltage and excellent luminous efficiency, and an organic electroluminescent device comprising the electron buffering material.

### Solution to Problems

The present inventors found that the objective above can be achieved by an electron buffering material comprising a compound represented by the following formula 1; and an organic electroluminescent device comprising a first electrode, a second electrode facing the first electrode, a light-emitting layer between the first electrode and the second electrode, and an electron transport zone and an electron buffering layer between the light-emitting layer and the second electrode, wherein the electron buffering layer comprises a compound represented by the following formula 1.

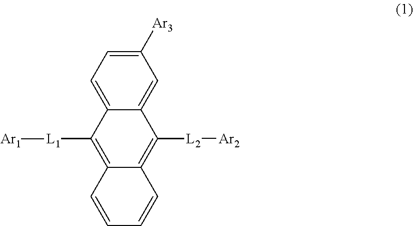

wherein

L₁ and L₂ each independently represent a single bond, a substituted or unsubstituted (C6-C30)arylene, or a substituted or unsubstituted (5- to 40-membered)heteroarylene;

Ar₁ to Ar₃ each independently represent hydrogen, a substituted or unsubstituted (C6-C30)aryl, or a substituted or unsubstituted (5- to 40-membered)heteroaryl;

the heteroaryl(ene) contains at least one hetero atom selected from B, N, O, S, P(═O), Si, and P.

### Effects of the Invention

By using the electron buffering material of the present disclosure, an organic electroluminescent device can secure rapid electric current characteristics by the intermolecular stacking and the interation of interface between an electron buffering layer and a light-emitting layer, thereby being able to have excellent luminous efficiency and low driving voltage.

### BRIEF DESCRIPTION OF THE DRAWINGS

FIG. 1 is a schematic sectional view illustrating a structure of an organic electroluminescent device according to one embodiment of the present disclosure;

FIG. 2 is a schematic sectional view of an energy band diagram among a hole transport layer, a light-emitting layer, an electron buffering layer, and an electron transport zone of an organic electroluminescent device according to one embodiment of the present disclosure; and

FIG. 3 is a graph illustrating a current efficiency versus a luminance of organic electroluminescent devices of Example 1 and Comparative Example 1.

### EMBODIMENTS OF THE INVENTION

Hereinafter, the present disclosure will be described in detail. However, the following description is intended to explain the invention, and is not meant in any way to restrict the scope of the invention.

Herein, “(C1-C30)alkyl(ene)” indicates a linear or branched alkyl(ene) having 1 to 30, preferably 1 to 20, and more preferably 1 to 10 carbon atoms, and includes methyl, ethyl, n-propyl, isopropyl, n-butyl, isobutyl, tert-butyl, etc. “(C3-C30)cycloalkyl” indicates a mono- or polycyclic hydrocarbon having 3 to 30, preferably 3 to 20, and more preferably 3 to 7 carbon atoms and includes cyclopropyl, cyclobutyl, cyclopentyl, cyclohexyl, etc. “(3- to 7-membered) heterocycloalkyl” indicates a cycloalkyl having 3 to 7, preferably 5 to 7 ring backbone atoms including at least one hetero atom selected from B, N, O, S, P(═O), Si, and P, preferably 0, S, and N, and includes tetrahydrofuran, pyrrolidine, thiolan, tetrahydropyran, etc. Furthermore, “(C6-C30)aryl(ene)” indicates a monocyclic or fused ring derived from an aromatic hydrocarbon and having 6 to 30, preferably 6 to 20, and more preferably 6 to 15 ring backbone carbon atoms, includes having a spiro structure, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. “(5- to 40-membered) heteroaryl(ene)” indicates an aryl group having 5 to 40 ring backbone atoms including at least one, preferably 1 to 4, hetero atom selected from the group consisting of B, N, O, S, P(═O), Si, and P; may be a monocyclic ring, or a fused ring condensed with at least one benzene ring; may be partially saturated; may be one formed by linking at least one heteroaryl or aryl group to a heteroaryl group via a single bond(s); includes having a spiro structure; and includes a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, phenoxazinyl, phenanthridinyl, benzodioxolyl, dihydroacridinyl, etc. Furthermore, “halogen” includes F, Cl, Br, and I.

Herein, “substituted” in the expression “substituted or unsubstituted” means that a hydrogen atom in a certain functional group is replaced with another atom or group, i.e. a substituent. The substituents of the substituted alkyl(ene), the substituted alkoxy, the substituted cycloalkyl, the substituted alkylcarbonyl, the substituted alkoxycarbonyl, the substituted arylcarbonyl, the substituted aryl(ene), and the substituted heteroaryl(ene) in L₁ to L₃, L, La, L_{b}, L_{c}, Ar₁ to Ar₃, Ar₂₁ to Ar₂₃, R, R₁₁ to R₂₈, Aa and HAr of the present disclosure, each independently, are at least one selected from the group consisting of deuterium, a halogen, a cyano, a carboxy, a nitro, a hydroxy, a (C1-C30)alkyl, a halo(C1-C30)alkyl, a (C2-C30)alkenyl, a (C2-C30)alkynyl, a (C1-C30)alkoxy, a (C3-C30)cycloalkyl, a (3- to 7-membered)heterocycloalkyl, a (C6-C30)aryloxy, a (3- to 30-membered)heteroaryl unsubstituted or substituted with a (C6-C30)aryl, a (C6-C30)aryl unsubstituted or substituted with a (3- to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

LUMO (“Lowest Unoccupied Molecular Orbital”) and HOMO (“Highest Occupied Molecular Orbital”) have negative energy levels. However, for convenience, LUMO energy level and HOMO energy level are indicated by absolute values in the present disclosure. Thus, the comparison between the LUMO energy level and the HOMO energy level is conducted on the basis of their absolute values.

In the present disclosure, the LUMO energy level and the HOMO energy level are calculated by Density Functional Theory (DFT). The following results according to the relationship of LUMO energy level of the electron buffering layer (Ab) and LUMO energy level of the host (Ah) is to explain the tendency of the device, which may cause the different results depending on the properties of the specific derivative and the stability of the material.

According to one aspect of the present disclosure, an electron buffering material comprising the compound represented by formula 1 is provided. The electron buffering material indicates a material controlling an electron flow. Therefore, the electron buffering material may be, for example, a material trapping electrons, blocking electrons, or lowering an energy barrier between an electron transport zone and a light-emitting layer. Specifically, the electron buffering material may be for an organic electroluminescent device. In the organic electroluminescent device, the electron buffering material may be used for preparing an electron buffering layer, or may be added to another area such as an electron transport zone or a light-emitting layer. The electron buffering layer may be formed between a light-emitting layer and an electron transport zone, or between an electron transport zone and a second electrode of an organic electroluminescent device. The electron buffering material may be a mixture or composition which may further comprise a conventional material for preparing an organic electroluminescent device.

In formula 1, L₁ and L₂, each independently, may represent preferably, a single bond or a substituted or unsubstituted (C6-C30)arylene; specifically, a single bond, a substituted or unsubstituted phenylene, a substituted or unsubstituted naphthylene, a substituted or unsubstituted anthracenylene, a substituted or unsubstituted phenanthrenylene, a substituted or unsubstituted fluorenylene, a substituted or unsubstituted phenylnaphthylene, or a substituted or unsubstituted naphthylphenylene.

In formula 1, Ar₁ and Ar₂, each independently, may represent preferably, a substituted or unsubstituted (C6-C30)aryl, or a substituted or unsubstituted (5- to 35-membered)heteroaryl. Specifically, Ar₁ and Ar₂ each independently are selected from the group consisting of the following formulae 2-1 to 2-8.

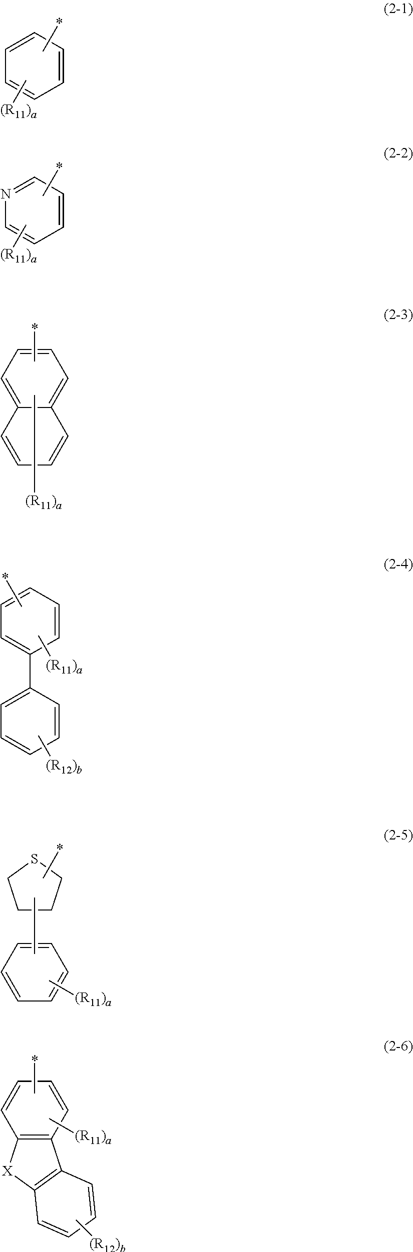

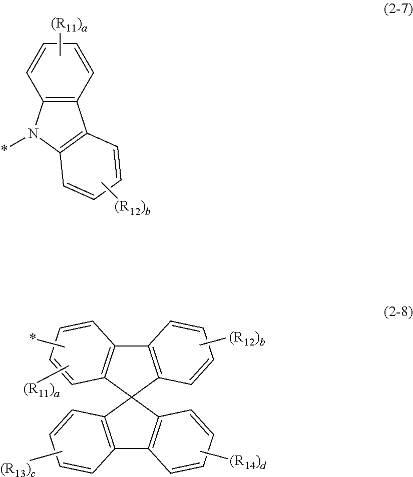

wherein

R₁₁ to R₁₄, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered) heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a substituted or unsubstituted, mono- or polycyclic, (C3-C30) alicyclic or aromatic ring, whose carbon atom(s) may be replaced with at least one hetero atom selected from nitrogen, oxygen, and sulfur;

X represents —S—, —O—, —NR₁₅—, or —CR₁₆R₁₇—;

R₁₅ to R₁₇, each independently, represent hydrogen, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or a substituted or unsubstituted (3- to 7-membered) heterocycloalkyl;

the heteroaryl(ene) and the heterocycloalkyl, each independently, contain at least one hetero atom selected from B, N, O, S, P(═O), Si, and P;

a represents an integer of 1 to 7; b represents an integer of 1 to 5; c and d, each independently, represent an integer of 1 to 4; where a to d are an integer of 2 or more, each of R₁₁ to R₁₄ may be the same or different.

Specifically, R₁₅ to R₁₇, each independently, may represent hydrogen, a substituted or unsubstituted (C1-C10)alkyl, or a substituted or unsubstituted (C5-C18)aryl.

Specifically, R₁₁ to R₁₄, each independently, may represent hydrogen, a substituted or unsubstituted (C6-C18)aryl, or a substituted or unsubstituted (5- to 18-membered)heteroaryl, or may be linked to an adjacent substituent(s) to form a substituted or unsubstituted, mono- or polycyclic, (C5-C18) aromatic ring, whose carbon atom(s) may be replaced with one to three hetero atoms selected from nitrogen, oxygen, and sulfur; and more specifically, each independently, may represent hydrogen, a substituted or unsubstituted phenyl, a substituted or unsubstituted carbazolyl, a substituted or unsubstituted dibenzofuranyl, a substituted or unsubstituted dibenzothiophenyl, or a substituted or unsubstituted fluorenyl, or may be linked to an adjacent substituent(s) to form a phenyl ring, or any one of the following formulae 3-1 and 3-2.

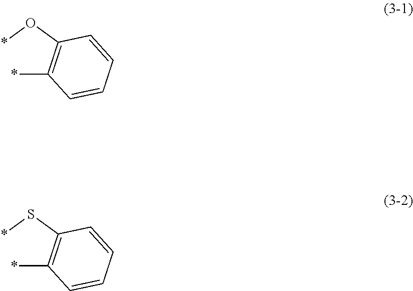 [SMILES: *Oc1ccccc1* ; *Sc1ccccc1*]

In formula 1, Ar₃ may represent preferably hydrogen, or a substituted or unsubstituted (C6-C30)aryl. Specifically, Ar₃ may be represented by the following formula 4.

*-(La)ᵣ-Aa (4)

In formula 4, La represents a single bond, a substituted or unsubstituted (C1-C20)alkylene, or a substituted or unsubstituted (C6-C30)arylene; Aa represents a substituted or unsubstituted (C1-C20)alkyl, or a substituted or unsubstituted (C6-C30)aryl; r represents an integer of 1 or 2; and * represents a bonding site.

Specifically, in formula 4, La may represent a single bond, a substituted or unsubstituted phenylene, a substituted or unsubstituted biphenylene, a substituted or unsubstituted naphthylene, a substituted or unsubstituted anthracenylene, a substituted or unsubstituted phenanthrenylene, or a substituted or unsubstituted fluoreonylene, where r is 2, each of La may be the same or different. Also specifically, in formula 4, Aa may represent a substituted or unsubstituted (C1-C4)alkyl, a substituted or unsubstituted phenyl, a substituted or unsubstituted biphenyl, a substituted or unsubstituted naphthyl, a substituted or unsubstituted anthracenyl, a substituted or unsubstituted phenanthrenyl, or a substituted or unsubstituted fluoreonyl. The substituents of the substituted alkyl(ene), the substituted aryl(ene), etc., in La and Aa, each independently, may represent specifically a (C1-C10)alkyl or deuterium.

Specifically, the compound of formula 1 includes the following, but is not limited thereto.

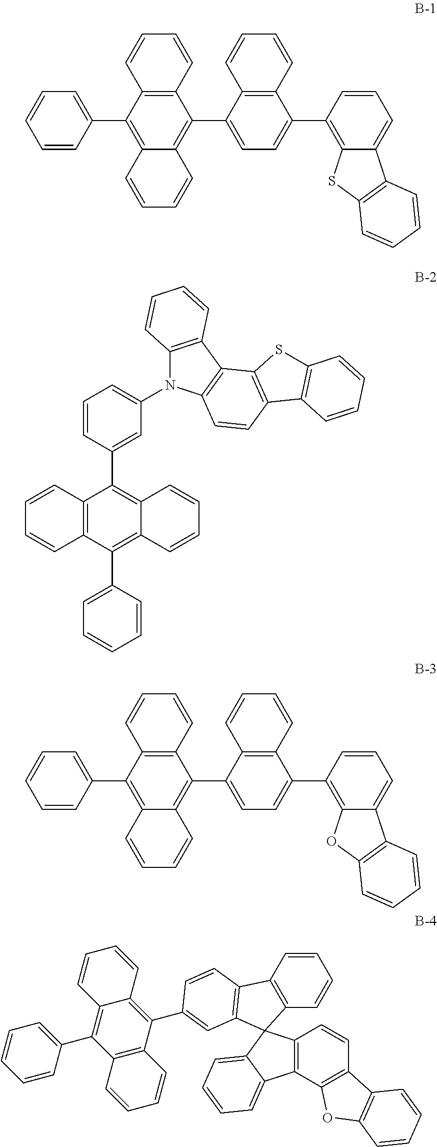 [SMILES: c1ccc(-c2c3ccccc3c(-c3ccc(-c4cccc5c4sc4ccccc45)c4ccccc34)c3ccccc23)cc1 ; c1ccc(-c2c3ccccc3c(-c3cccc(-n4c5ccccc5c5c6sc7ccccc7c6ccc54)c3)c3ccccc23)cc1 ; c1ccc(-c2c3ccccc3c(-c3ccc(-c4cccc5c4oc4ccccc45)c4ccccc34)c3ccccc23)cc1 ; c1ccc(-c2c3ccccc3c(-c3ccc4c(c3)C3(c5ccccc5-4)c4ccccc4-c4c3ccc3c4oc4ccccc43)c3ccccc23)cc1]

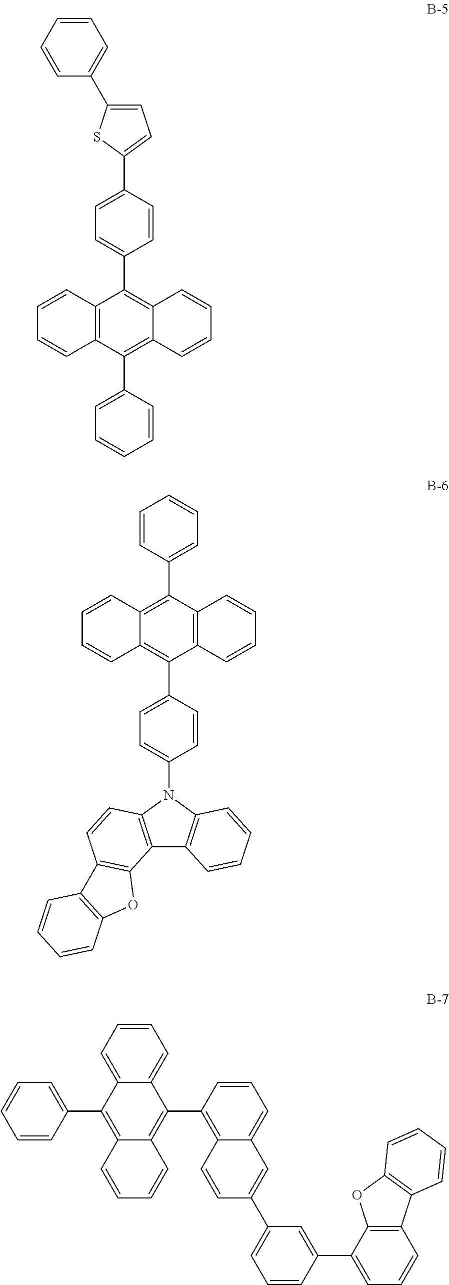 [SMILES: c1ccc(-c2ccc(-c3ccc(-c4c5ccccc5c(-c5ccccc5)c5ccccc45)cc3)s2)cc1 ; c1ccc(-c2c3ccccc3c(-c3ccc(-n4c5ccccc5c5c6oc7ccccc7c6ccc54)cc3)c3ccccc23)cc1 ; c1ccc(-c2c3ccccc3c(-c3cccc4cc(-c5cccc(-c6cccc7c6oc6ccccc67)c5)ccc34)c3ccccc23)cc1]

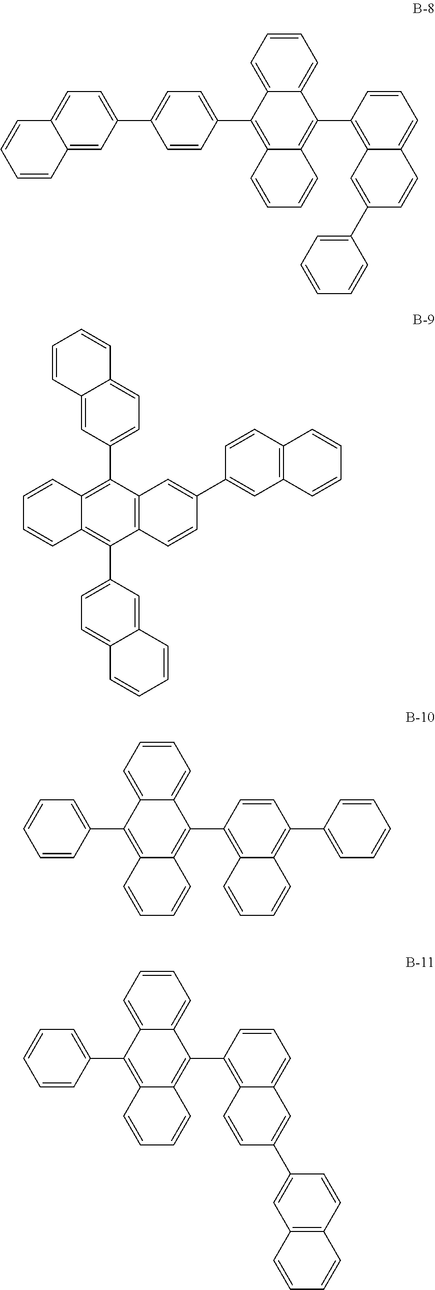

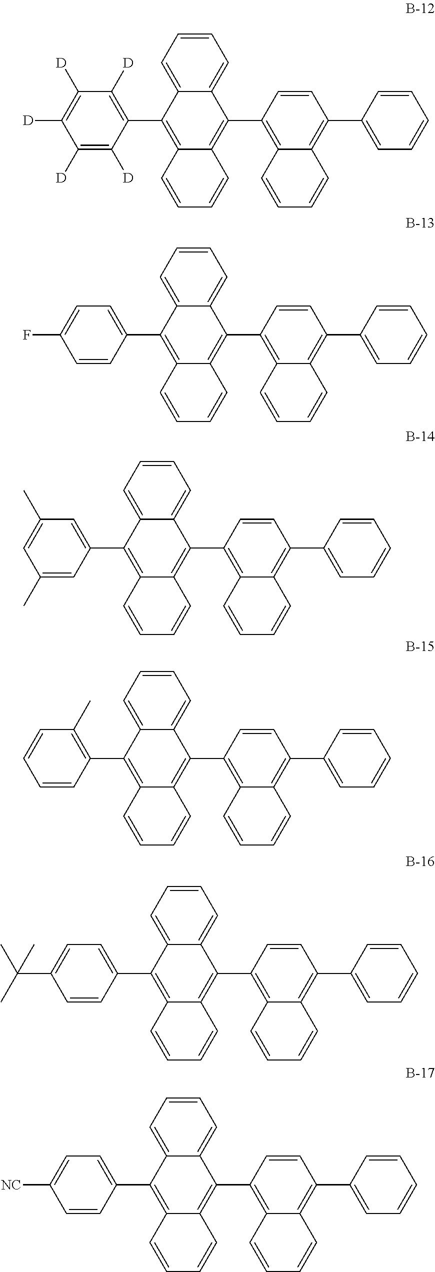

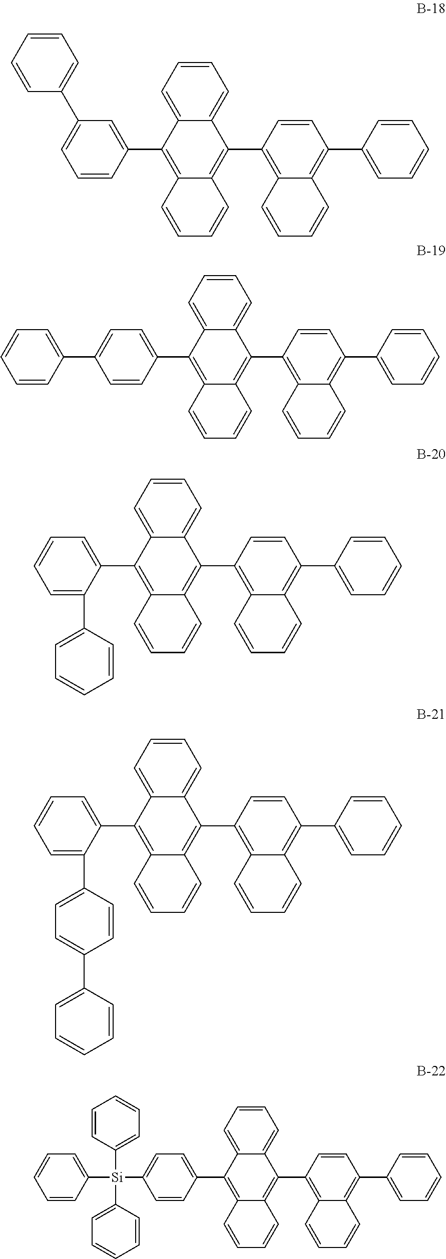

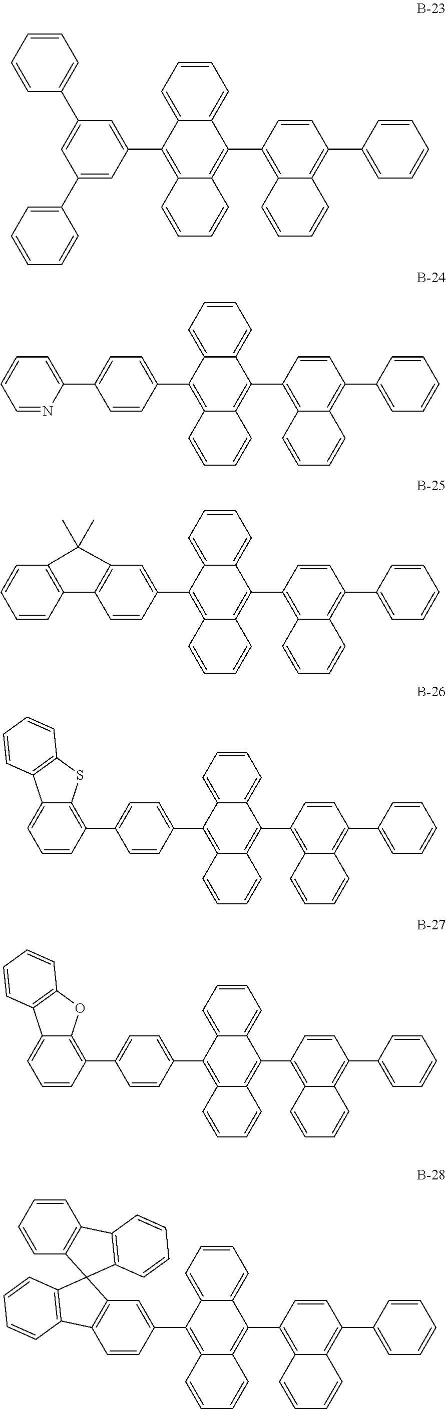

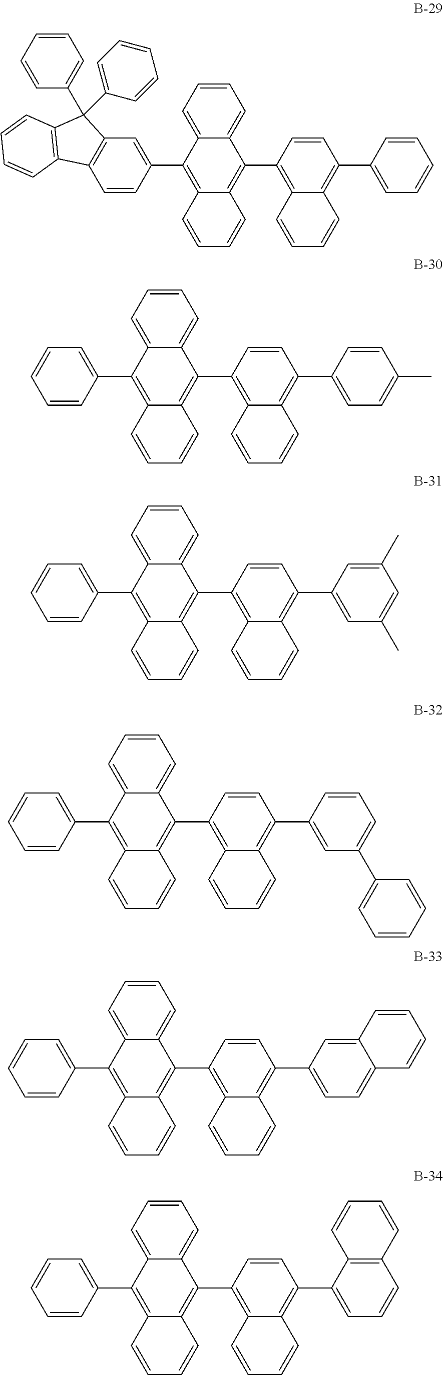

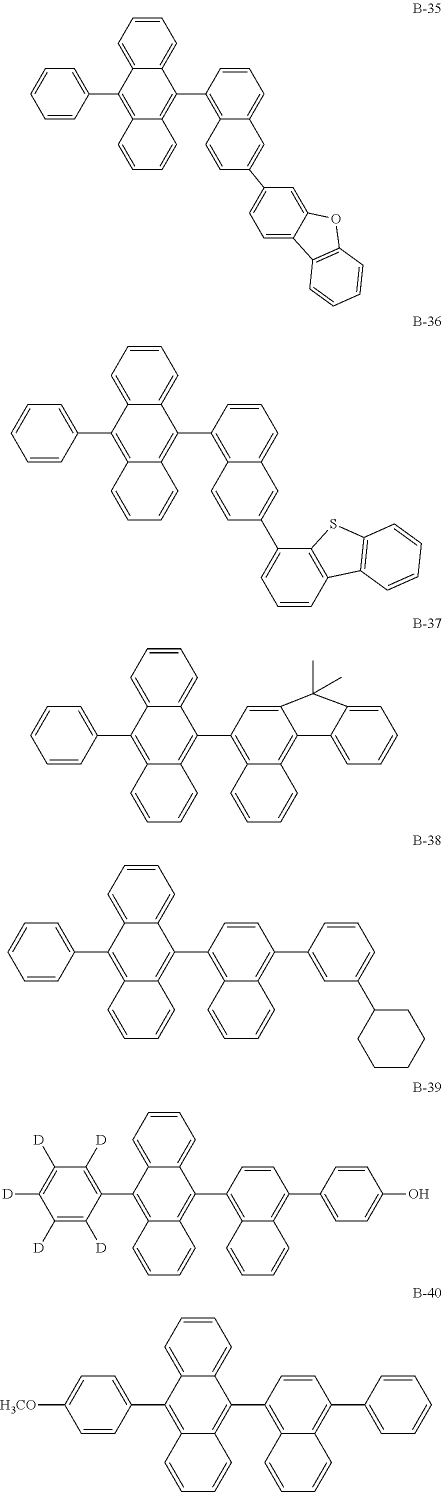

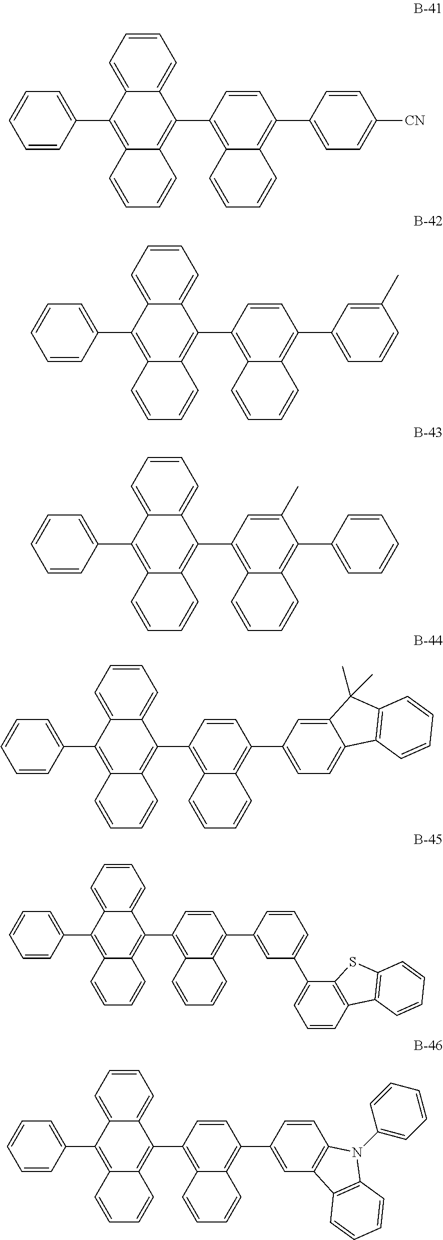

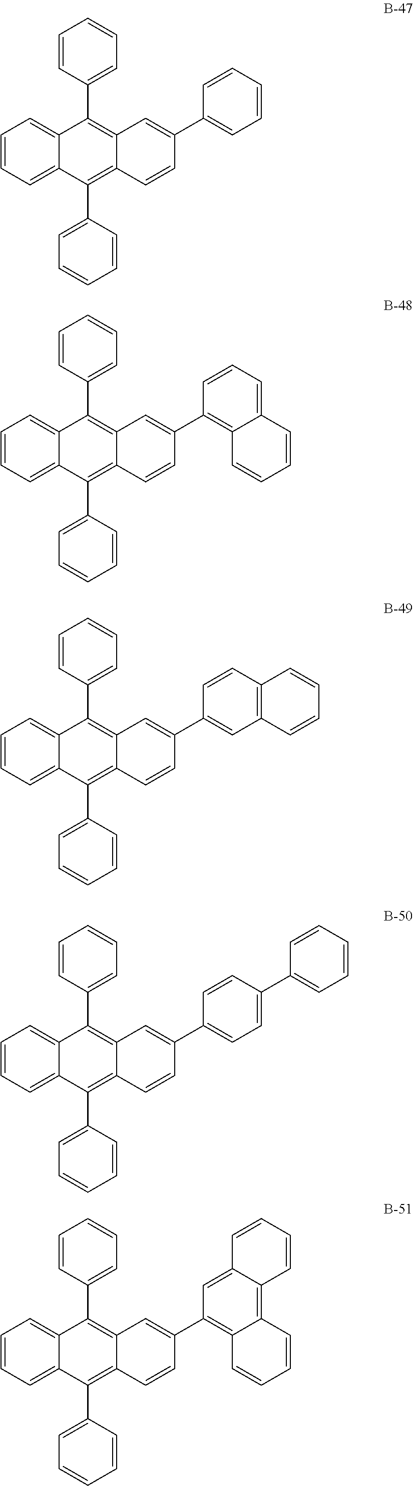

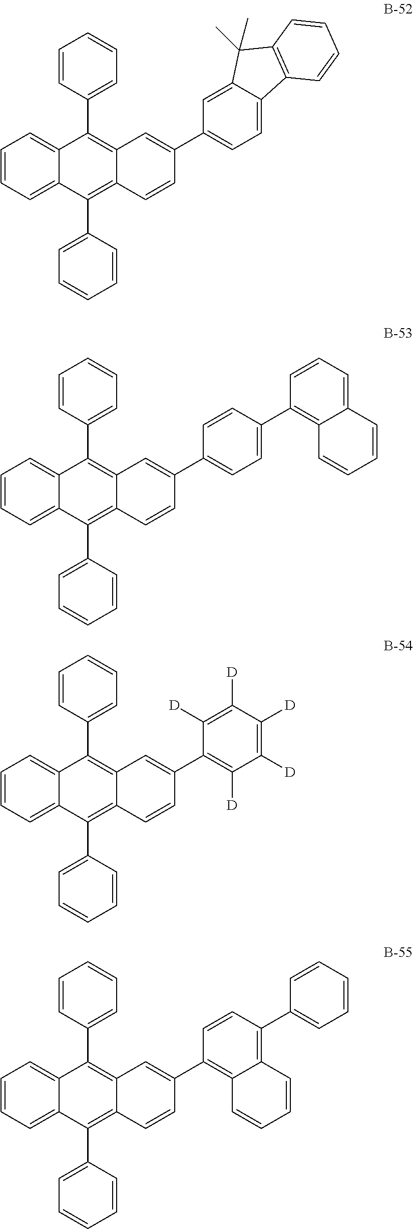

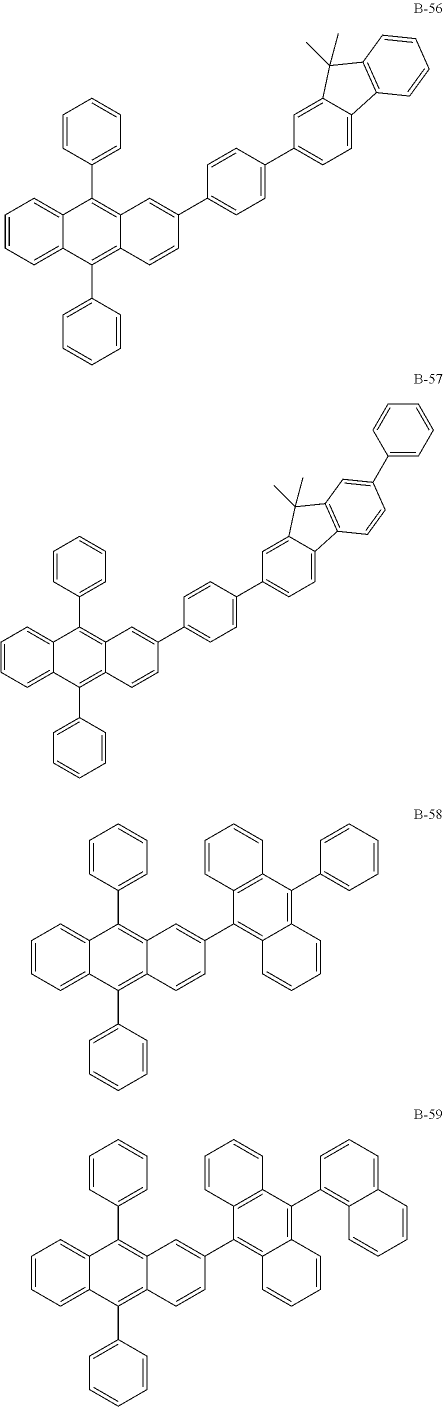

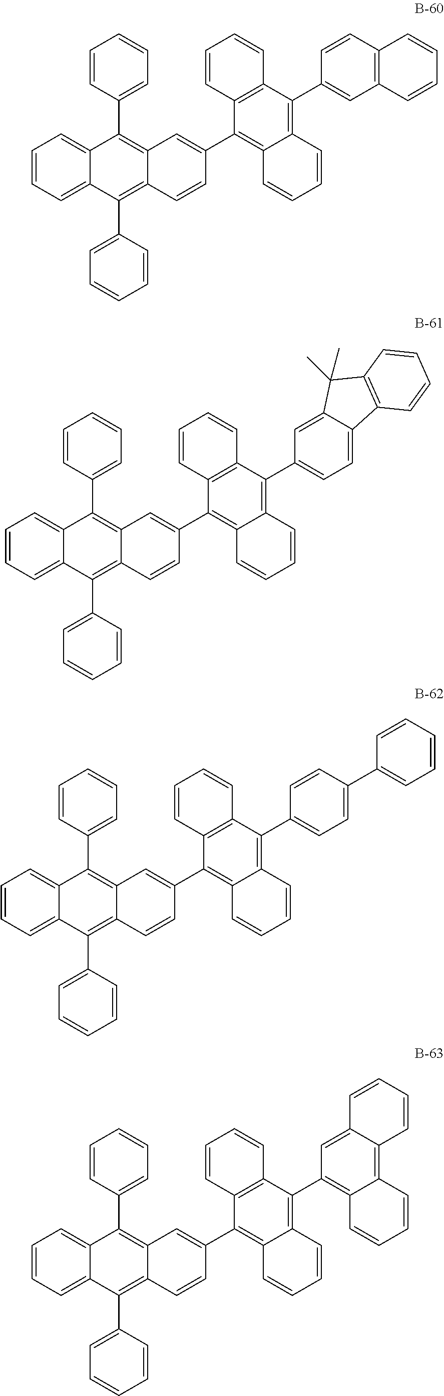

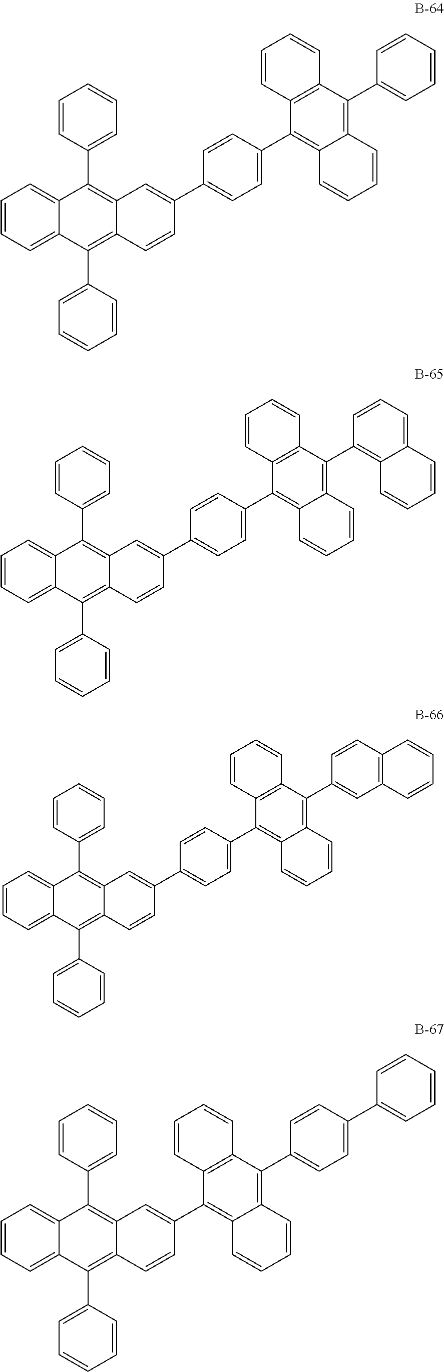

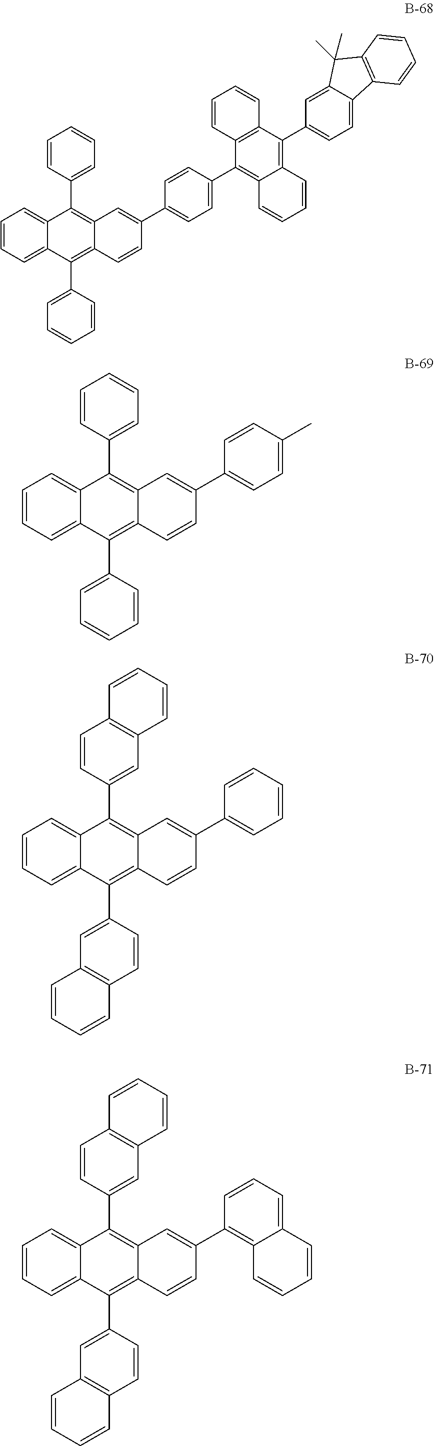

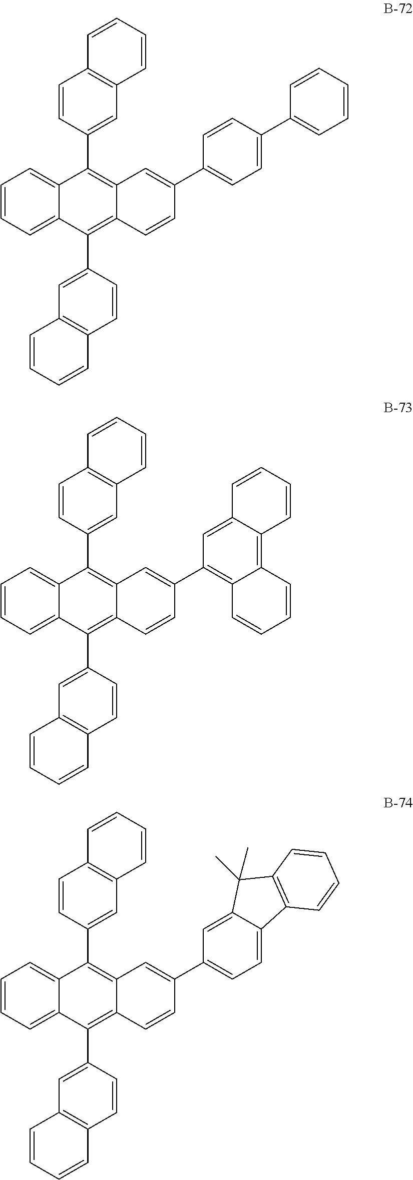

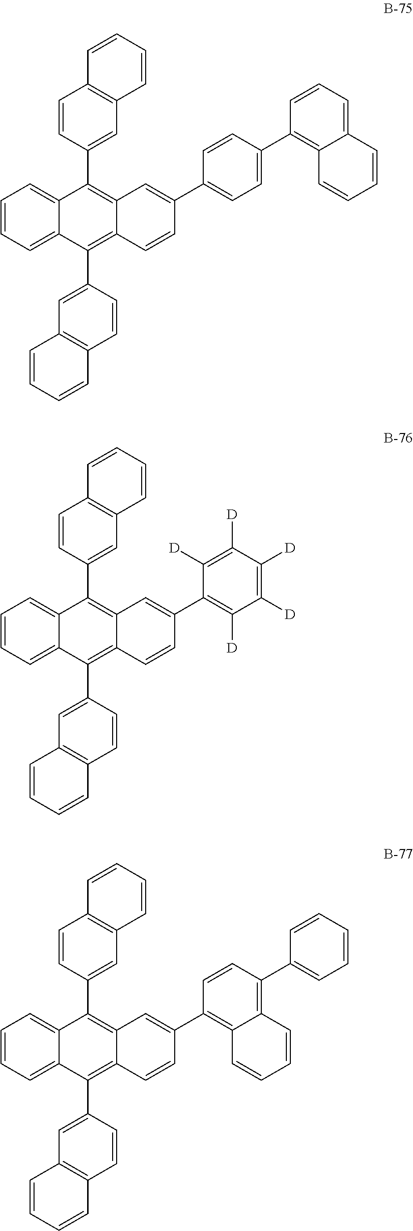

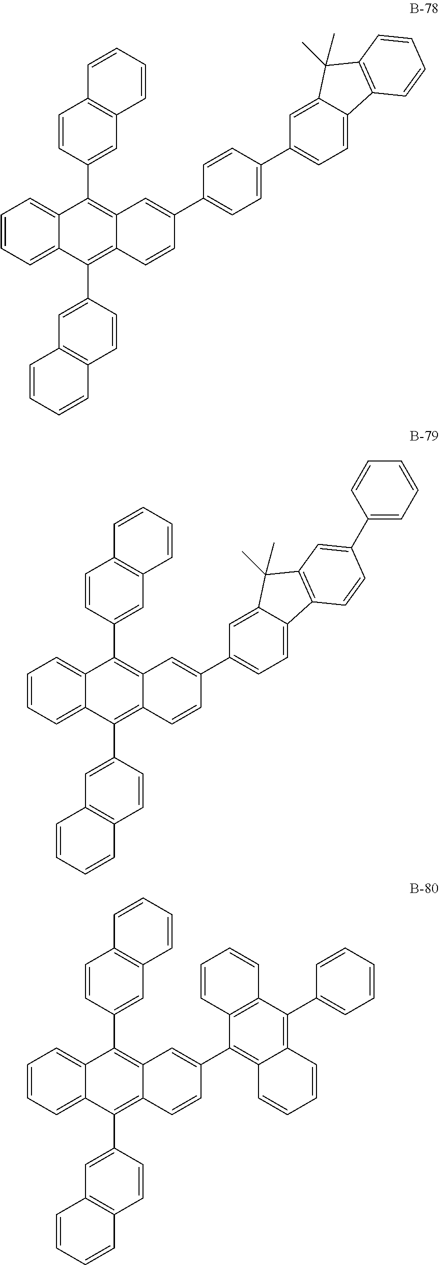

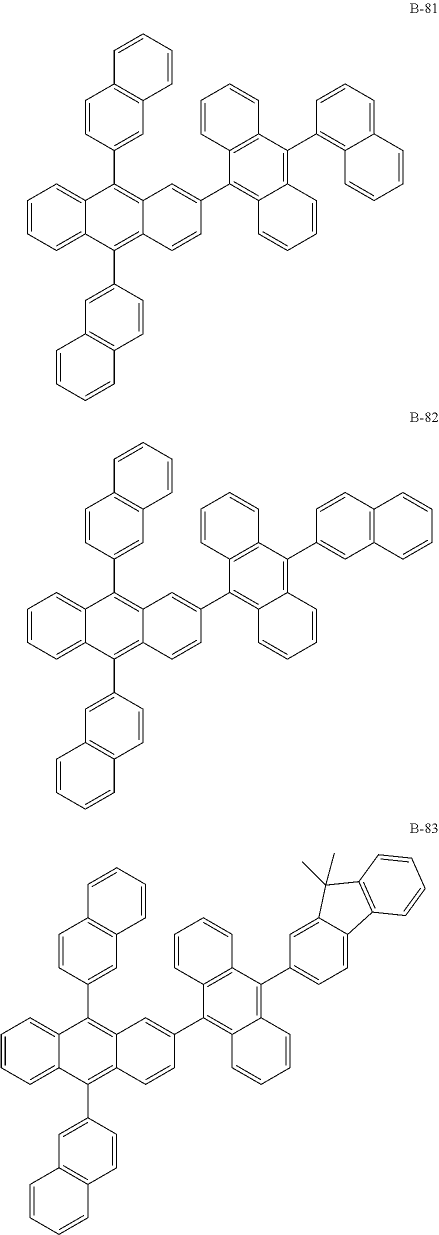

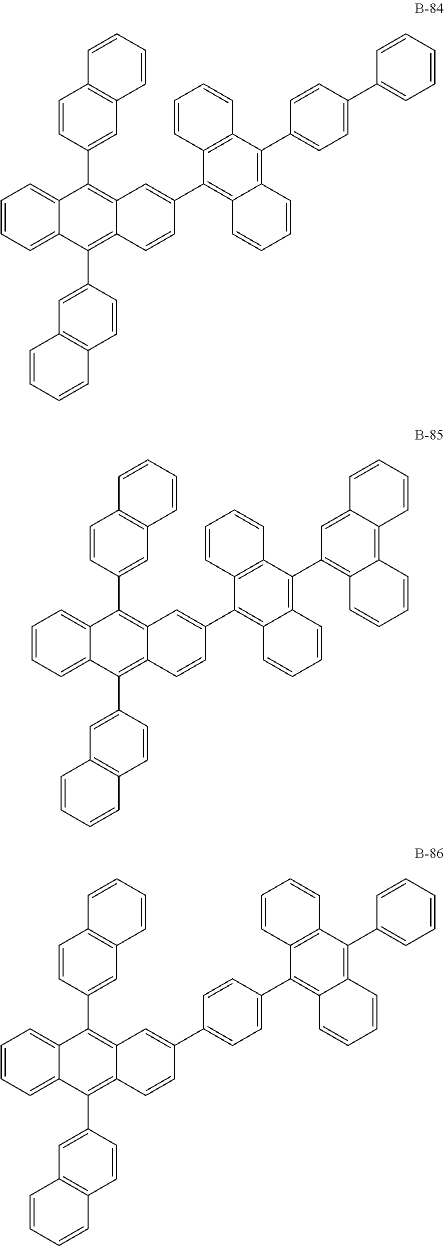

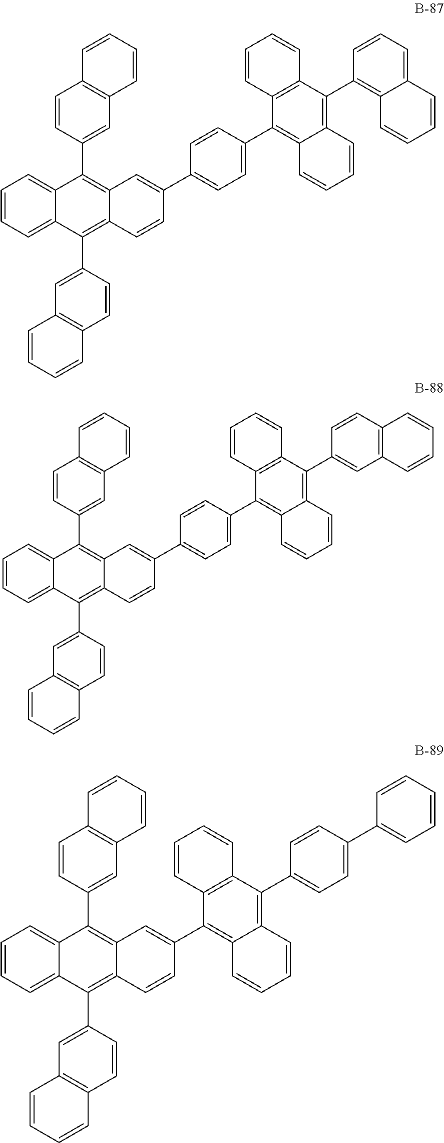

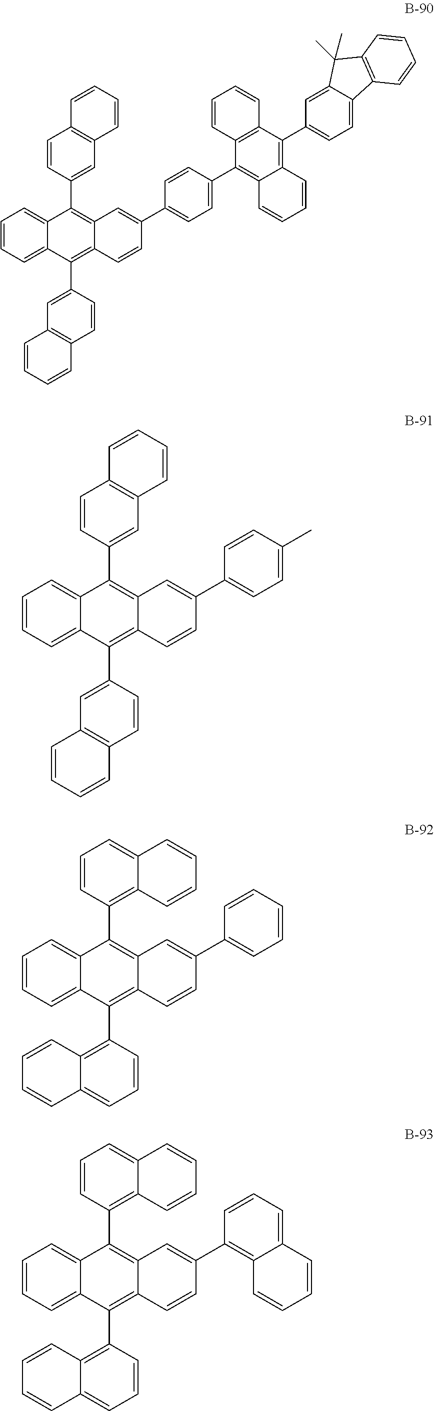

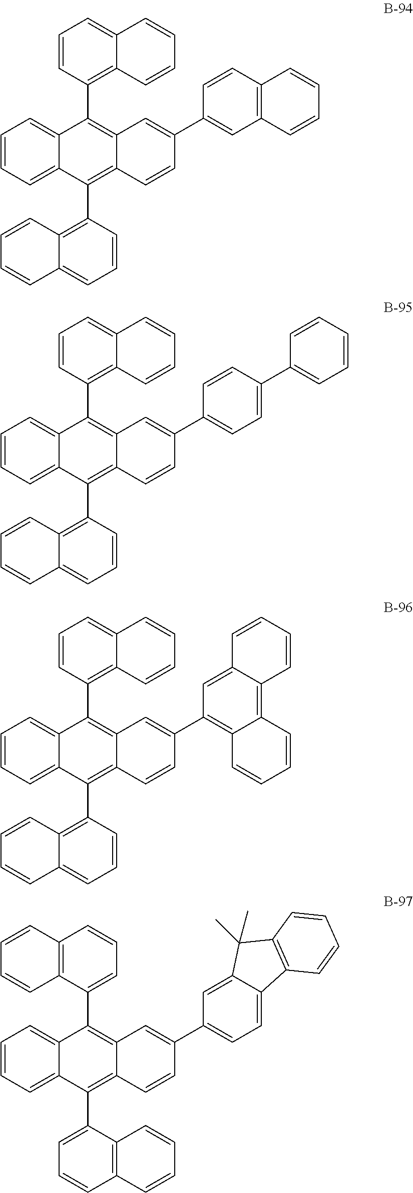

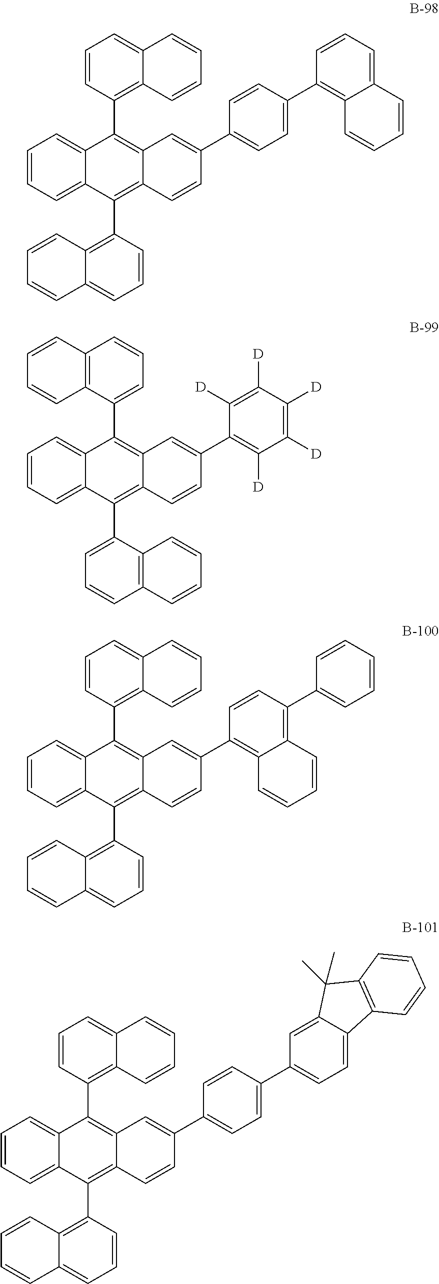

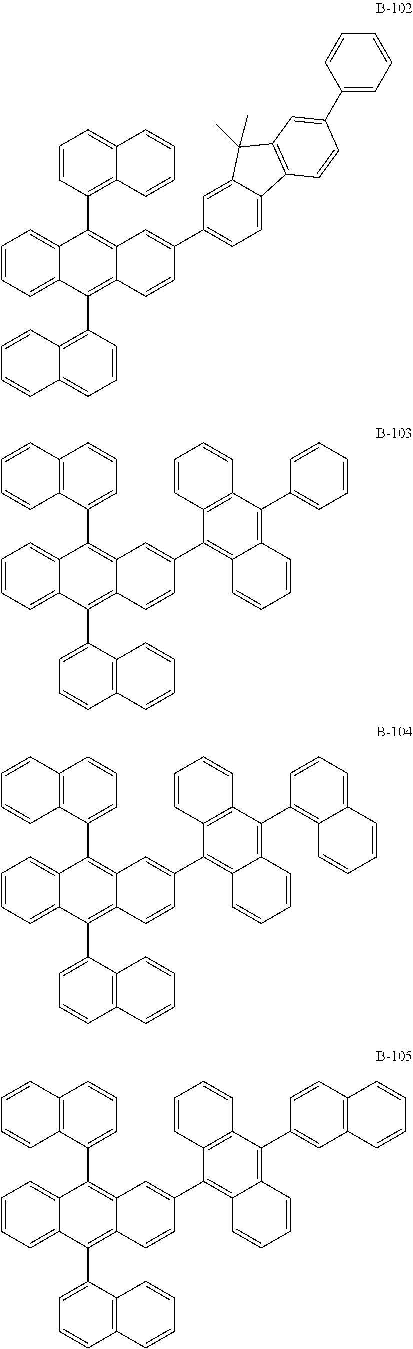

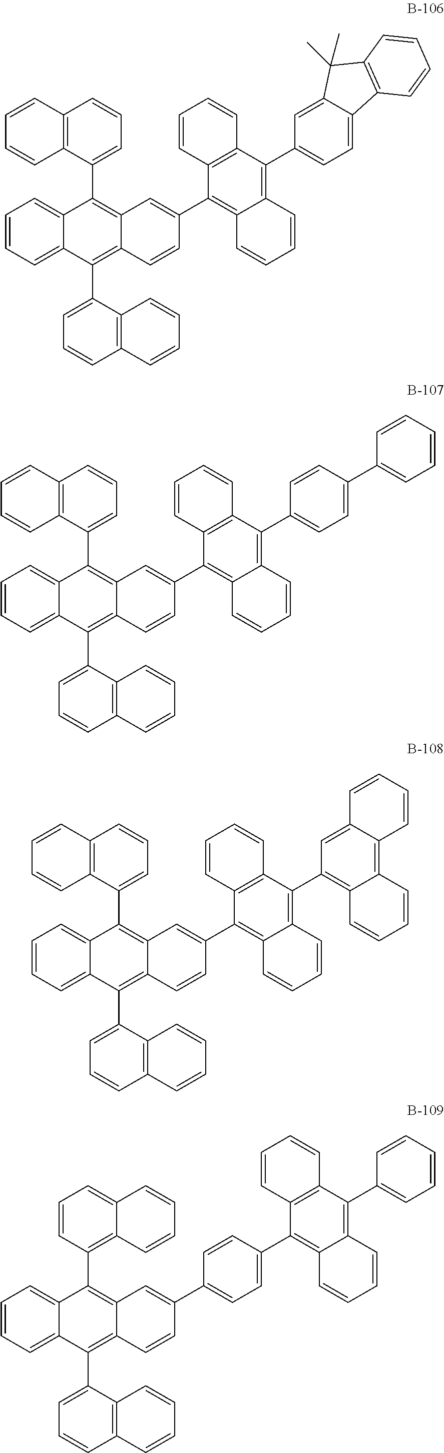

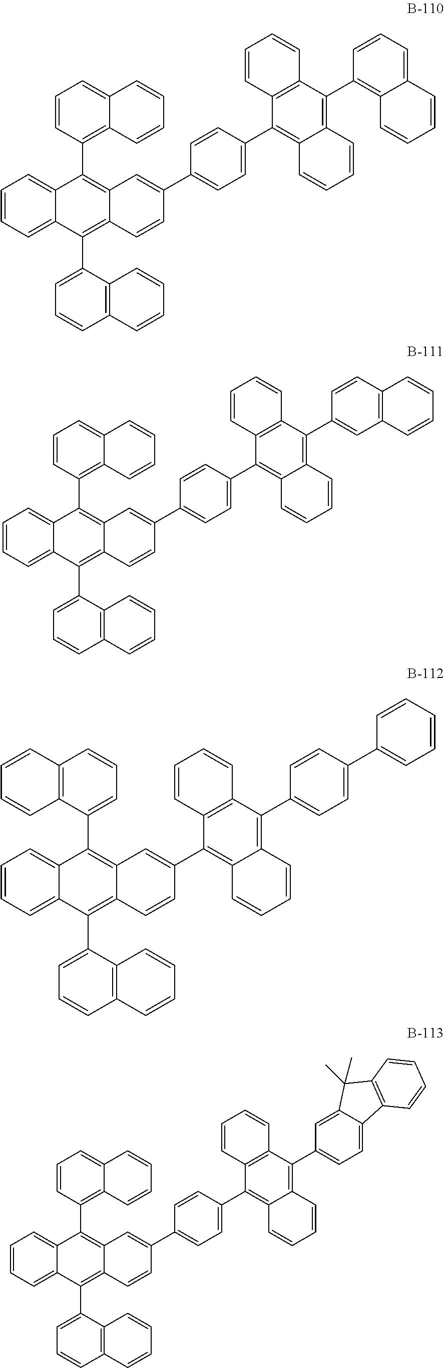

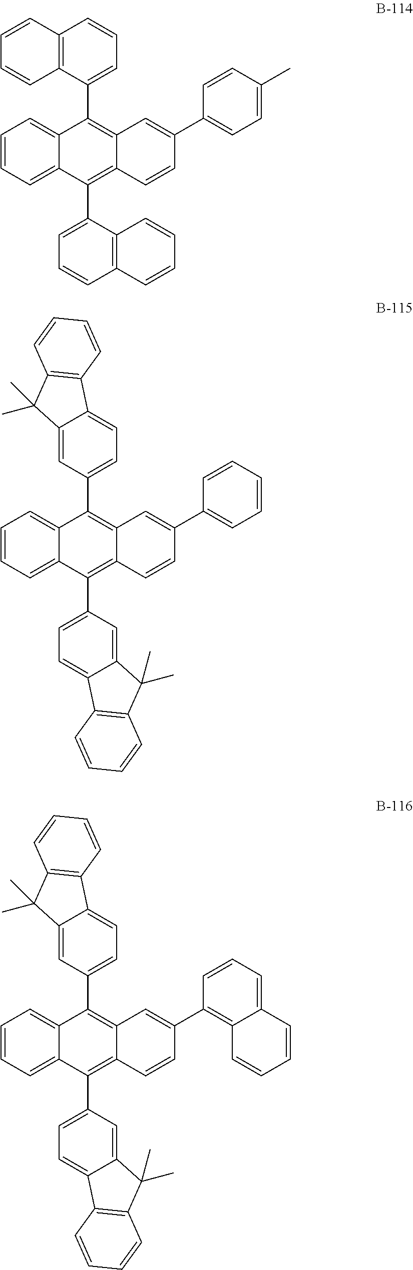

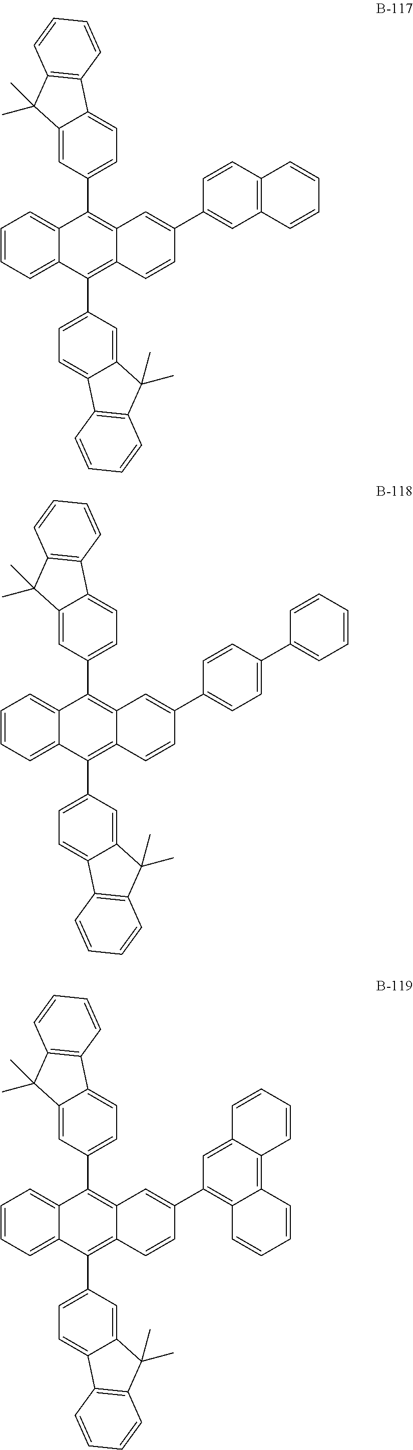

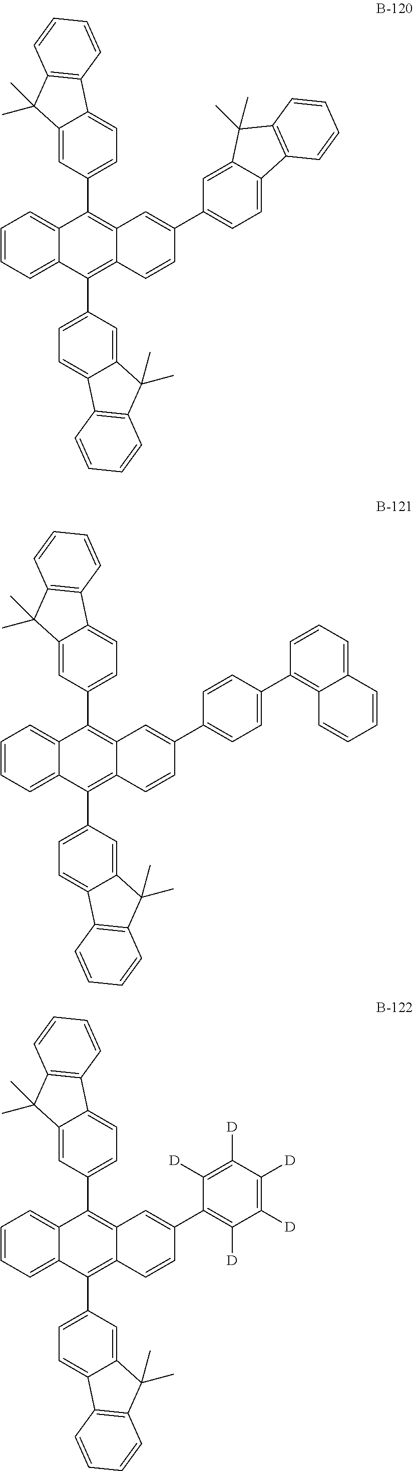

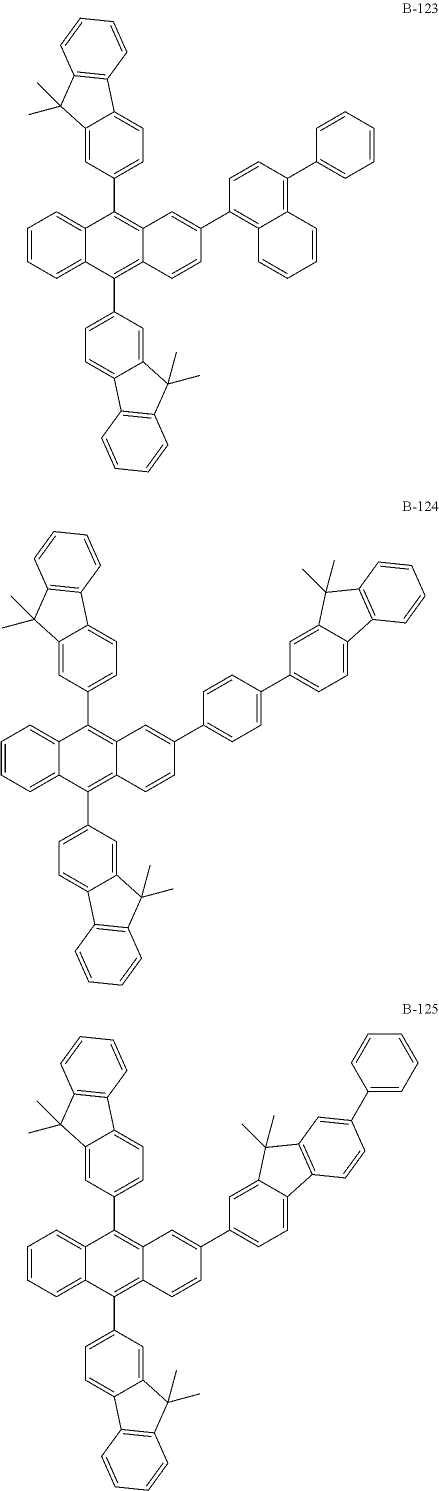

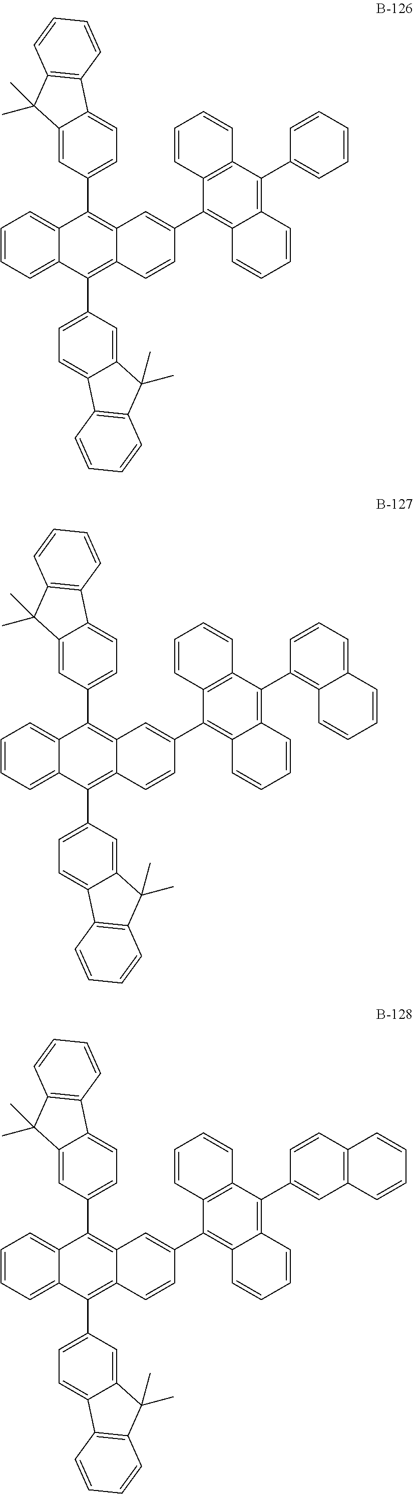

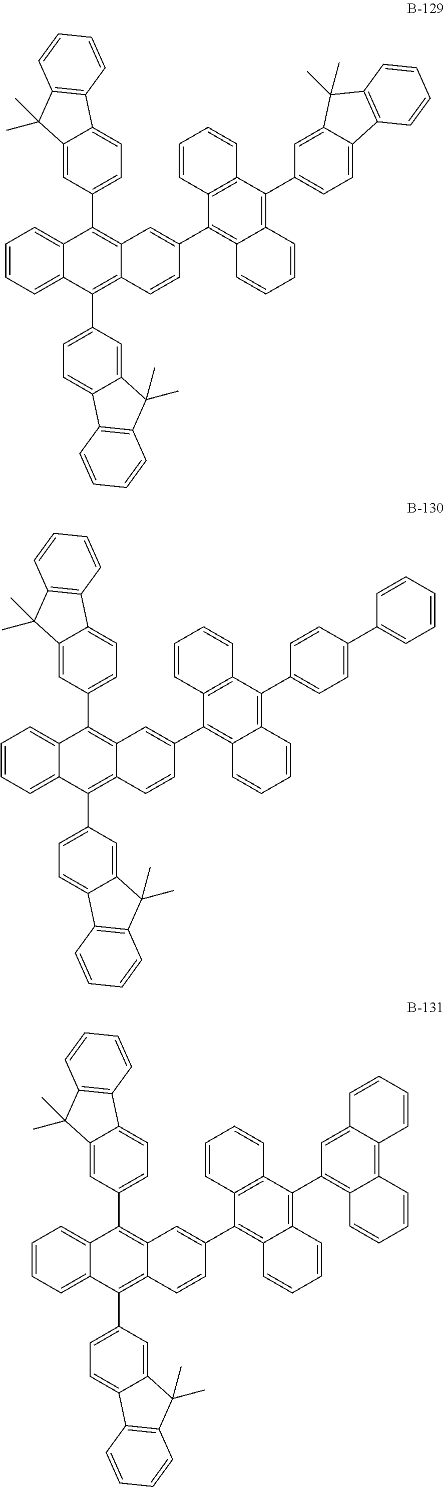

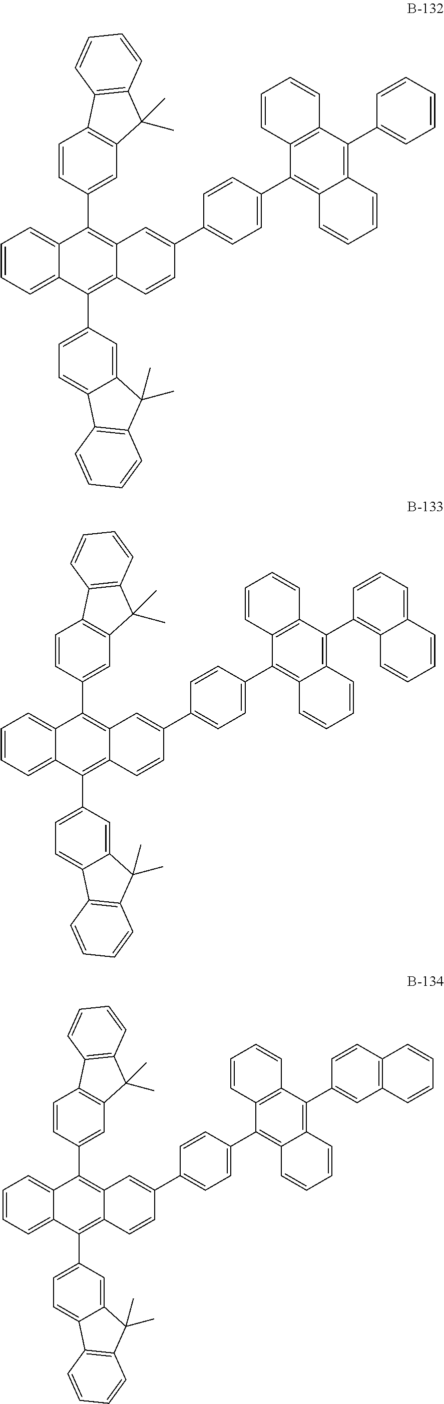

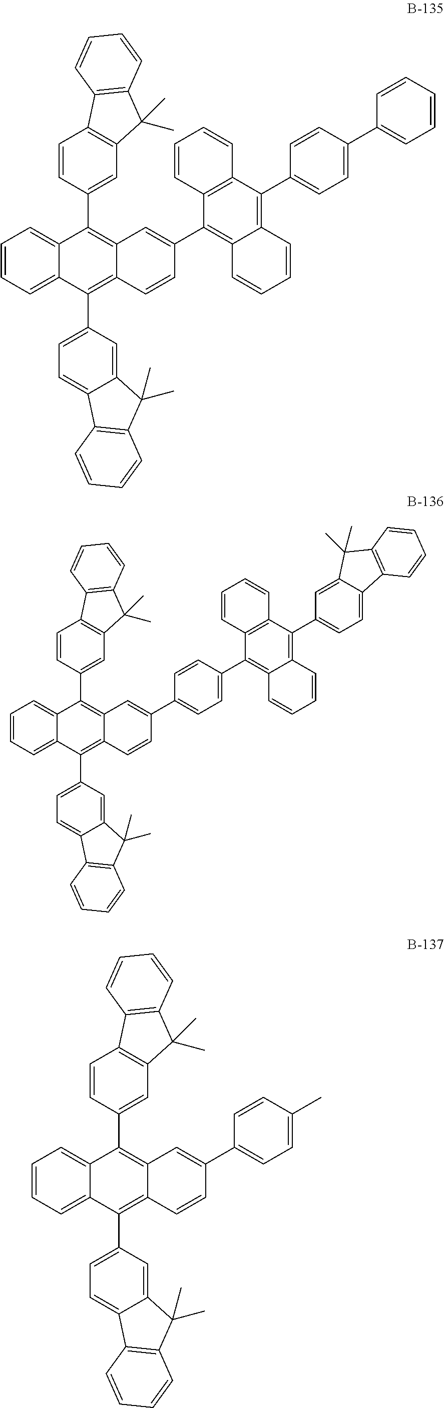

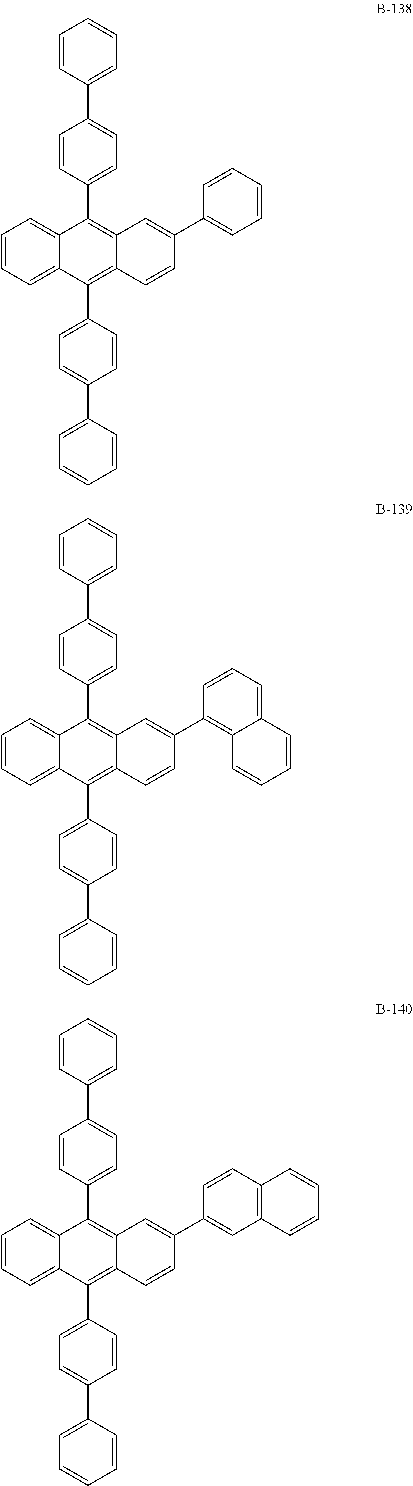

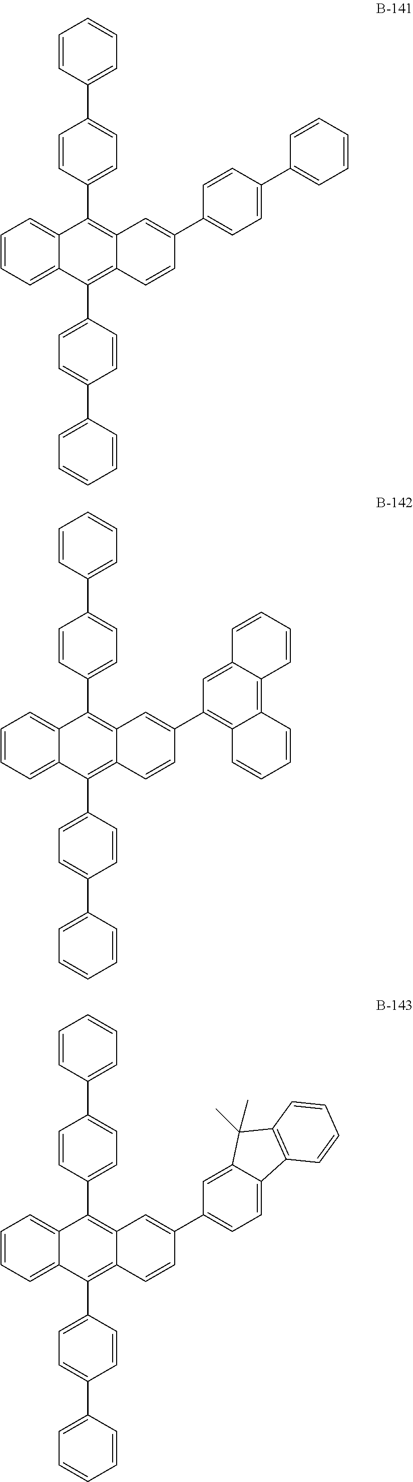

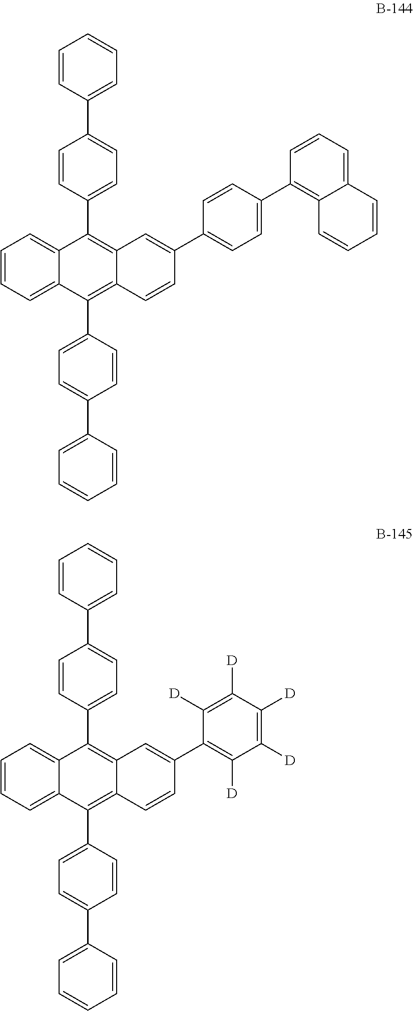

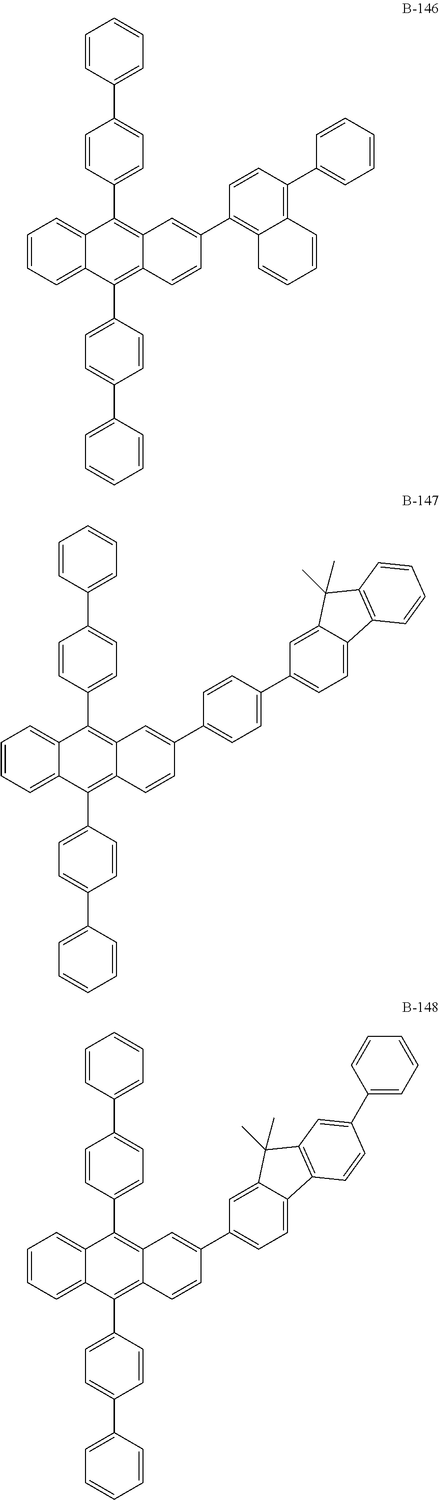

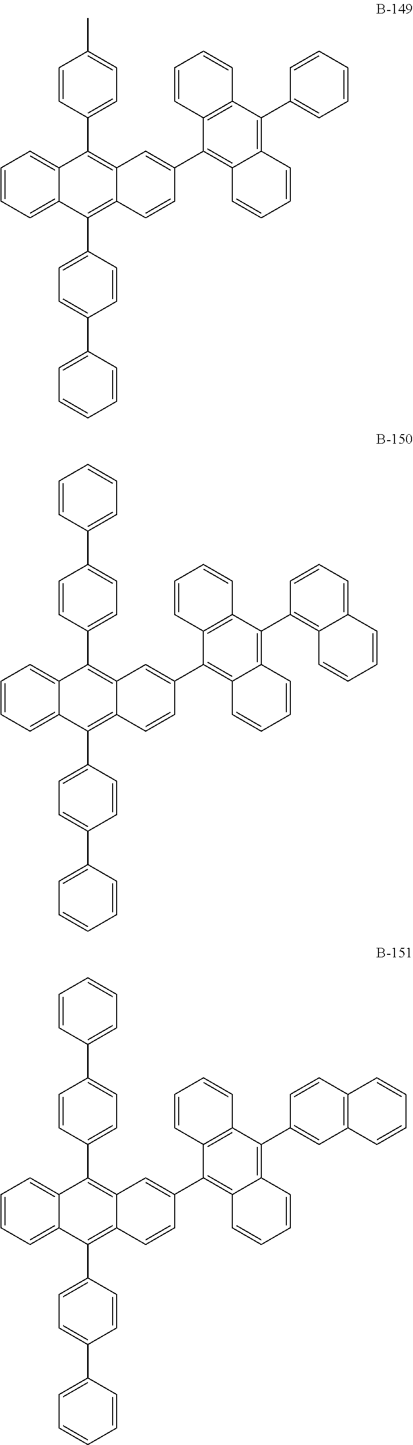

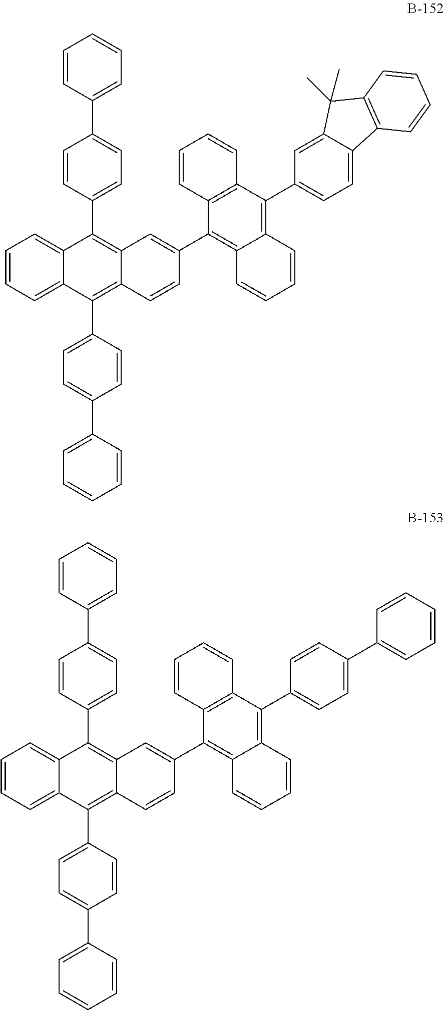

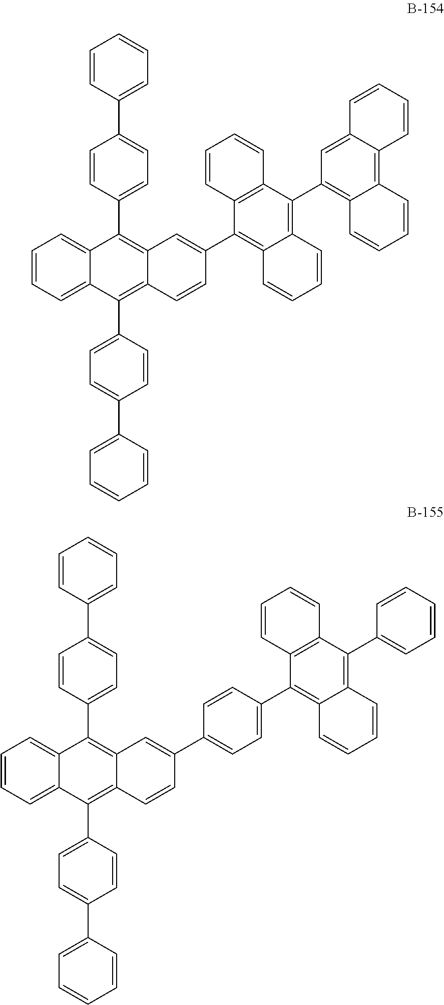

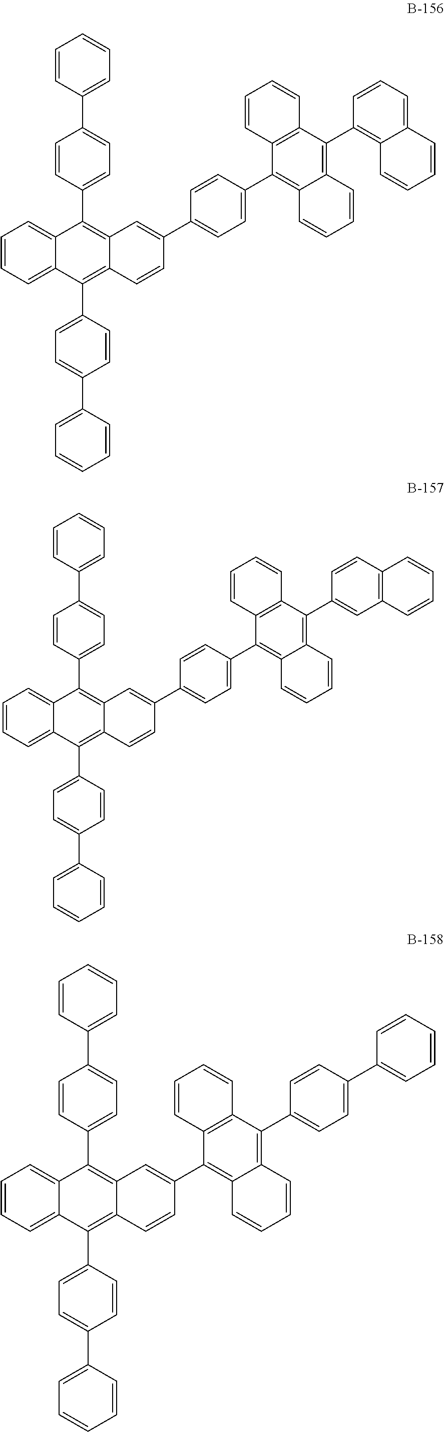

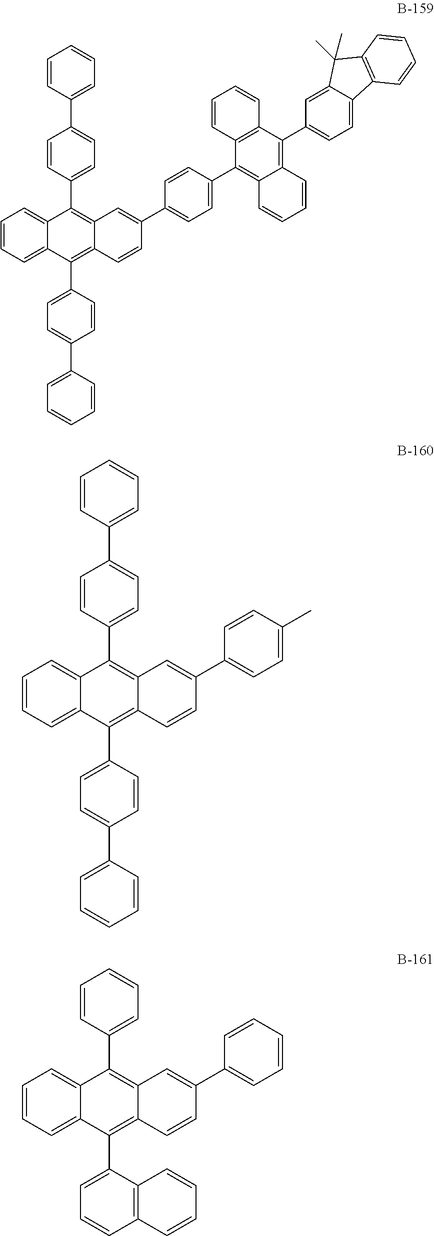

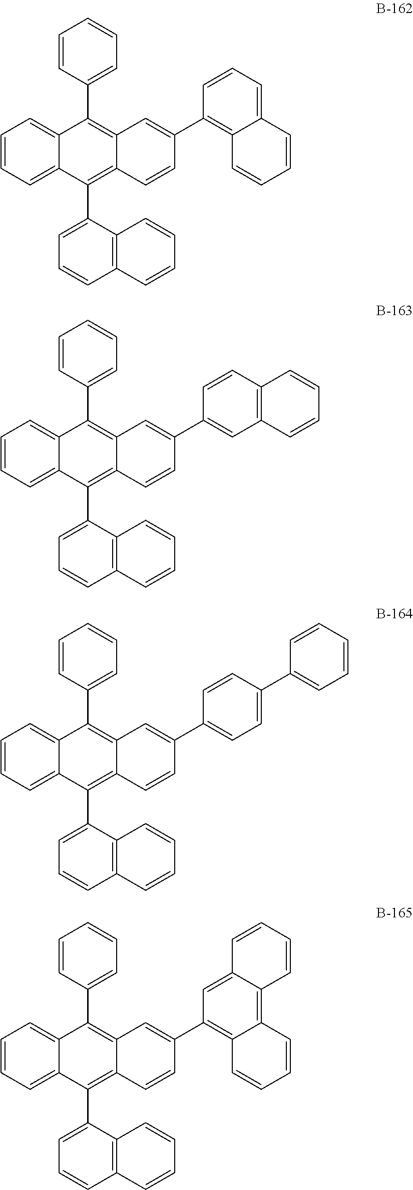

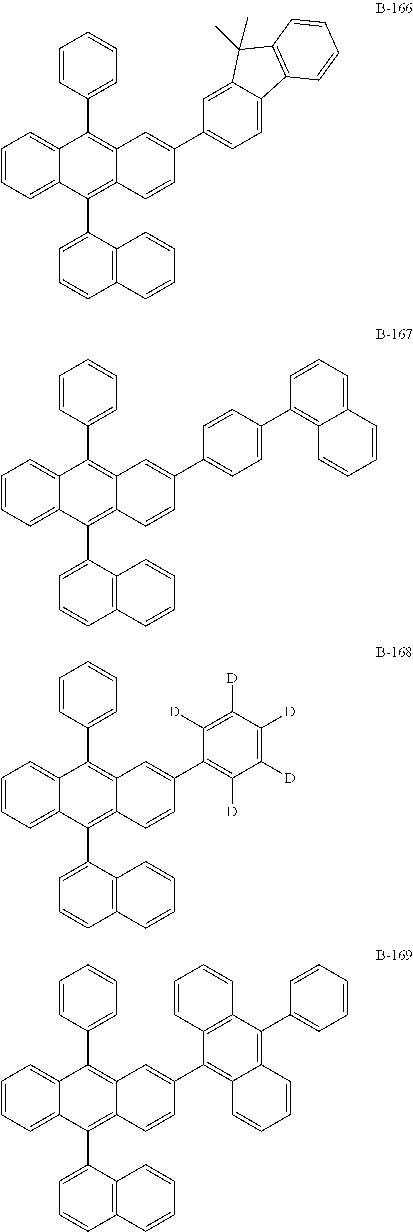

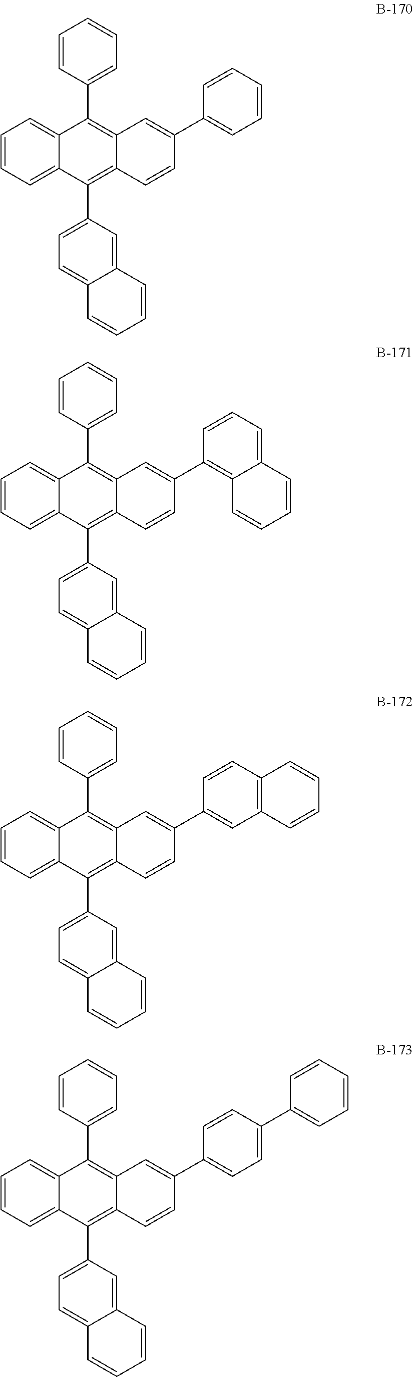

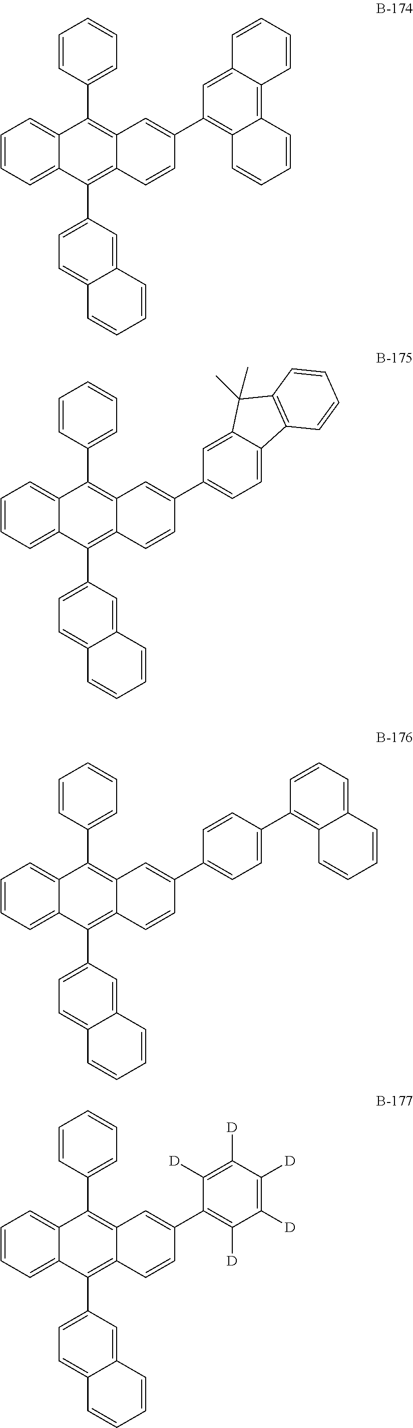

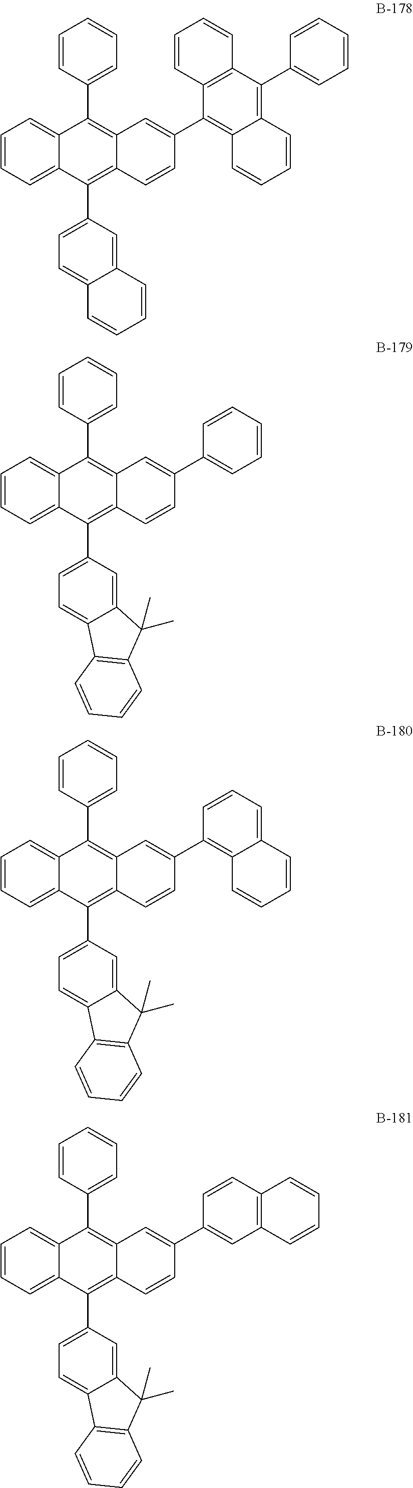

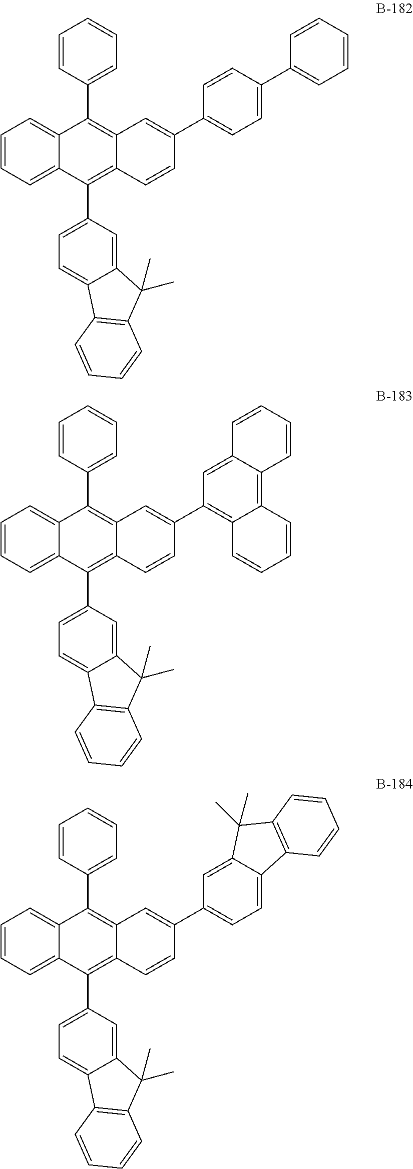

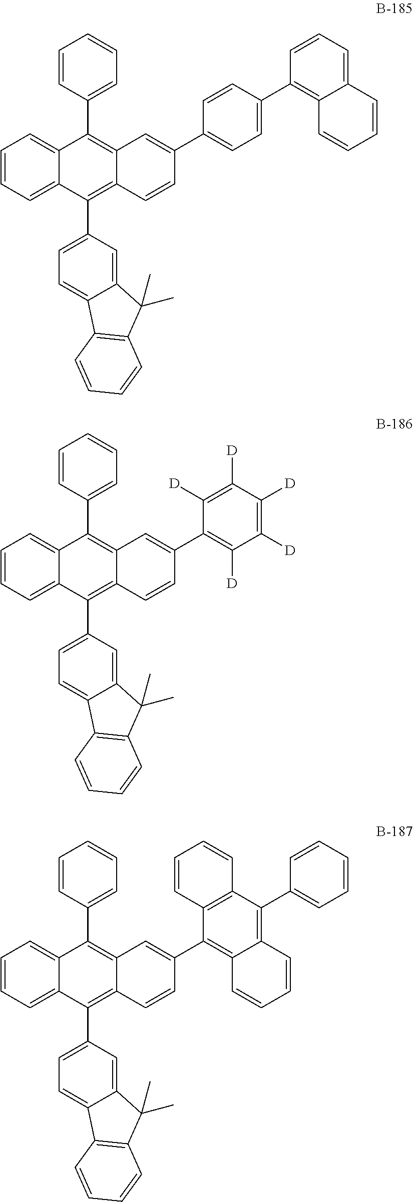

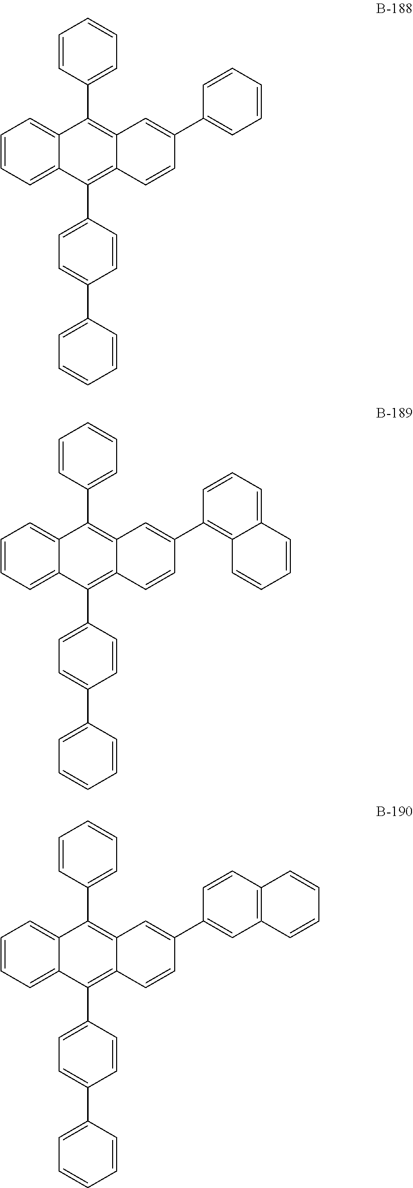

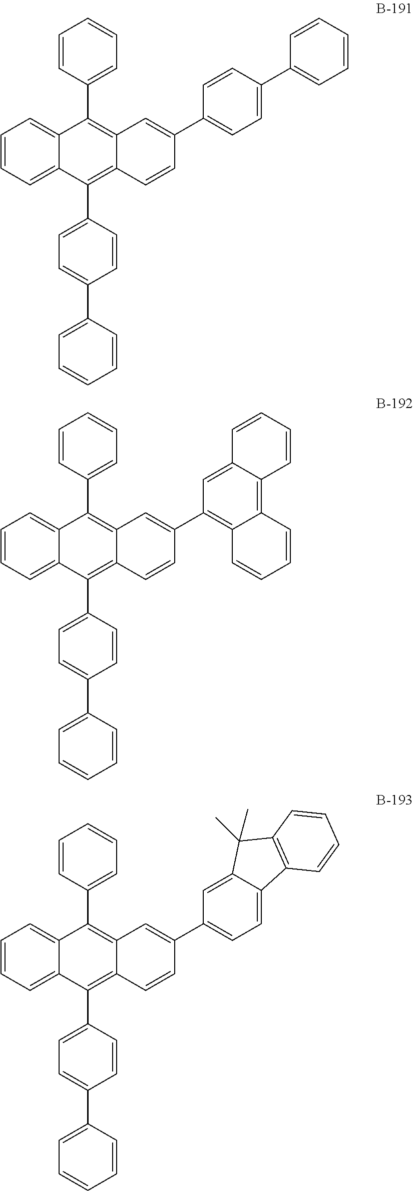

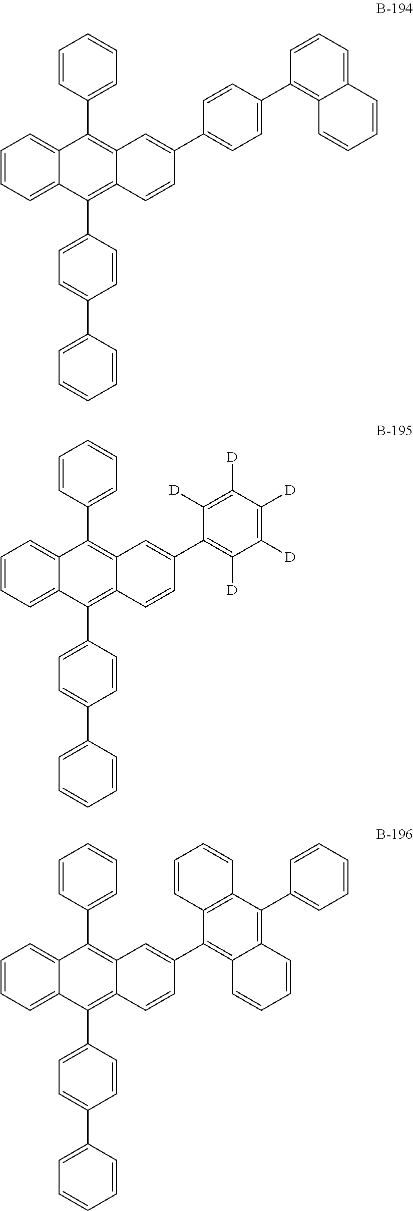

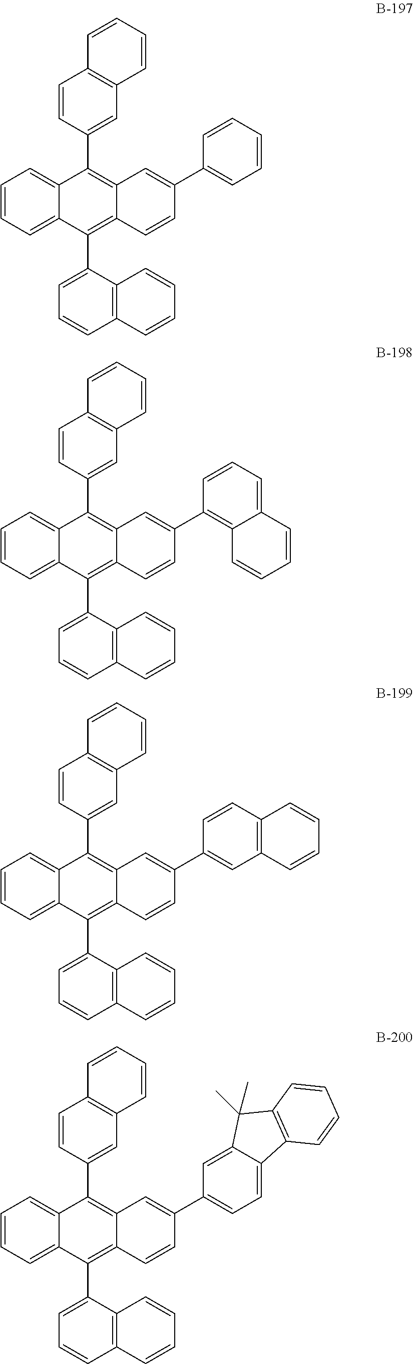

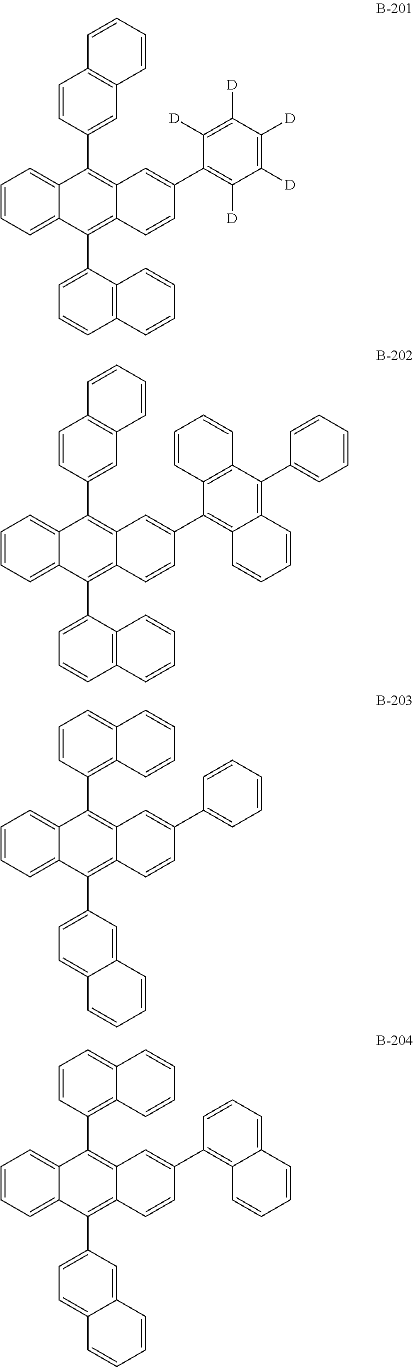

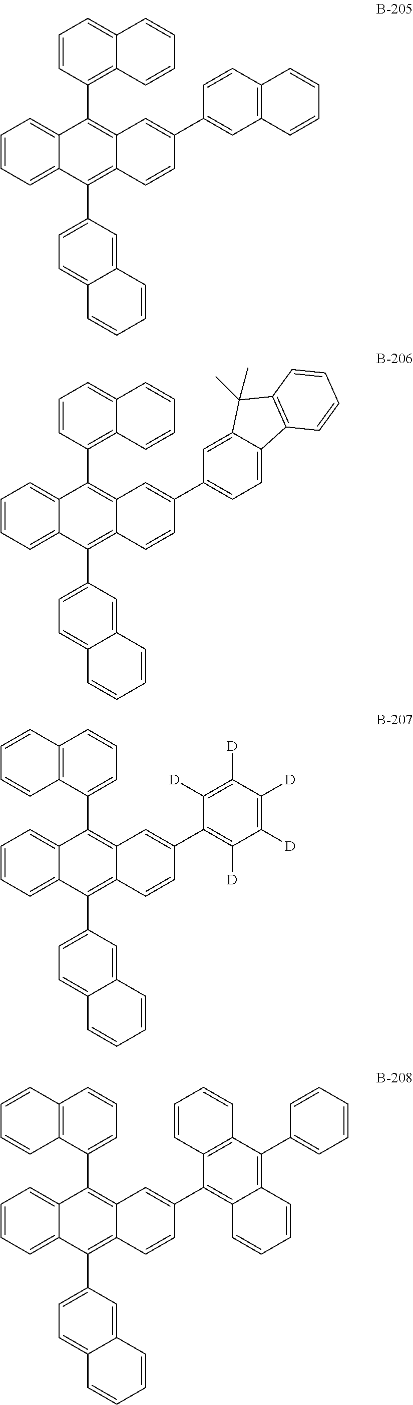

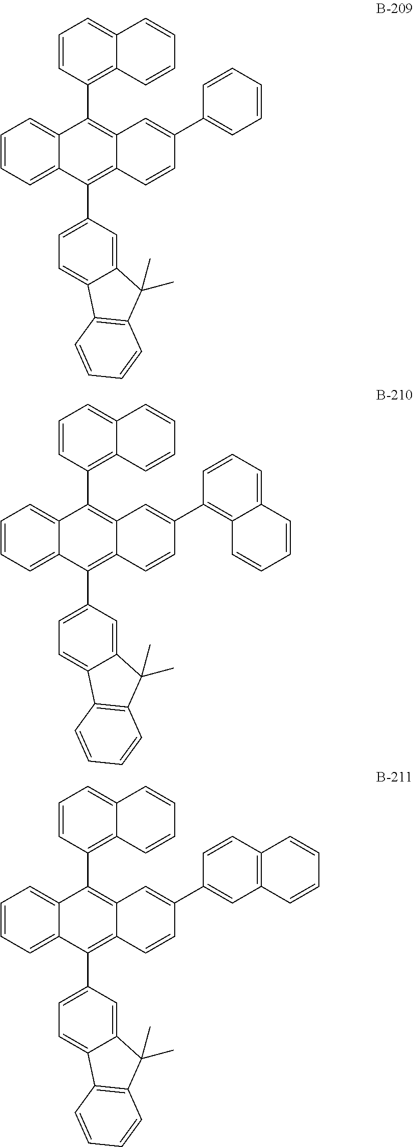

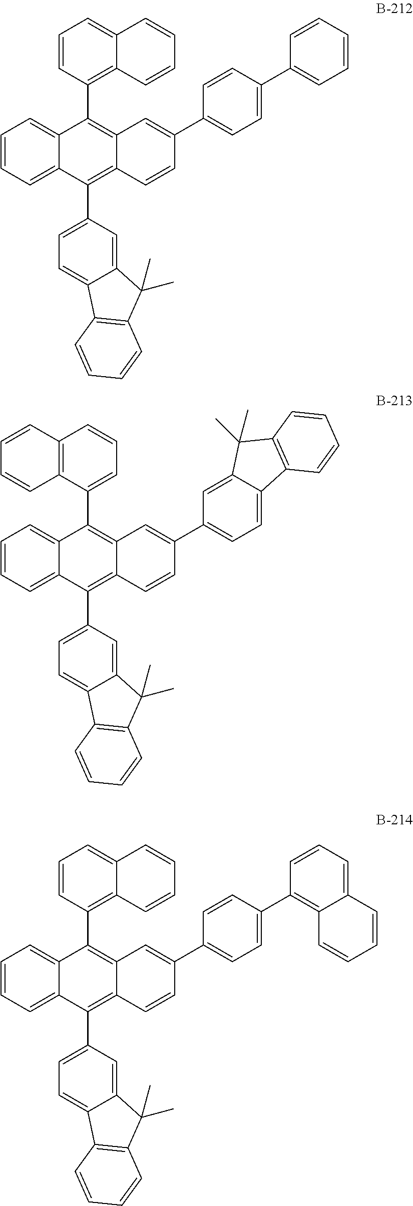

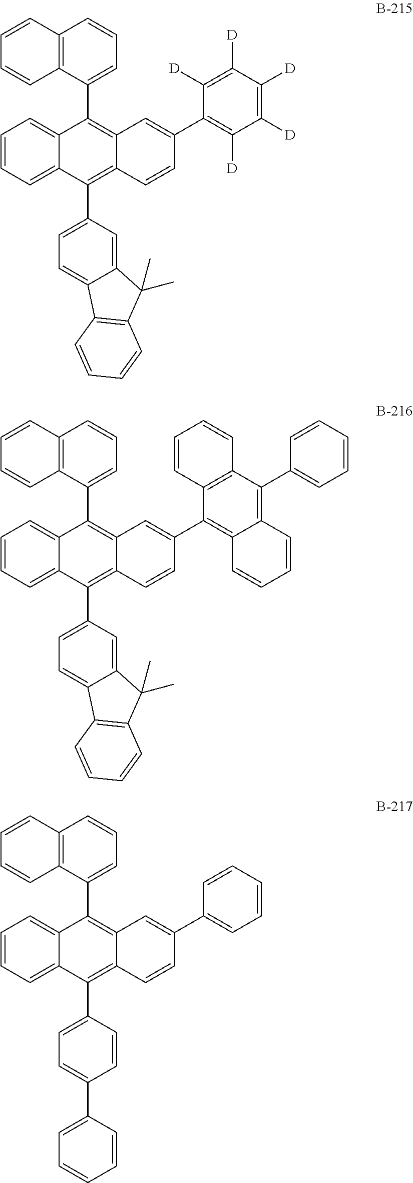

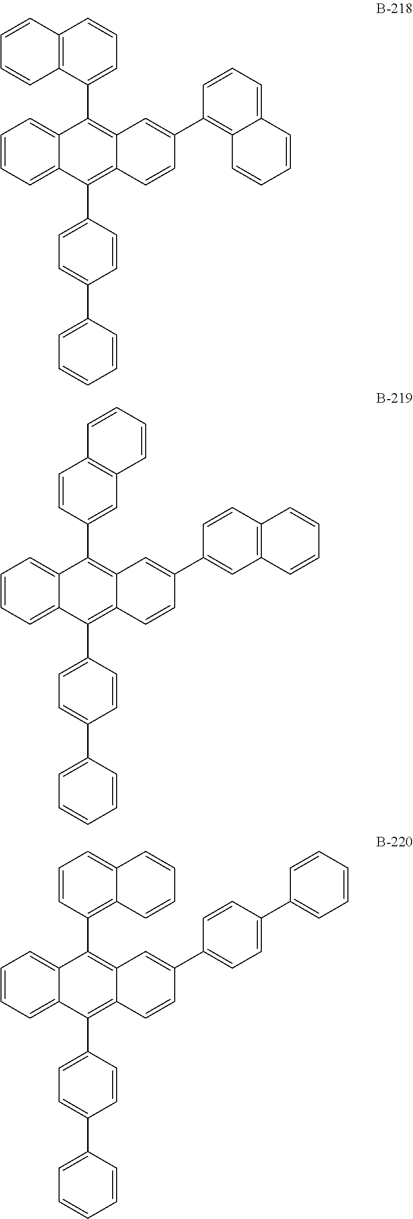

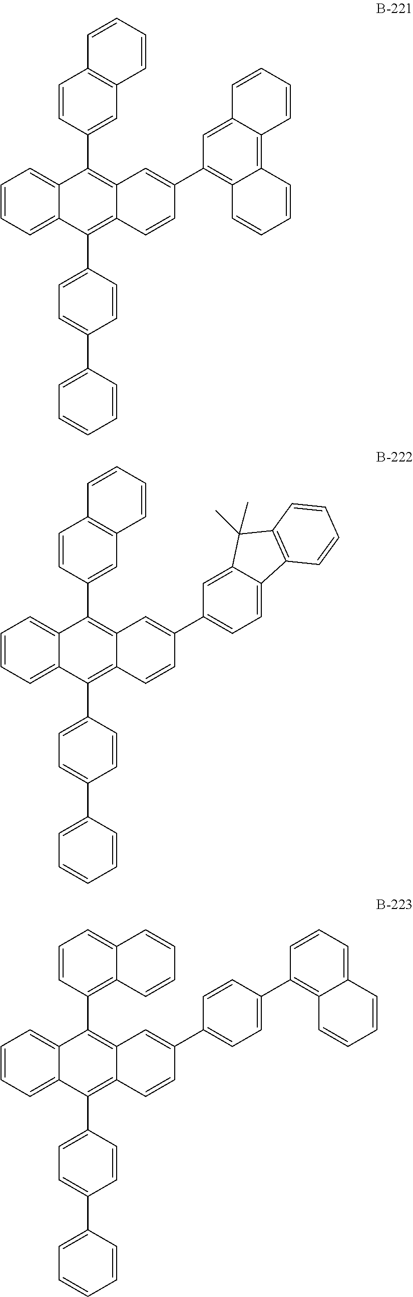

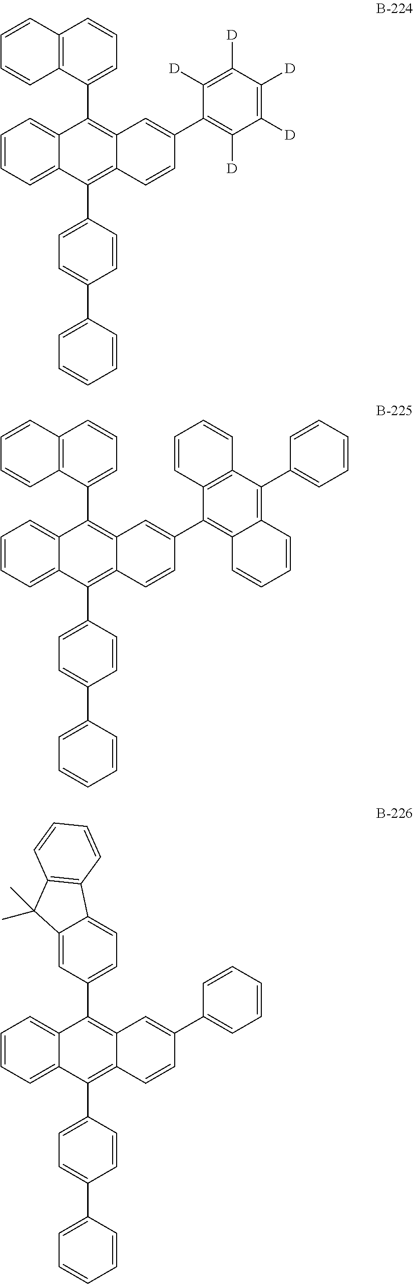

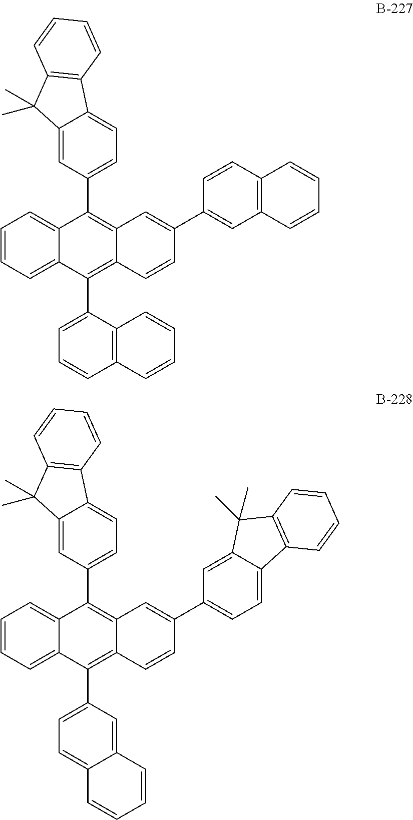

The compound of formula 1 of the present disclosure can be prepared by a synthetic method known to one skilled in the art.

According to one aspect of the present disclosure, the use as an electron buffering material of the compound represented by formula 1 is provided. Specifically, the use may be for the electron buffering material of an organic electroluminescent device.

According to another aspect of the present disclosure, an organic electroluminescent device is provided comprising a first electrode, a second electrode facing the first electrode, a light-emitting layer between the first electrode and the second electrode, and an electron transport zone and an electron buffering layer between the light-emitting layer and the second electrode, wherein the electron buffering layer comprises the compound represented by formula 1 above.

In the organic electroluminescent device, the light-emitting layer may comprise a host compound and a dopant compound.

LUMO energy level of the electron buffering layer may be about ±0.2 eV, preferably about ±0.1 eV based on LUMO energy level of the host compound. In the LUMO relationship for each layer, LUMO energy levels between the electron buffering layer and host compound are similar, thereby electrons are trapped between the electron buffering layer and the electron transport layer, which inhibits an injection of electrons to a light-emitting layer, and thus can cause an increase in driving voltage. However, an electron buffering layer comprising the compound represented by formula 1 can secure rapid electric current characteristics by the intermolecular stacking and the interation of interface with an electron buffering layer. Therefore, the organic electroluminescent device of the present disclosure can have low driving voltage and excellent luminous efficiency. Herein, specifically, LUMO energy level of an electron buffering layer may indicate LUMO energy level of the compound of formula 1 comprised in the electron buffering layer.

The host compound may be a phosphorescent host compound or a fluorescent host compound. Preferably, the host compound may be the compound represented by formula 1 above, and the host compound is the same as or different from the compound comprised to the electron buffering layer.

The dopant compound may be a phosphorescent dopant compound or a fluorescent dopant compound. The phosphorescent dopant compound is not limited, but may be preferably selected from metallated complex compounds of iridium (Ir), osmium (Os), copper (Cu) or platinum (Pt), more preferably selected from ortho-metallated complex compounds of iridium (Ir), osmium (Os), copper (Cu) or platinum (Pt), and even more preferably ortho-metallated iridium complex compounds. The fluorescent dopant compound is not limited, but may be preferably selected from styrylamine compounds, styryldiamine compounds, arylamine compounds, and aryldiamine compounds; and may be specifically a condensed polycyclic amine derivative represented by the following formula 5:

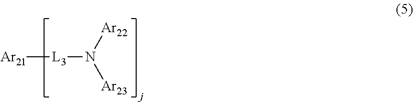

wherein Ar₂₁ represents a substituted or unsubstituted (C6-C50)aryl or styryl; L₃ represents a single bond, a substituted or unsubstituted (C6-C30)arylene, or a substituted or unsubstituted (3- to 30-membered)heteroarylene; Ar₂₂ and Ar₂₃, each independently, represent hydrogen, deuterium, a halogen, a substituted or unsubstituted (C1-C30)alkyl, a substituted or unsubstituted (C6-C30)aryl, or a substituted or unsubstituted (3- to 30-membered)heteroaryl, or may be linked to an adjacent substituent(s) to form a (C3-C30), mono- or polycyclic, alicyclic or aromatic ring whose carbon atom(s) may be replaced with at least one hetero atom selected from nitrogen, oxygen, and sulfur; j represents 1 or 2; and where j is 2, each of

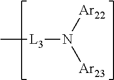

may be the same or different.

Specifically, the compound of formula 5 includes the following, but is not limited thereto:

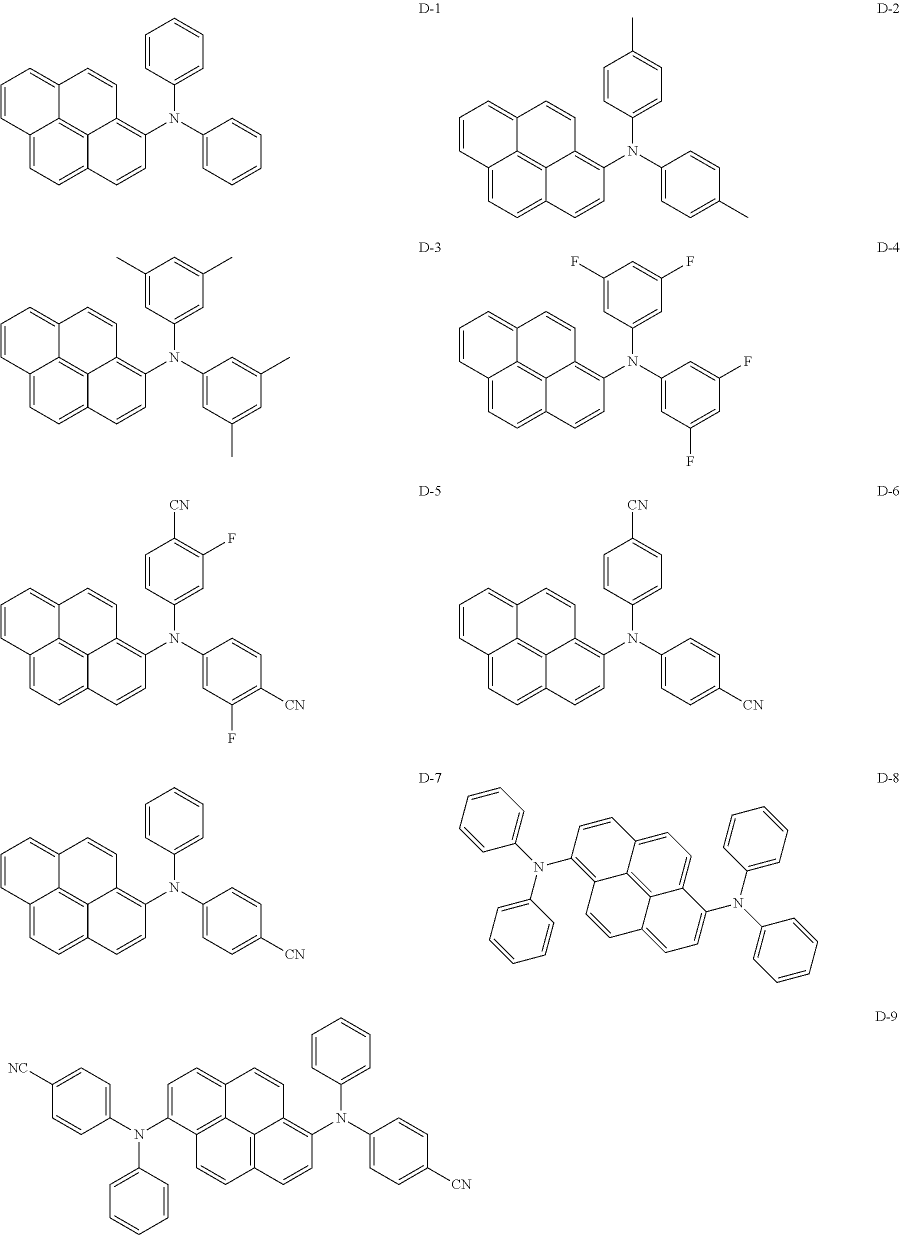

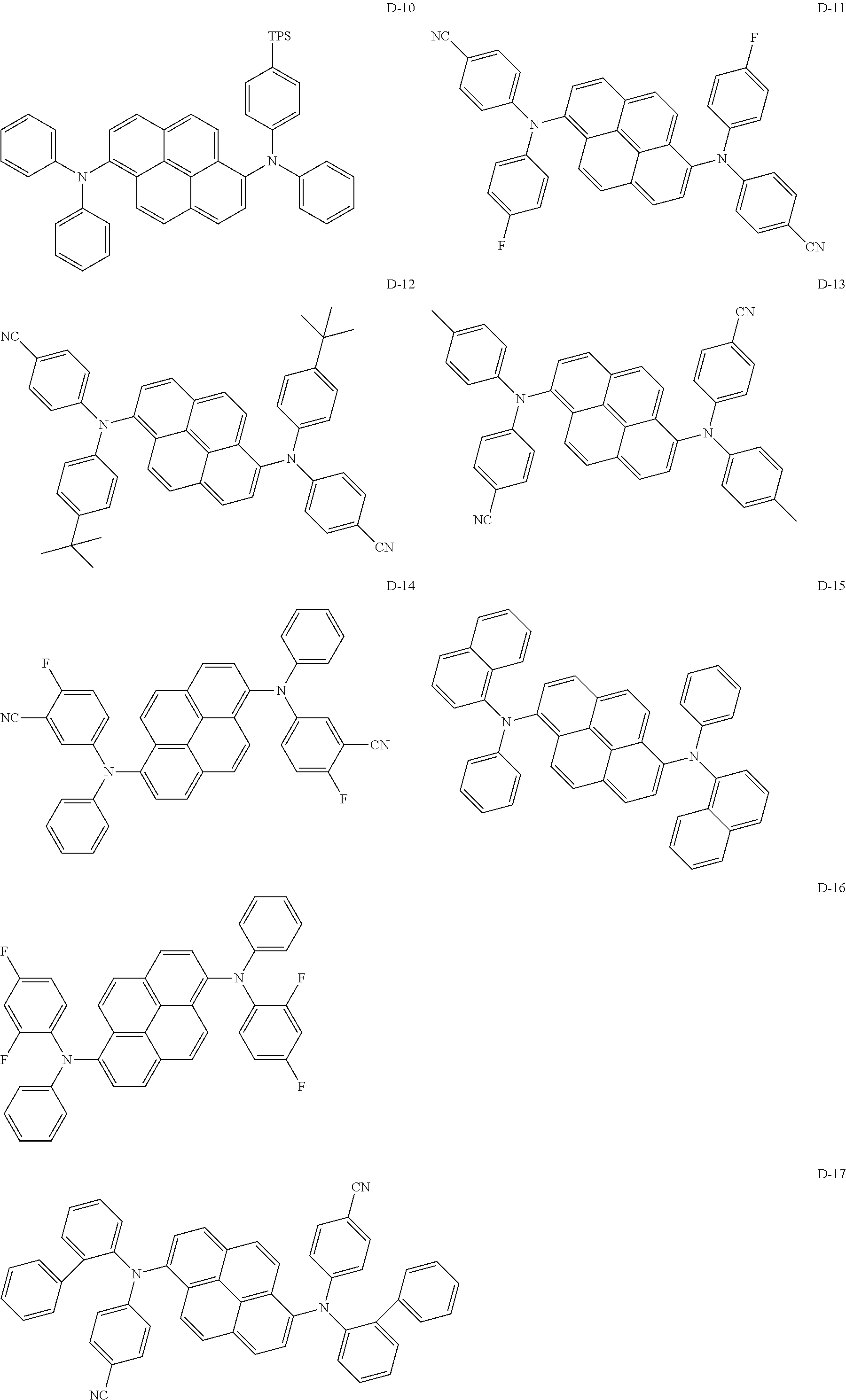

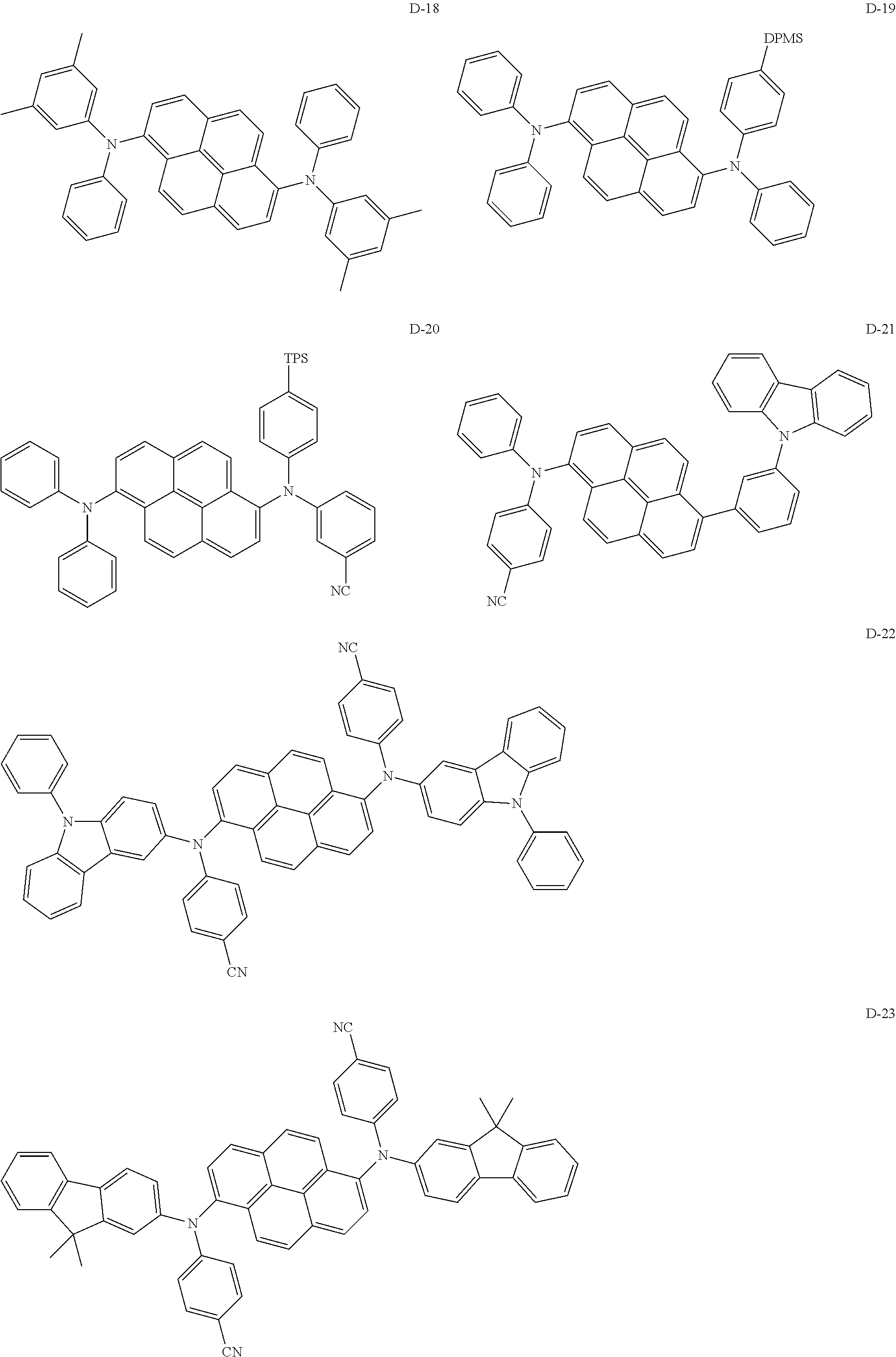

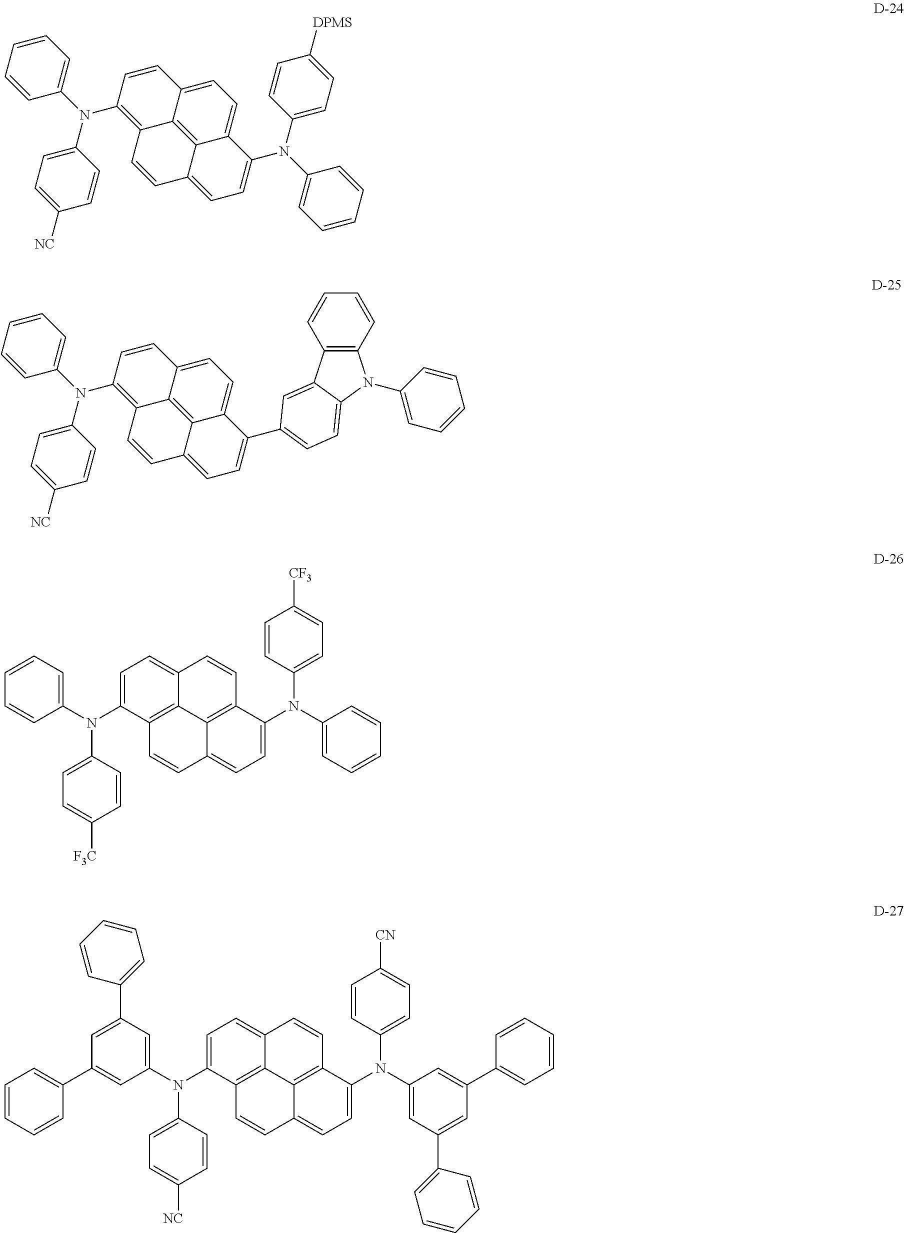

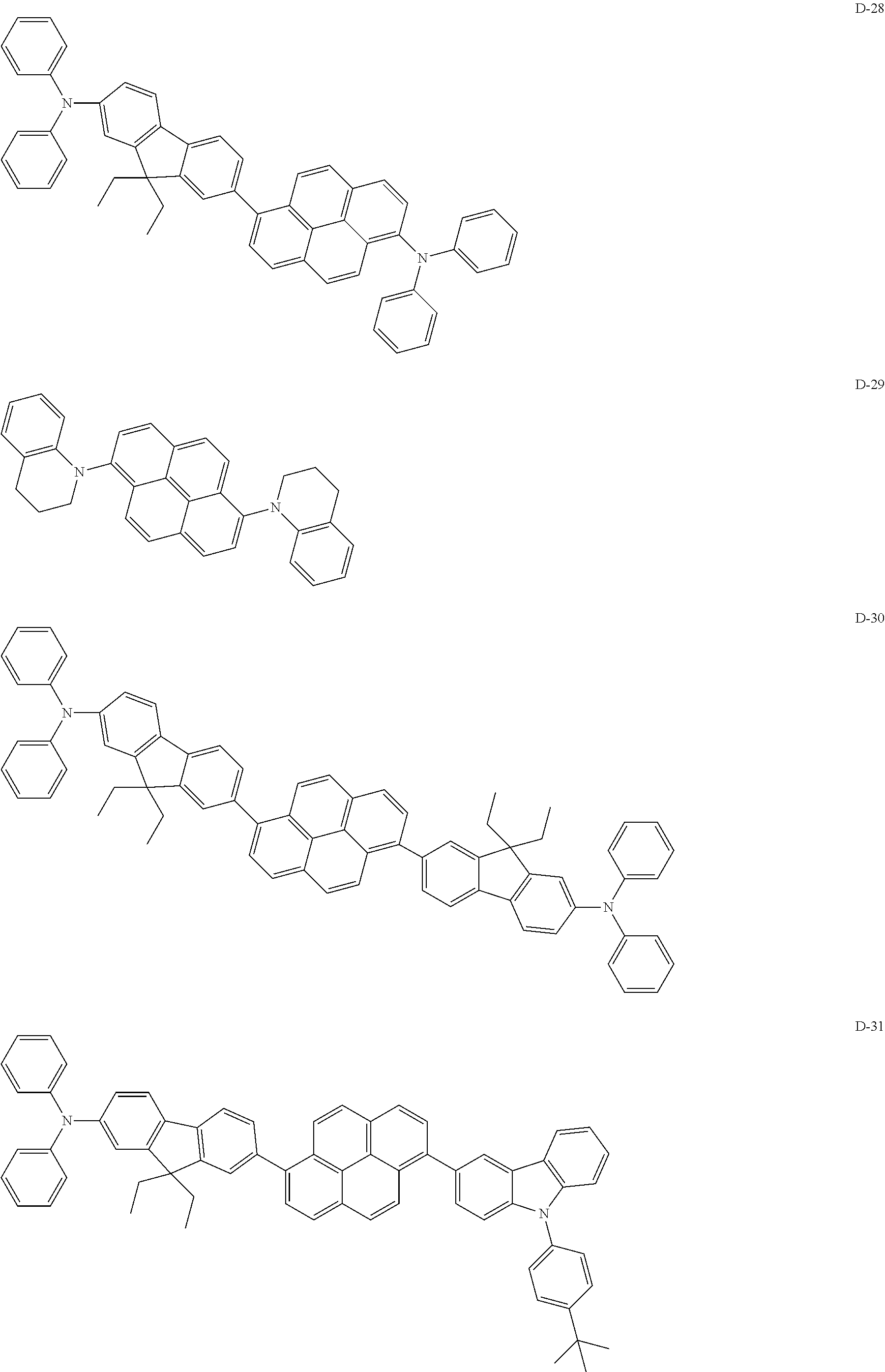 [SMILES: CCC1(CC)c2cc(-c3ccc4ccc5c(N(c6ccccc6)c6ccccc6)ccc6ccc3c4c65)ccc2-c2ccc(N(c3ccccc3)c3ccccc3)cc21 ; c1ccc2c(c1)CCCN2c1ccc2ccc3c(N4CCCc5ccccc54)ccc4ccc1c2c43 ; CCC1(CC)c2cc(-c3ccc4ccc5c(-c6ccc7c(c6)C(CC)(CC)c6cc(N(c8ccccc8)c8ccccc8)ccc6-7)ccc6ccc3c4c65)ccc2-c2ccc(N(c3ccccc3)c3ccccc3)cc21 ; CCC1(CC)c2cc(-c3ccc4ccc5c(-c6ccc7c(c6)c6ccccc6n7-c6ccc(C(C)(C)C)cc6)ccc6ccc3c4c65)ccc2-c2ccc(N(c3ccccc3)c3ccccc3)cc21]

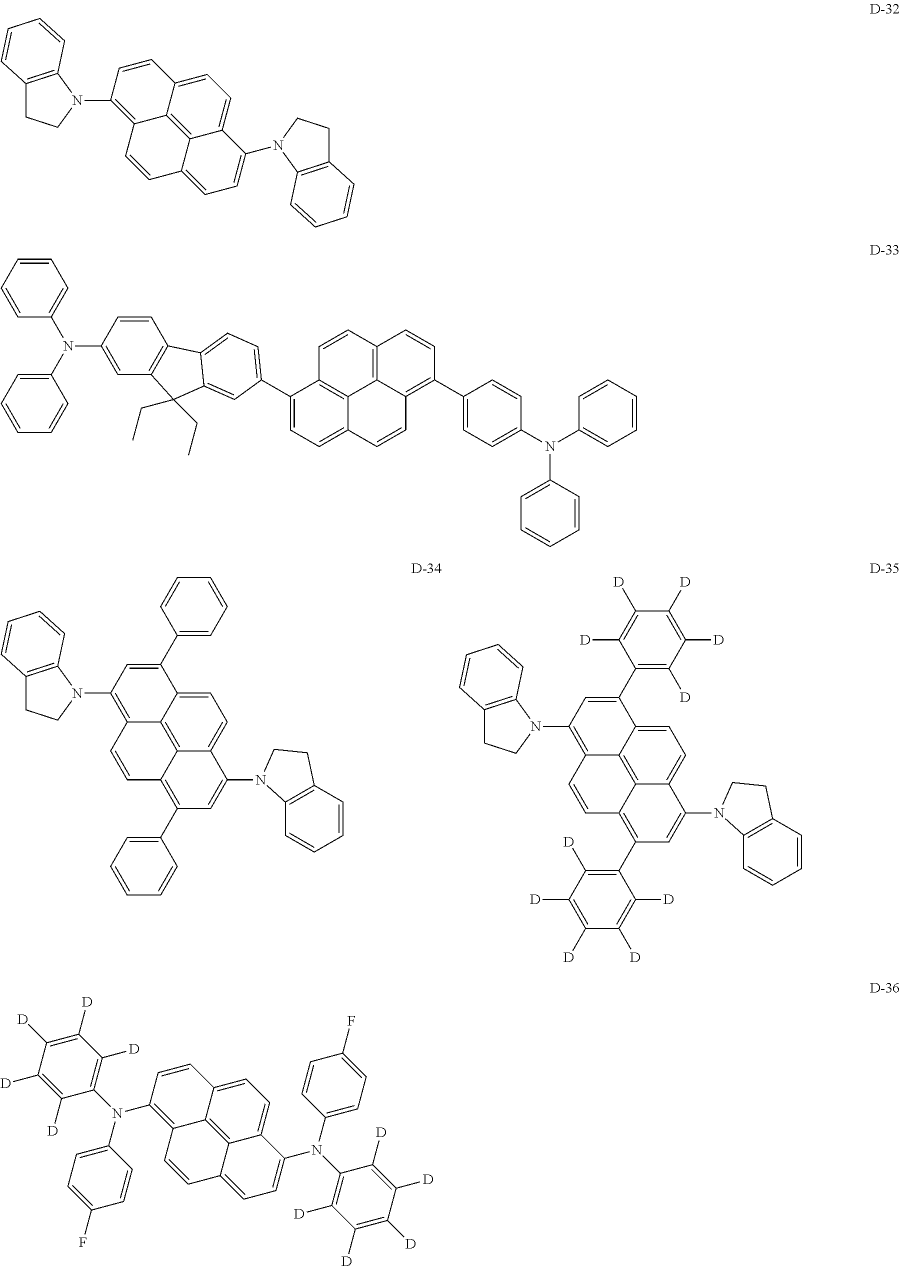

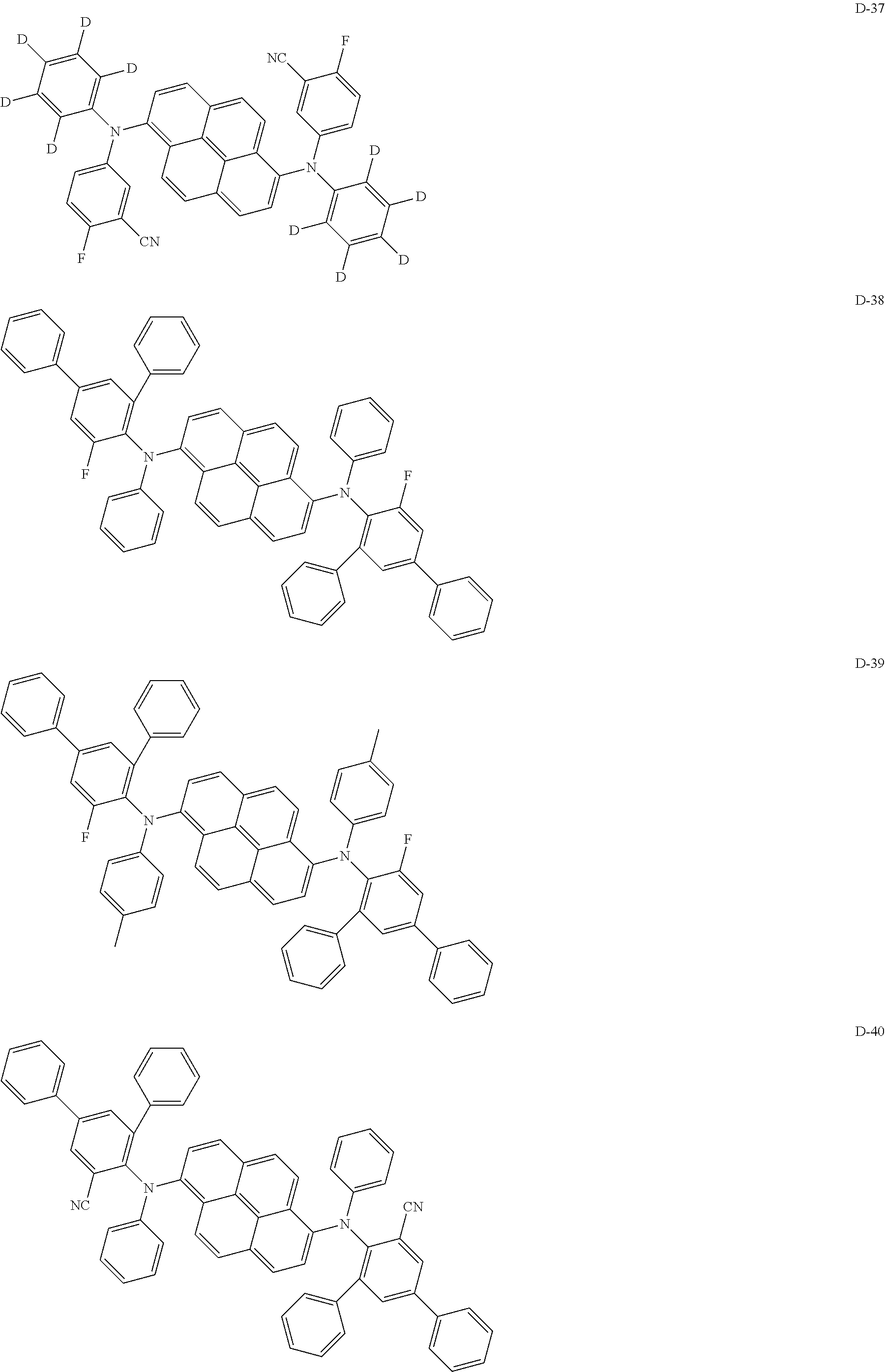 [SMILES: [2H]c1c([2H])c([2H])c(N(c2ccc(F)c(C#N)c2)c2ccc3ccc4c(N(c5ccc(F)c(C#N)c5)c5c([2H])c([2H])c([2H])c([2H])c5[2H])ccc5ccc2c3c54)c([2H])c1[2H] ; Fc1cc(-c2ccccc2)cc(-c2ccccc2)c1N(c1ccccc1)c1ccc2ccc3c(N(c4ccccc4)c4c(F)cc(-c5ccccc5)cc4-c4ccccc4)ccc4ccc1c2c43 ; Cc1ccc(N(c2c(F)cc(-c3ccccc3)cc2-c2ccccc2)c2ccc3ccc4c(N(c5ccc(C)cc5)c5c(F)cc(-c6ccccc6)cc5-c5ccccc5)ccc5ccc2c3c54)cc1 ; N#Cc1cc(-c2ccccc2)cc(-c2ccccc2)c1N(c1ccccc1)c1ccc2ccc3c(N(c4ccccc4)c4c(C#N)cc(-c5ccccc5)cc4-c4ccccc4)ccc4ccc1c2c43]

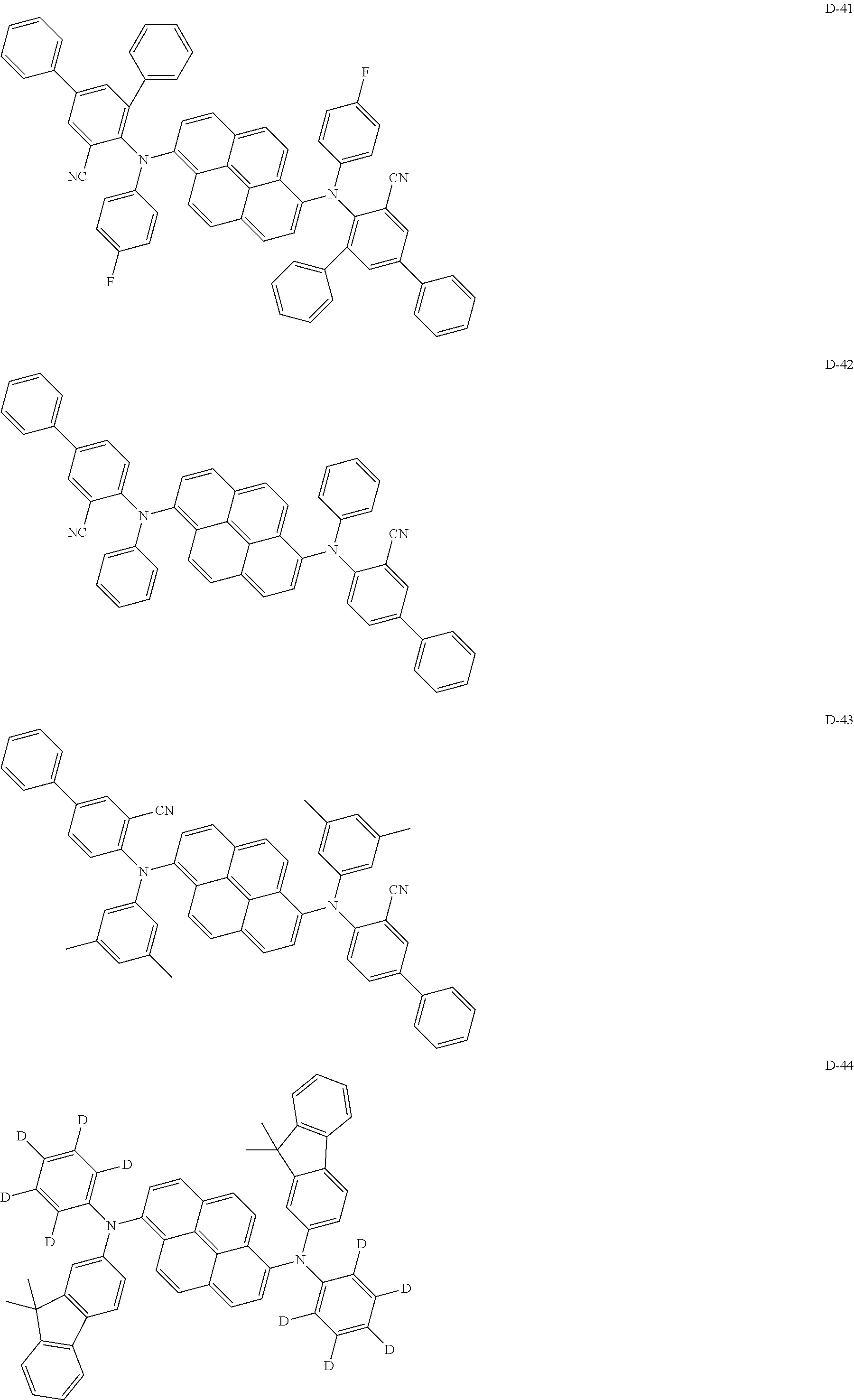 [SMILES: N#Cc1cc(-c2ccccc2)cc(-c2ccccc2)c1N(c1ccc(F)cc1)c1ccc2ccc3c(N(c4ccc(F)cc4)c4c(C#N)cc(-c5ccccc5)cc4-c4ccccc4)ccc4ccc1c2c43 ; N#Cc1cc(-c2ccccc2)ccc1N(c1ccccc1)c1ccc2ccc3c(N(c4ccccc4)c4ccc(-c5ccccc5)cc4C#N)ccc4ccc1c2c43 ; Cc1cc(C)cc(N(c2ccc(-c3ccccc3)cc2C#N)c2ccc3ccc4c(N(c5cc(C)cc(C)c5)c5ccc(-c6ccccc6)cc5C#N)ccc5ccc2c3c54)c1 ; [2H]c1c([2H])c([2H])c(N(c2ccc3c(c2)C(C)(C)c2ccccc2-3)c2ccc3ccc4c(N(c5ccc6c(c5)C(C)(C)c5ccccc5-6)c5c([2H])c([2H])c([2H])c([2H])c5[2H])ccc5ccc2c3c54)c([2H])c1[2H]]

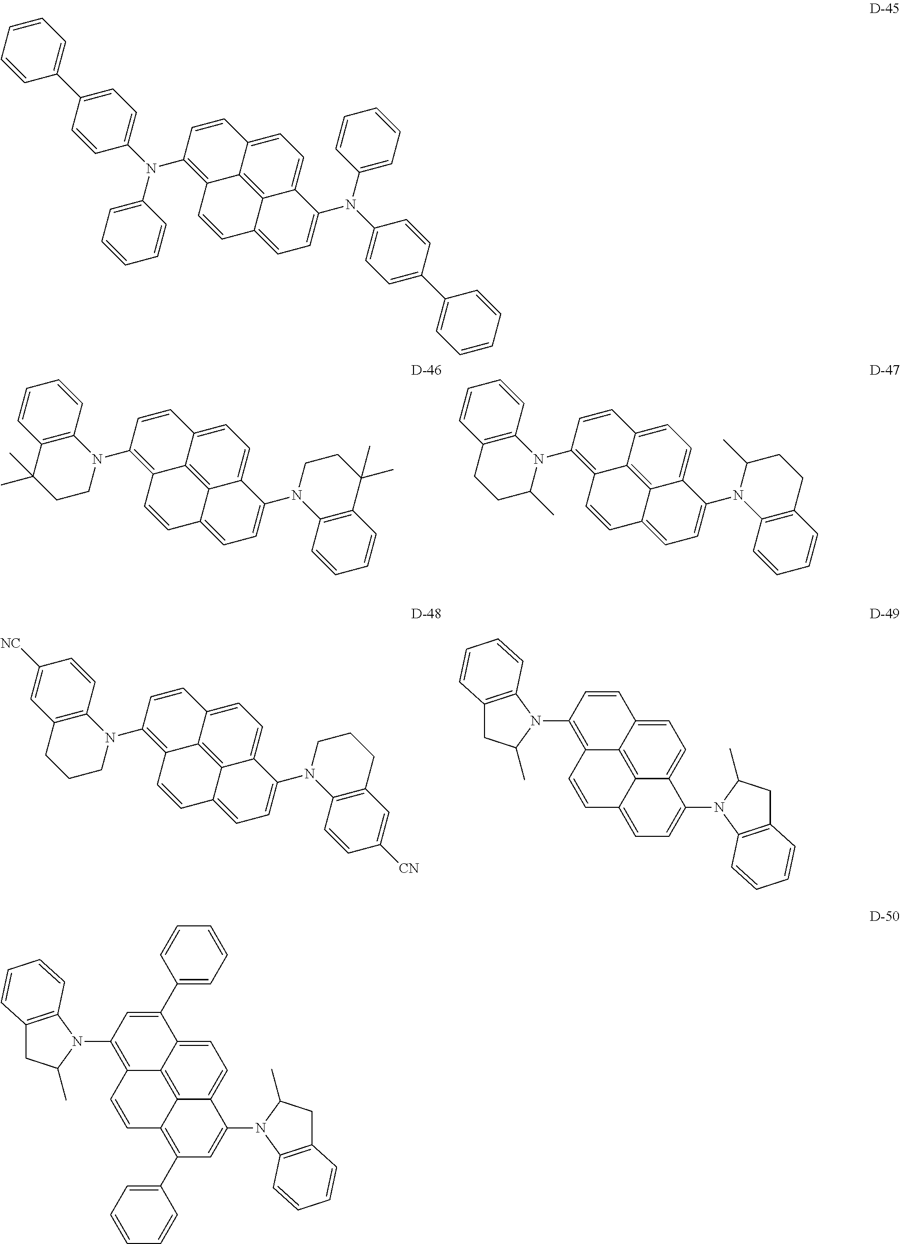

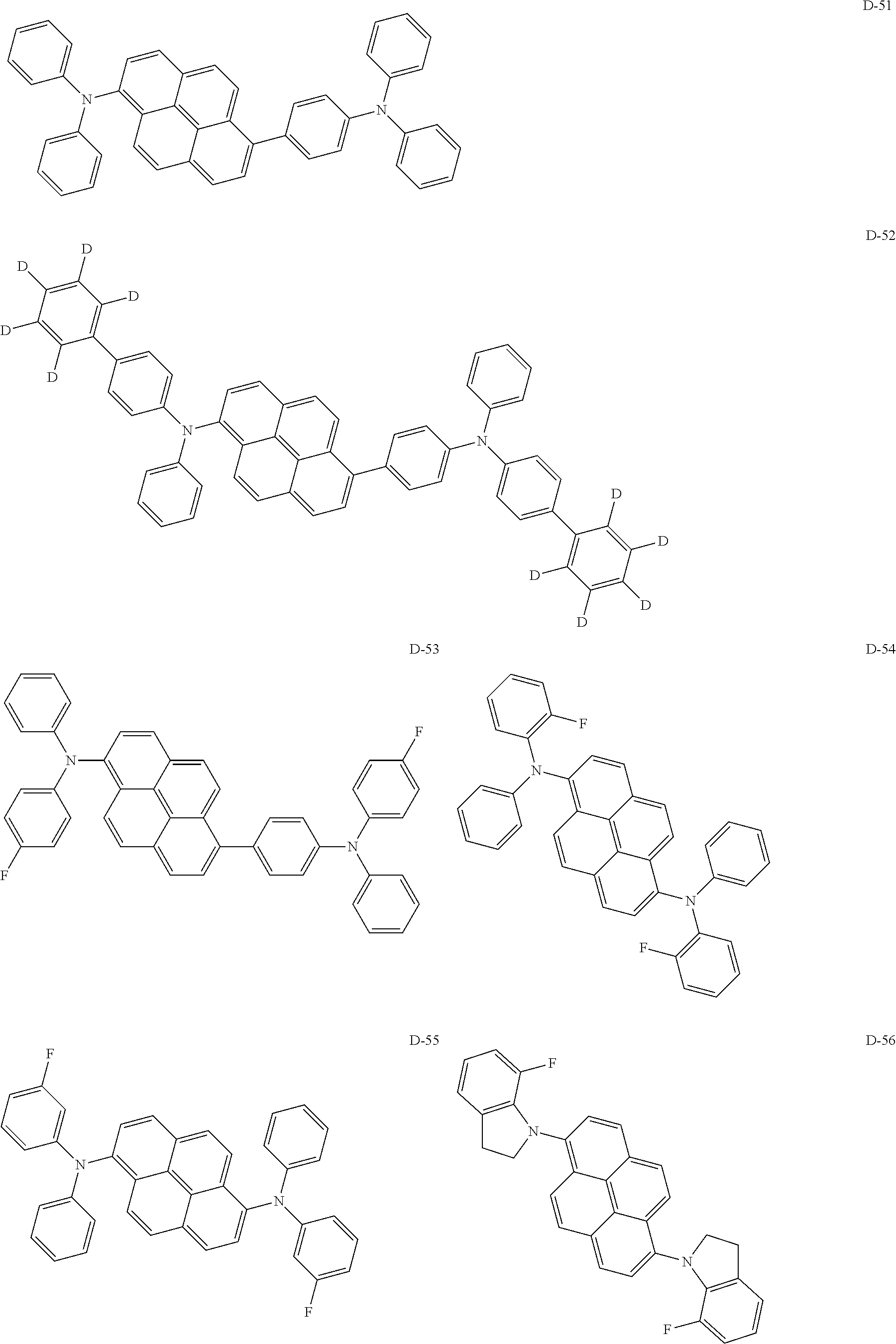

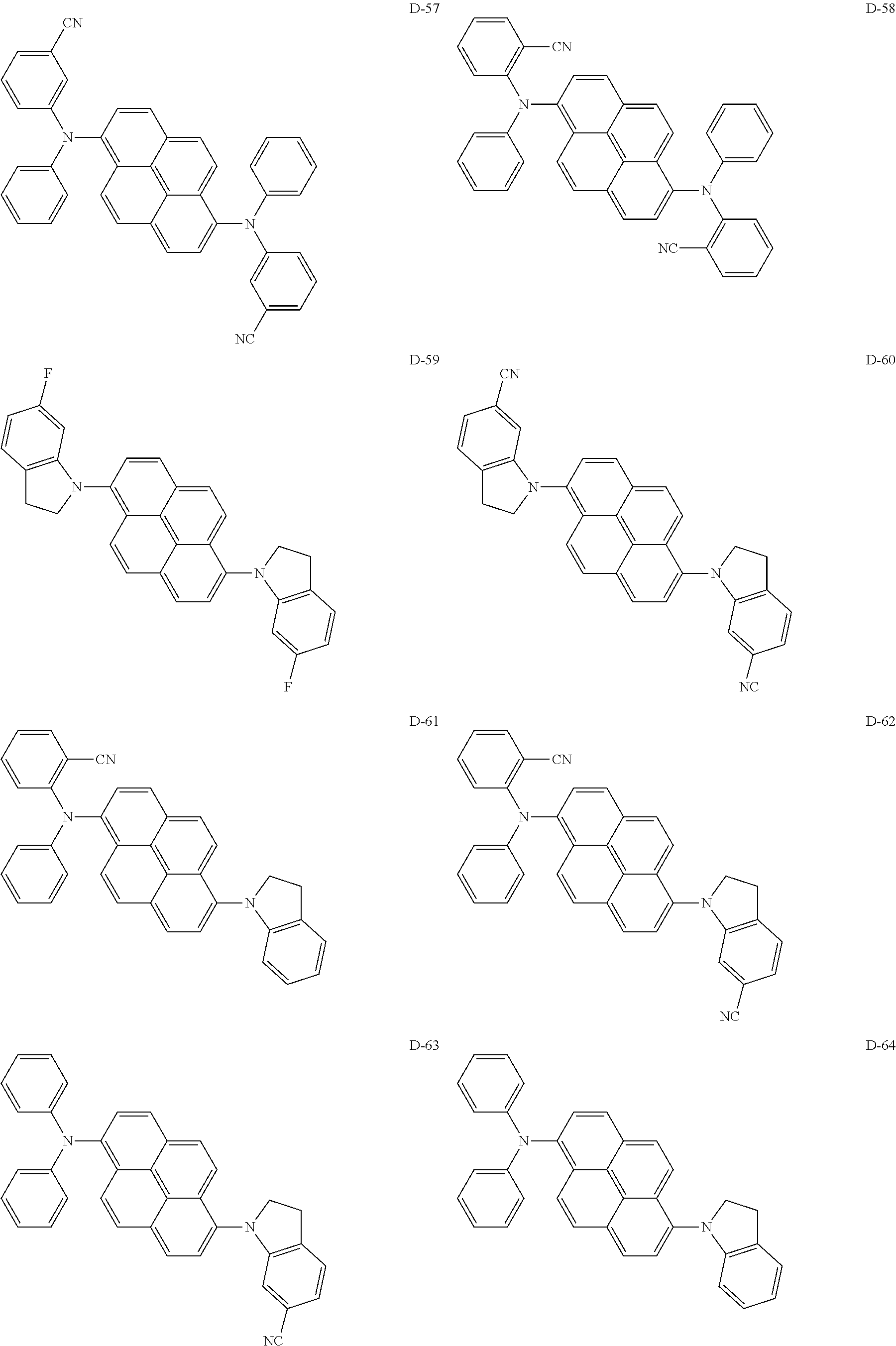

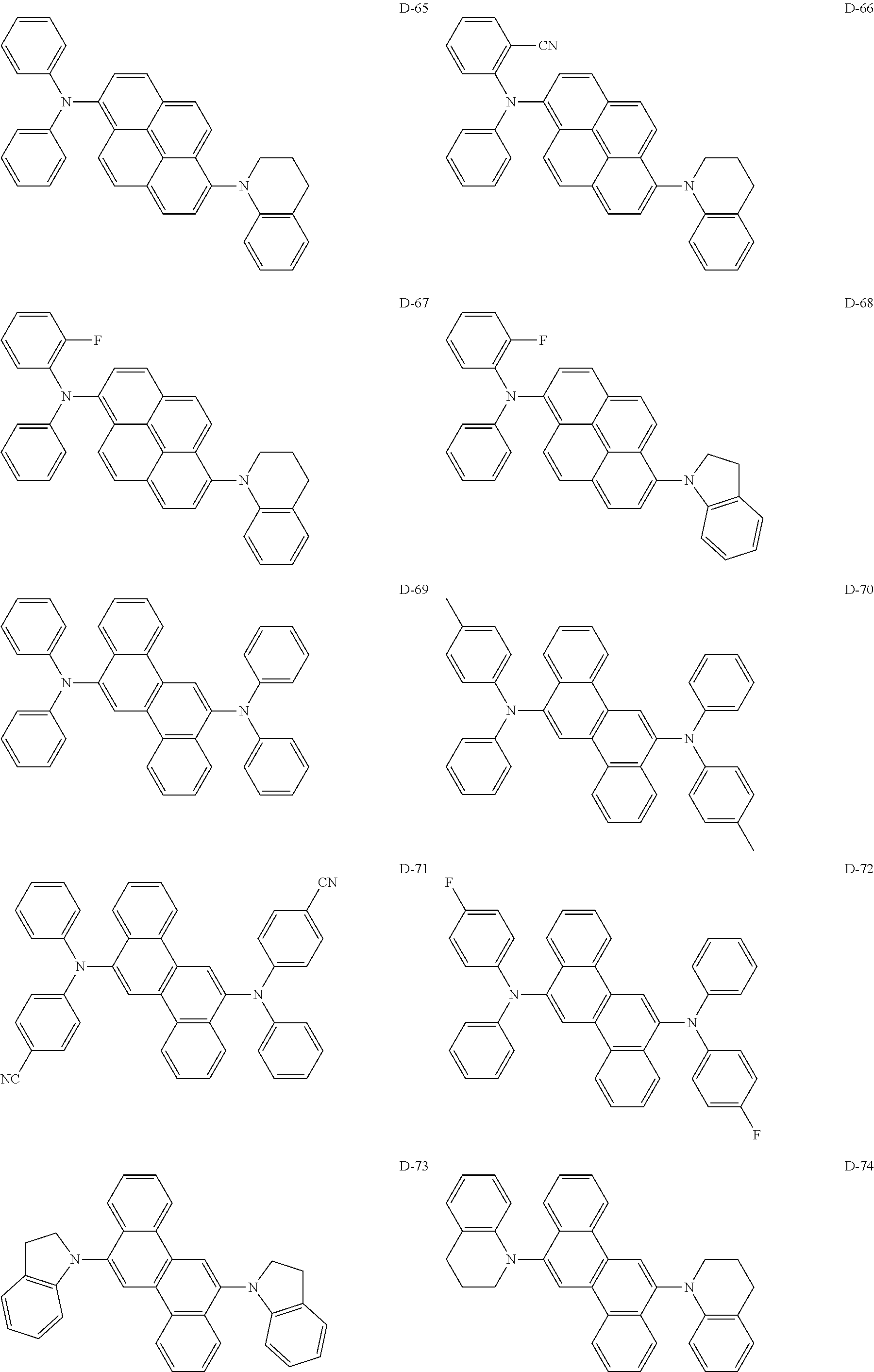

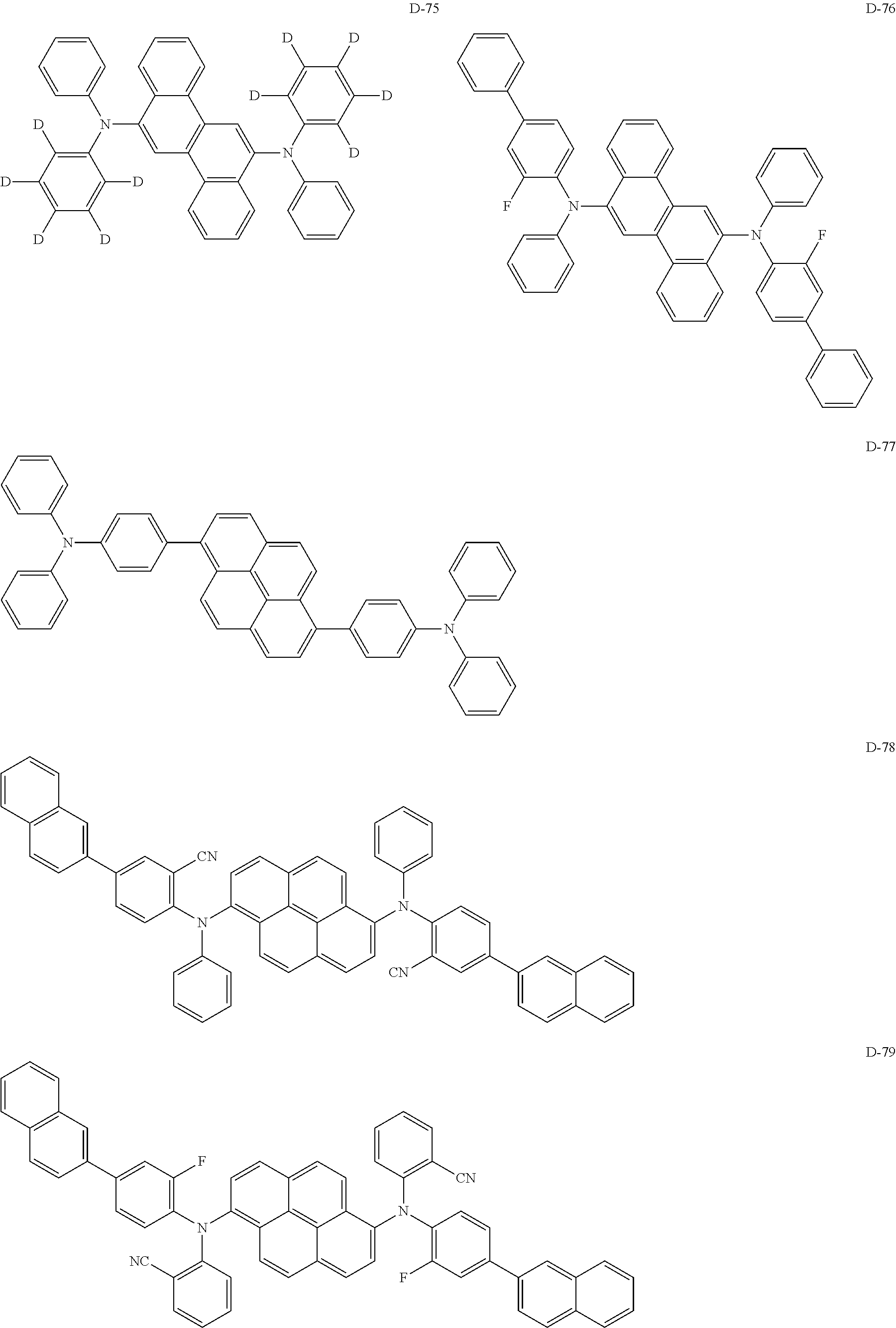

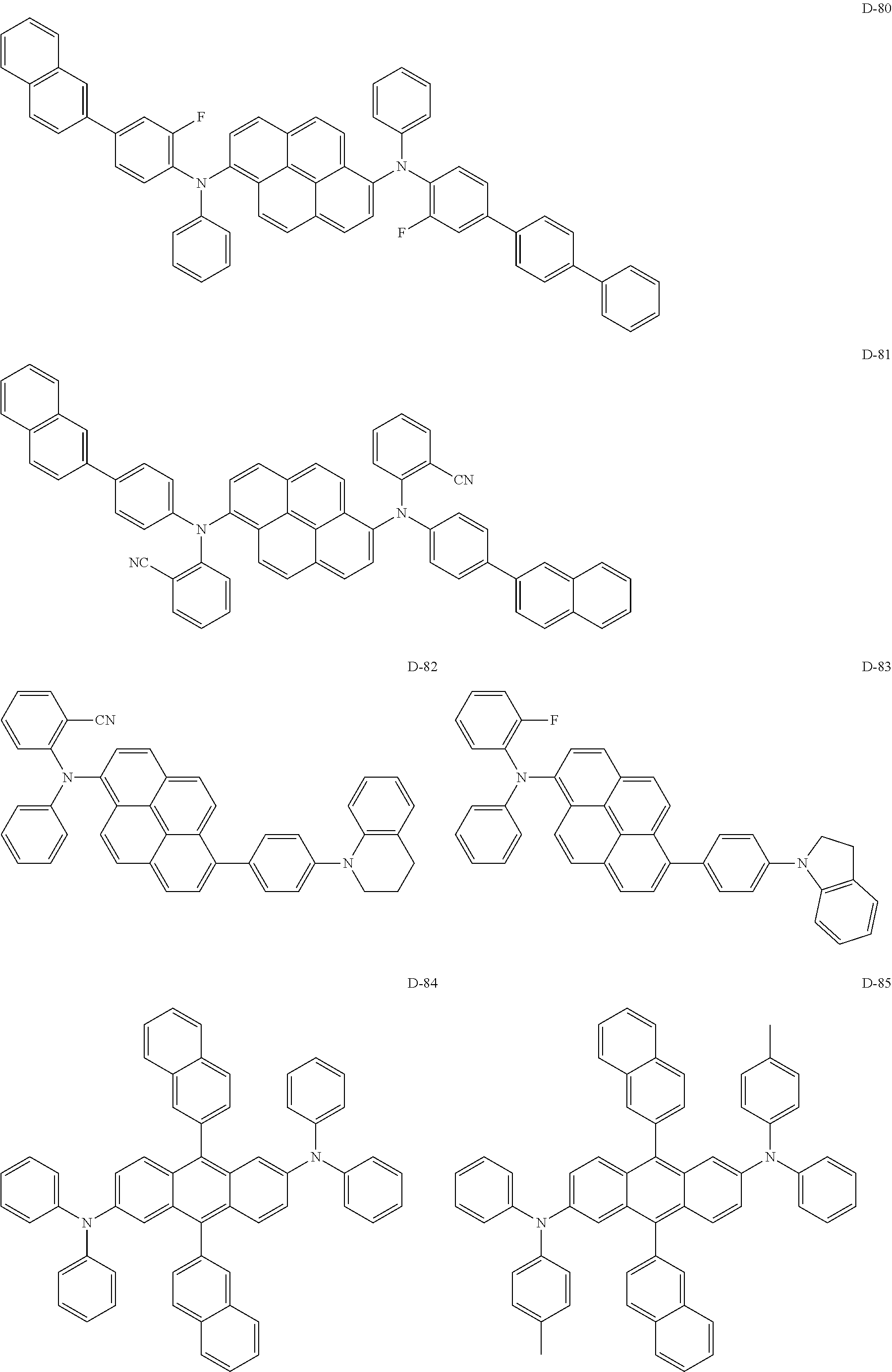

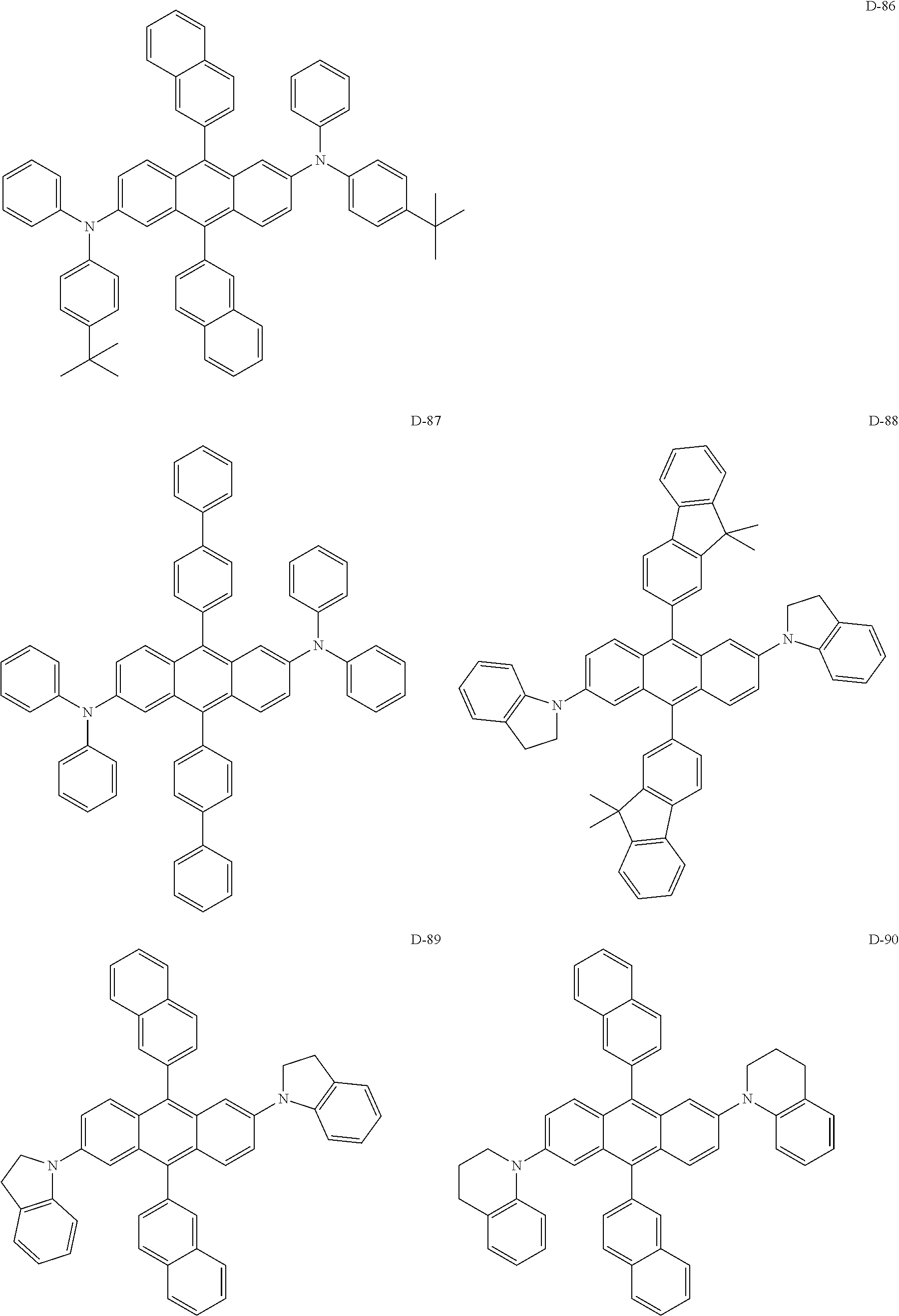

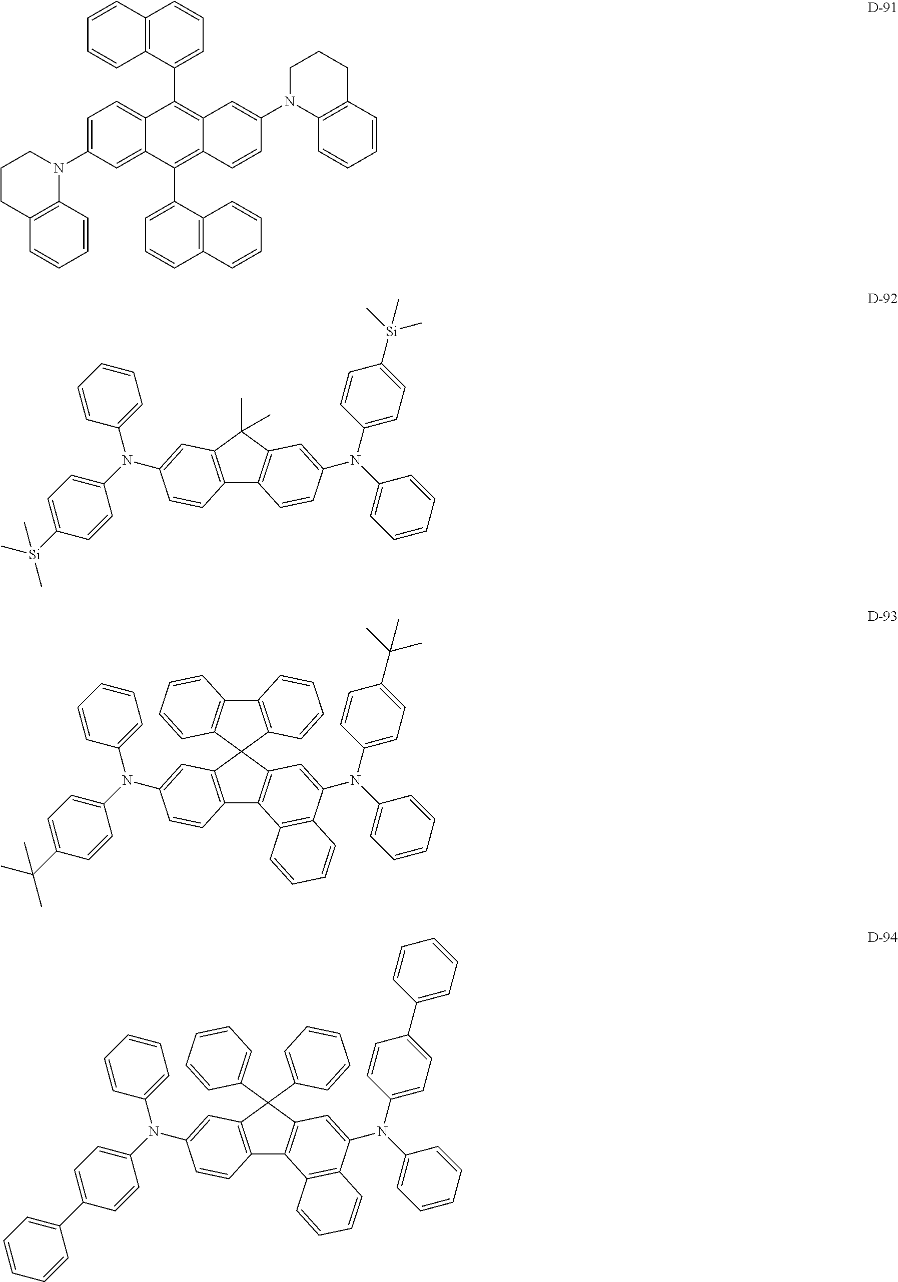

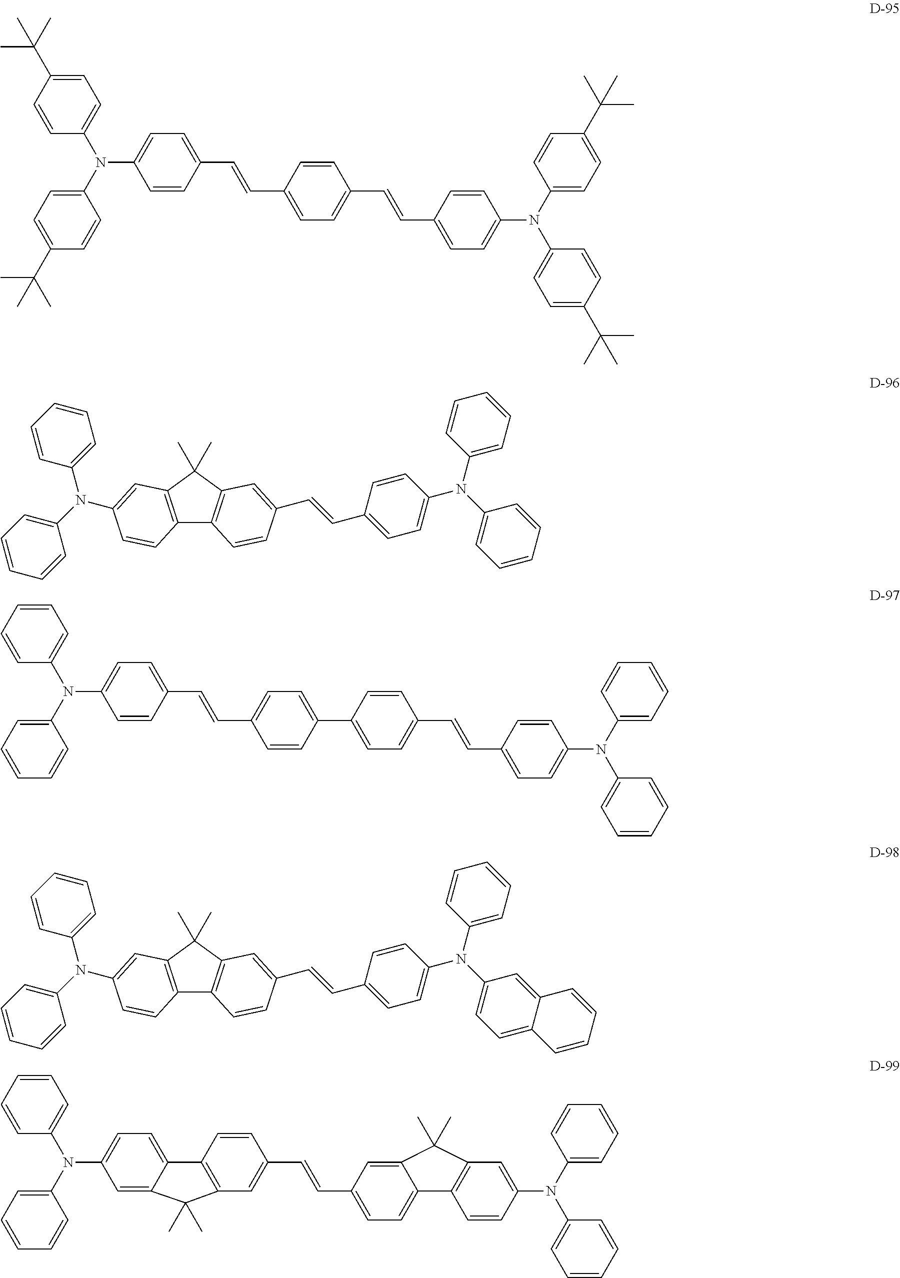

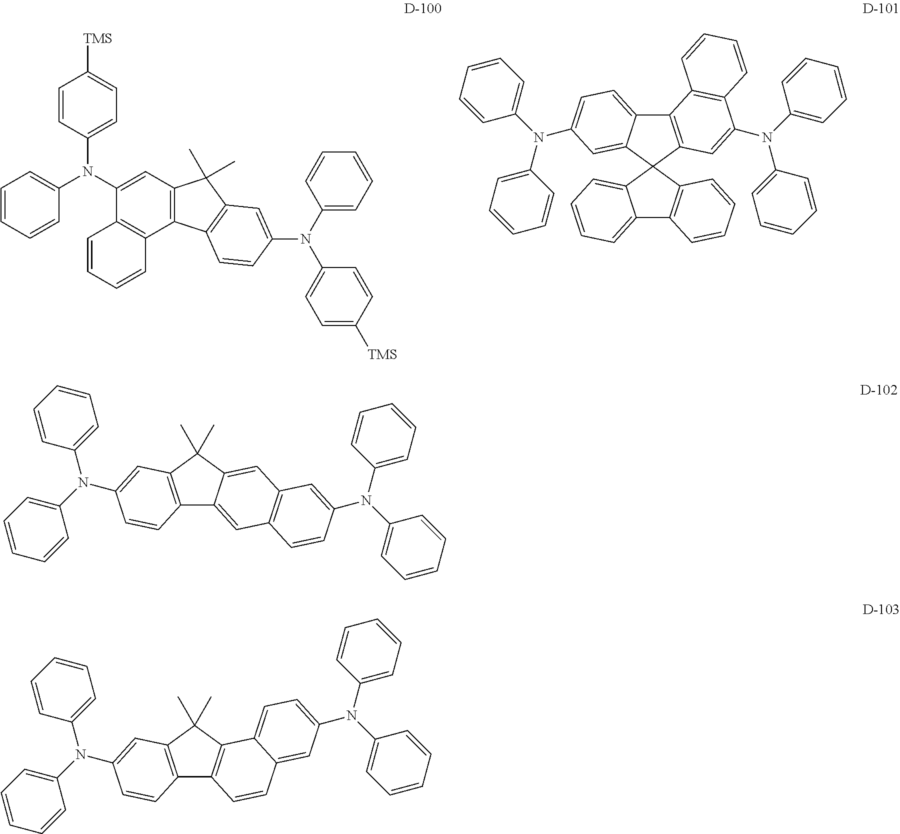 [SMILES: CC1(C)c2cc(N(c3ccccc3)c3ccc([Si](C)(C)C)cc3)ccc2-c2c1cc(N(c1ccccc1)c1ccc([Si](C)(C)C)cc1)c1ccccc21 ; c1ccc(N(c2ccccc2)c2ccc3c(c2)C2(c4ccccc4-c4ccccc42)c2cc(N(c4ccccc4)c4ccccc4)c4ccccc4c2-3)cc1 ; CC1(C)c2cc(N(c3ccccc3)c3ccccc3)ccc2-c2cc3ccc(N(c4ccccc4)c4ccccc4)cc3cc21 ; CC1(C)c2cc(N(c3ccccc3)c3ccccc3)ccc2-c2ccc3cc(N(c4ccccc4)c4ccccc4)ccc3c21]

In the organic electroluminescent device of the present disclosure, the electron transport zone indicates a zone transporting electrons from the second electrode to the light-emitting layer. The electron transport zone may comprise an electron transport compound, a reductive dopant, or a combination thereof.

The electron transport compound may be at least one selected from the group consisting of oxazole-based compounds, isoxazole-based compounds, triazole-based compounds, isothiazole-based compounds, oxadiazole-based compounds, thiadiazole-based compounds, perylene-based compounds, anthracene-based compounds, aluminum complexes, and gallium complexes. Specifically, the electron transport compound may be represented by the following formula 6.

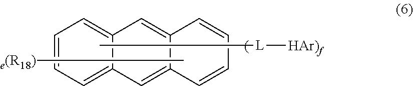

wherein

HAr is selected from the following formulae:

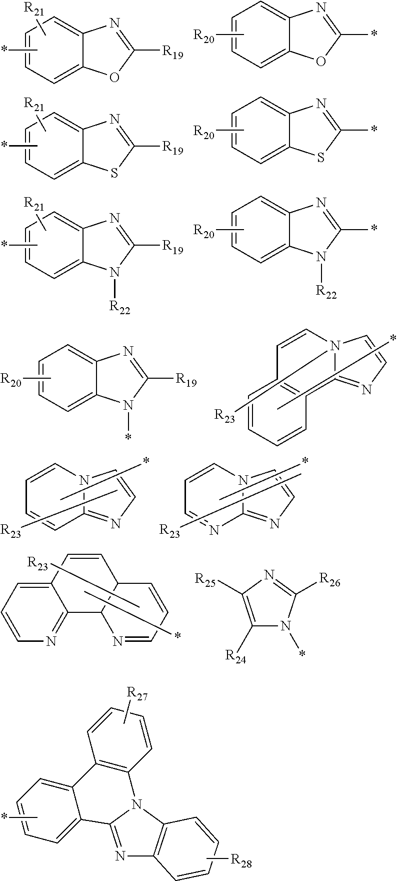

L represents a single bond, a substituted or unsubstituted (C6-C30)arylene, or a substituted or unsubstituted (5- to 30-membered)heteroarylene;

R₁₈ represents a substituted or unsubstituted a (C1-C30)alkyl, a substituted or unsubstituted (C6-C30)aryl, or a substituted or unsubstituted (5- to 30-membered)heteroaryl;

R₁₉ to R₂₈, each independently, represent hydrogen, deuterium, a halogen, a cyano, a carboxyl, a nitro, a hydroxyl, a substituted or unsubstituted (C1-C30)alkyl, a substituted or unsubstituted (C1-C30)alkoxy, a substituted or unsubstituted (C3-C30)cycloalkyl, a substituted or unsubstituted (C6-C30)aryl, or a substituted or unsubstituted (5- to 30-membered)heteroaryl; or are linked to an adjacent substituent(s) to form a substituted or unsubstituted, mono- or polycyclic, (C3-C30) alicyclic or aromatic ring, whose carbon atom(s) may be replaced with at least one hetero atom selected from nitrogen, oxygen, and sulfur;

e represents an integer of 0 to 3, where e is an integer of 2 or more, each of R₁₈ may be the same or different; and

f represents 1 or 2, where f is 2, each of (-L-HAr) may be the same or different.

Specifically, the compound represented by formula 6 may be represented by any one of the following formulae 6-1 to 6-4.

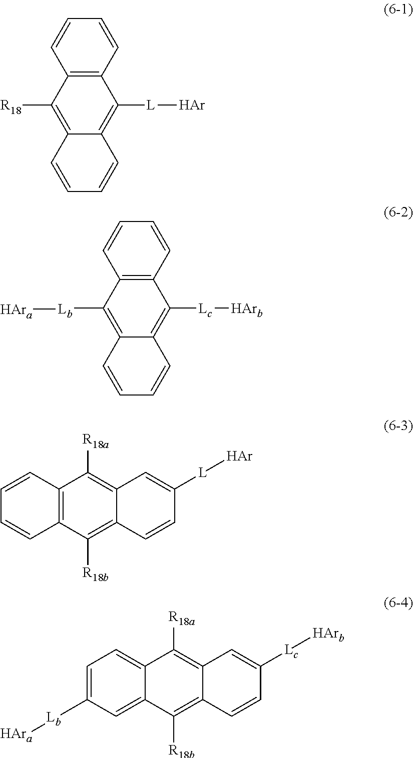

wherein, HArₐ and HAr_{b}, each independently, are as defined in HAr of formula 6, L_{b} and L_{c}, each independently, are as defined in L of formula 6, and R₁₈ₐ and R_{18b}, each independently, are as defined in R₁₈ of formula 6.

Specifically, the compound represented by formula 6 selected from the group consisting of the following compounds, but is not limited thereto:

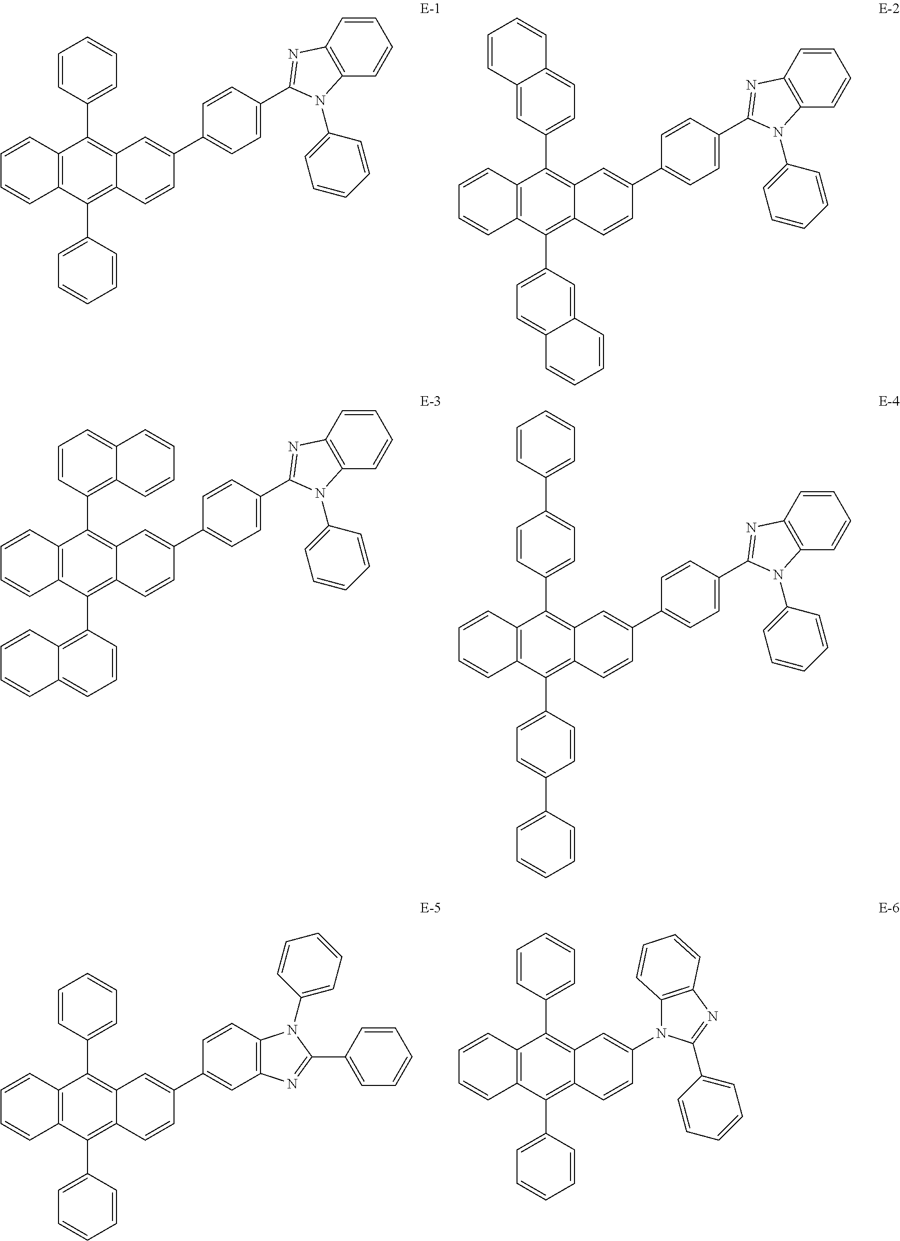

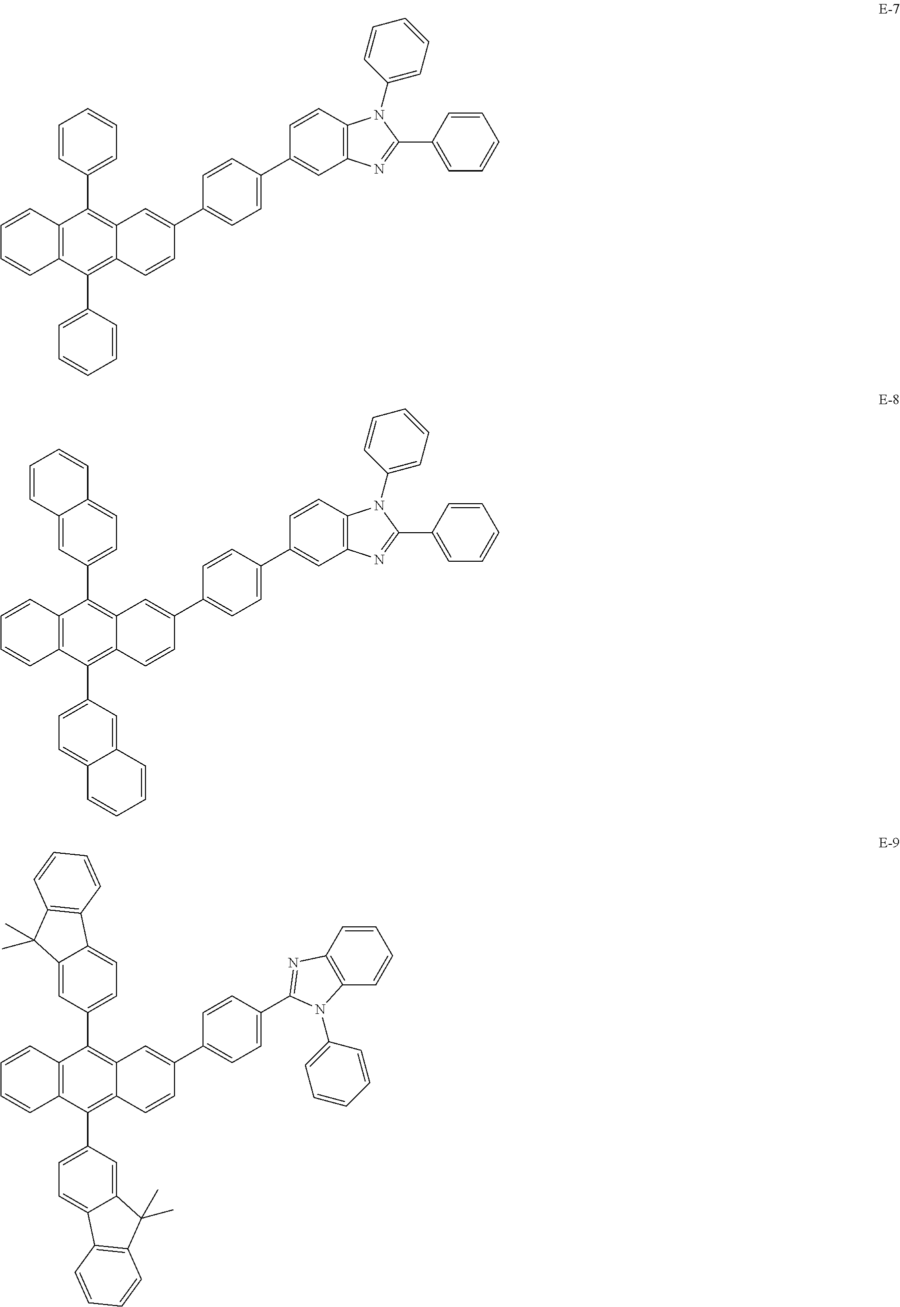 [SMILES: c1ccc(-c2c3ccccc3c(-c3ccccc3)c3cc(-c4ccc(-c5ccc6c(c5)nc(-c5ccccc5)n6-c5ccccc5)cc4)ccc23)cc1 ; c1ccc(-c2nc3cc(-c4ccc(-c5ccc6c(-c7ccc8ccccc8c7)c7ccccc7c(-c7ccc8ccccc8c7)c6c5)cc4)ccc3n2-c2ccccc2)cc1 ; CC1(C)c2ccccc2-c2ccc(-c3c4ccccc4c(-c4ccc5c(c4)C(C)(C)c4ccccc4-5)c4cc(-c5ccc(-c6nc7ccccc7n6-c6ccccc6)cc5)ccc34)cc21]

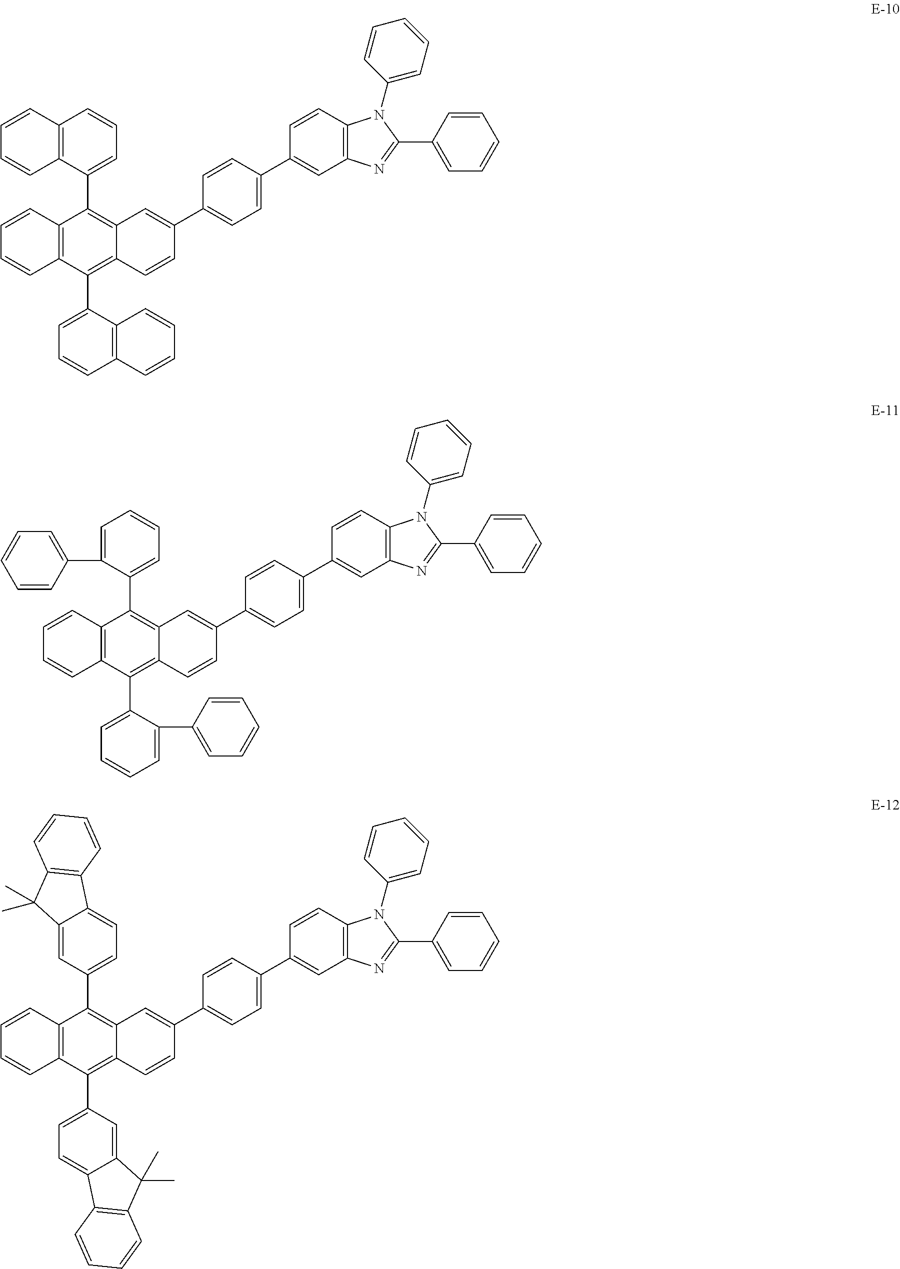 [SMILES: c1ccc(-c2nc3cc(-c4ccc(-c5ccc6c(-c7cccc8ccccc78)c7ccccc7c(-c7cccc8ccccc78)c6c5)cc4)ccc3n2-c2ccccc2)cc1 ; c1ccc(-c2ccccc2-c2c3ccccc3c(-c3ccccc3-c3ccccc3)c3cc(-c4ccc(-c5ccc6c(c5)nc(-c5ccccc5)n6-c5ccccc5)cc4)ccc23)cc1 ; CC1(C)c2ccccc2-c2ccc(-c3c4ccccc4c(-c4ccc5c(c4)C(C)(C)c4ccccc4-5)c4cc(-c5ccc(-c6ccc7c(c6)nc(-c6ccccc6)n7-c6ccccc6)cc5)ccc34)cc21]

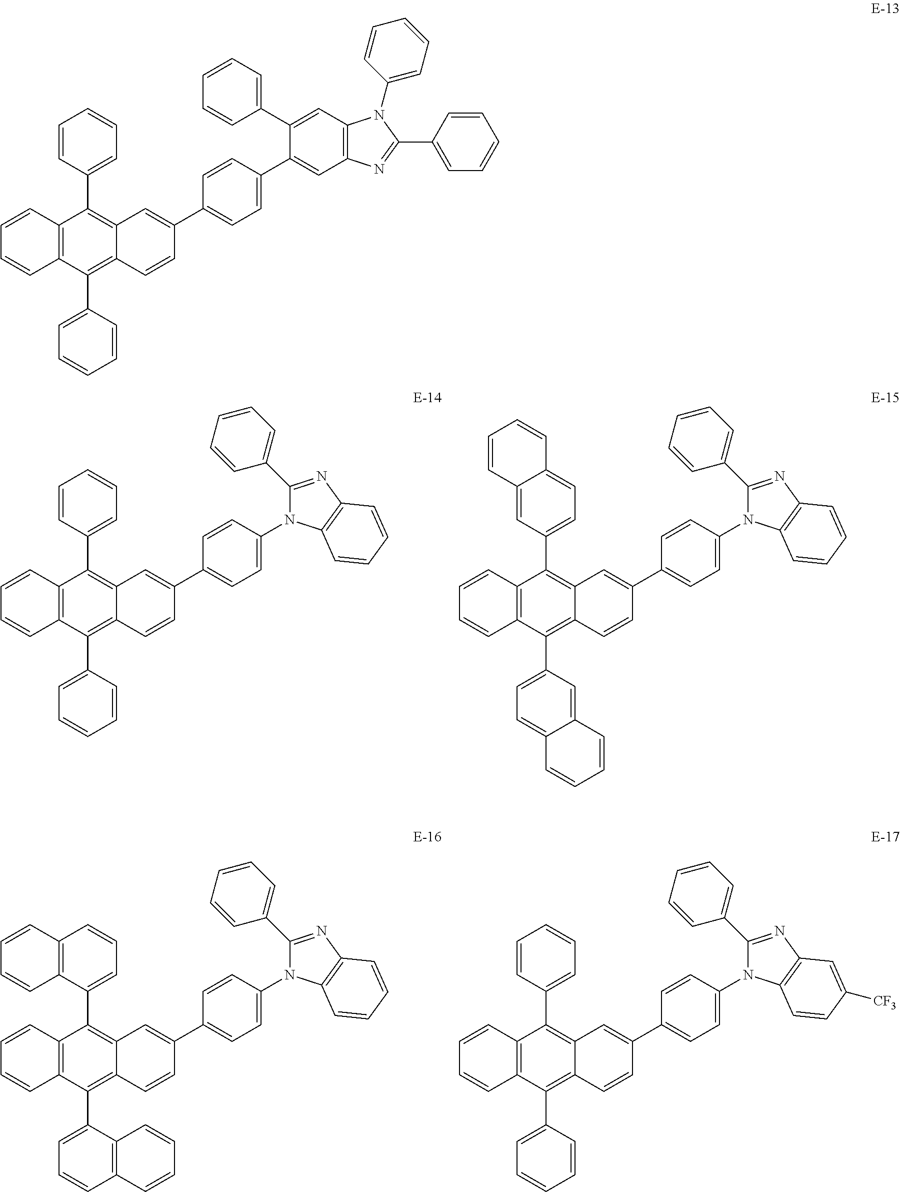 [SMILES: c1ccc(-c2cc3c(cc2-c2ccc(-c4ccc5c(-c6ccccc6)c6ccccc6c(-c6ccccc6)c5c4)cc2)nc(-c2ccccc2)n3-c2ccccc2)cc1 ; c1ccc(-c2c3ccccc3c(-c3ccccc3)c3cc(-c4ccc(-n5c(-c6ccccc6)nc6ccccc65)cc4)ccc23)cc1 ; c1ccc(-c2nc3ccccc3n2-c2ccc(-c3ccc4c(-c5ccc6ccccc6c5)c5ccccc5c(-c5ccc6ccccc6c5)c4c3)cc2)cc1 ; c1ccc(-c2nc3ccccc3n2-c2ccc(-c3ccc4c(-c5cccc6ccccc56)c5ccccc5c(-c5cccc6ccccc56)c4c3)cc2)cc1 ; FC(F)(F)c1ccc2c(c1)nc(-c1ccccc1)n2-c1ccc(-c2ccc3c(-c4ccccc4)c4ccccc4c(-c4ccccc4)c3c2)cc1]

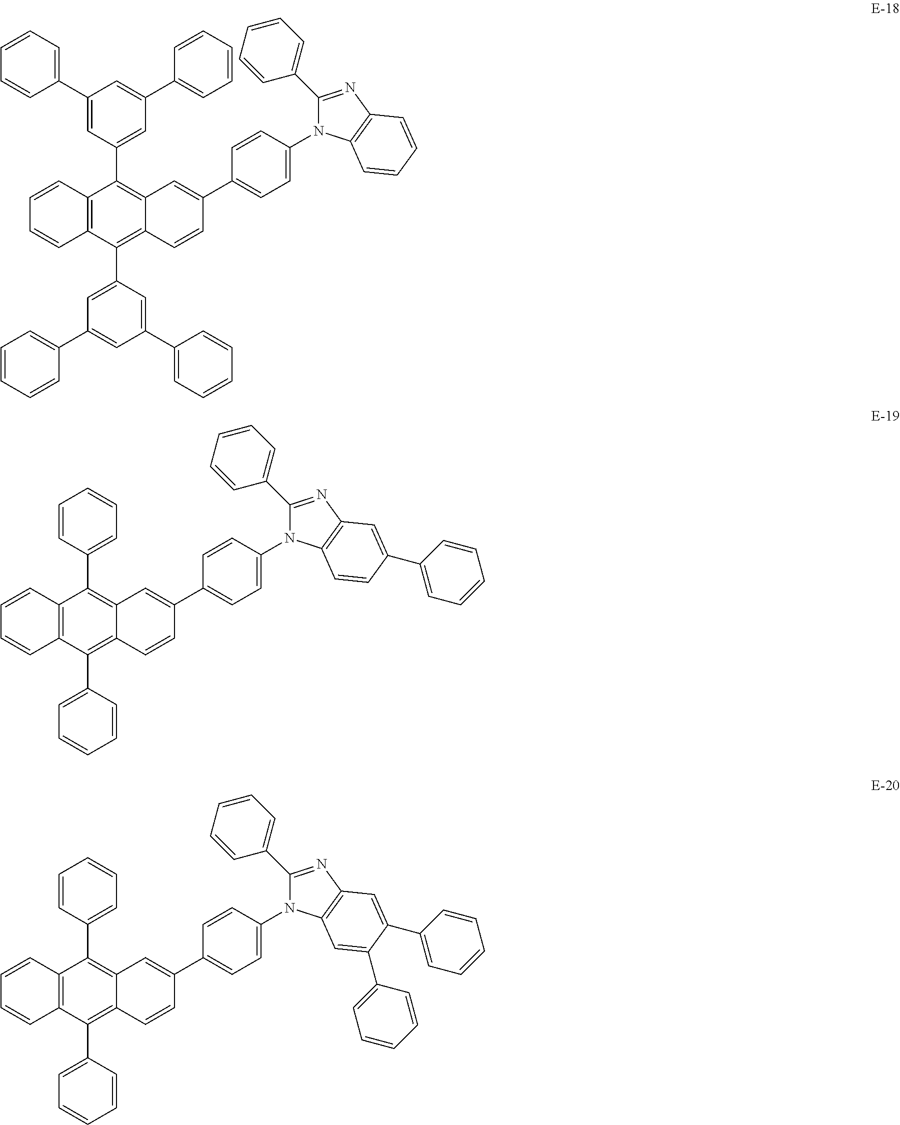

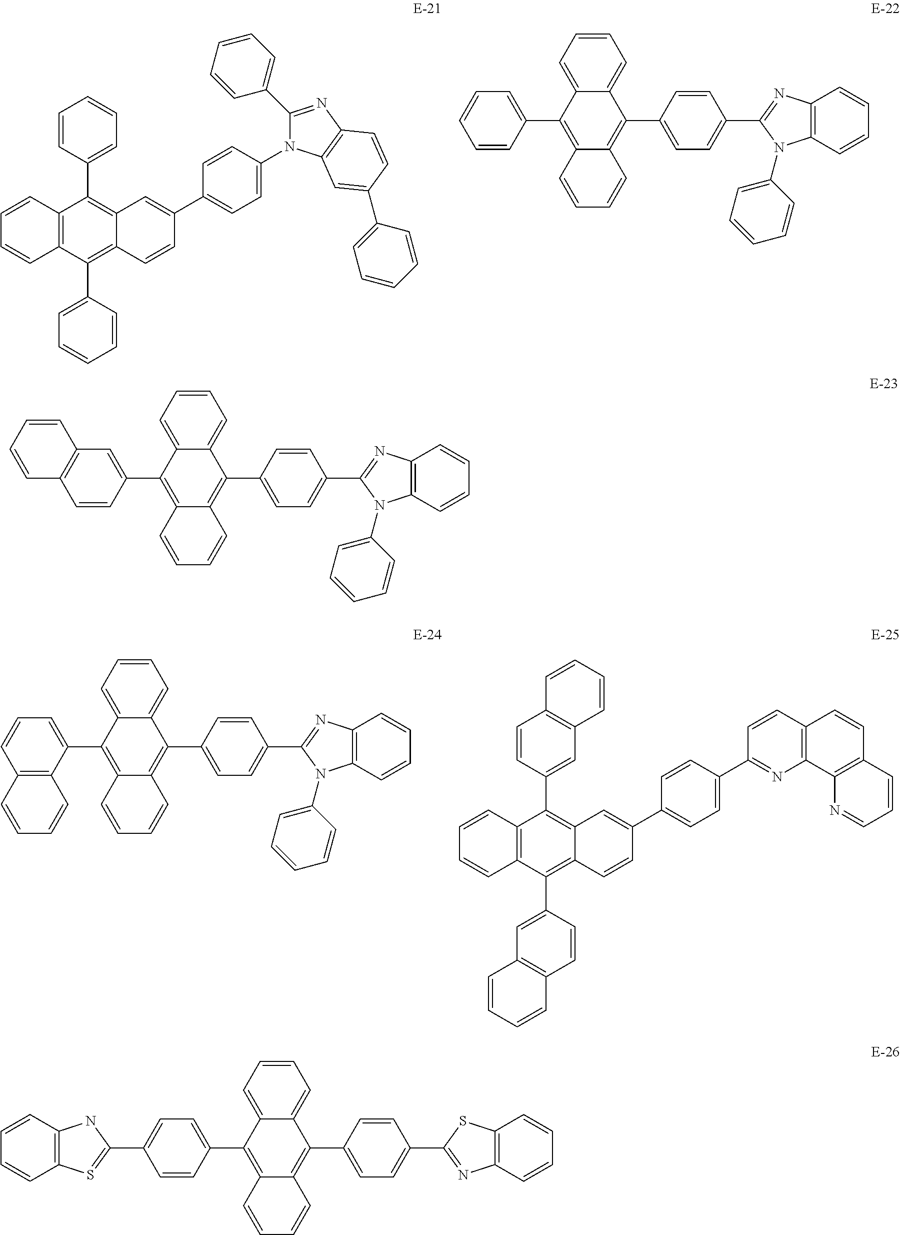

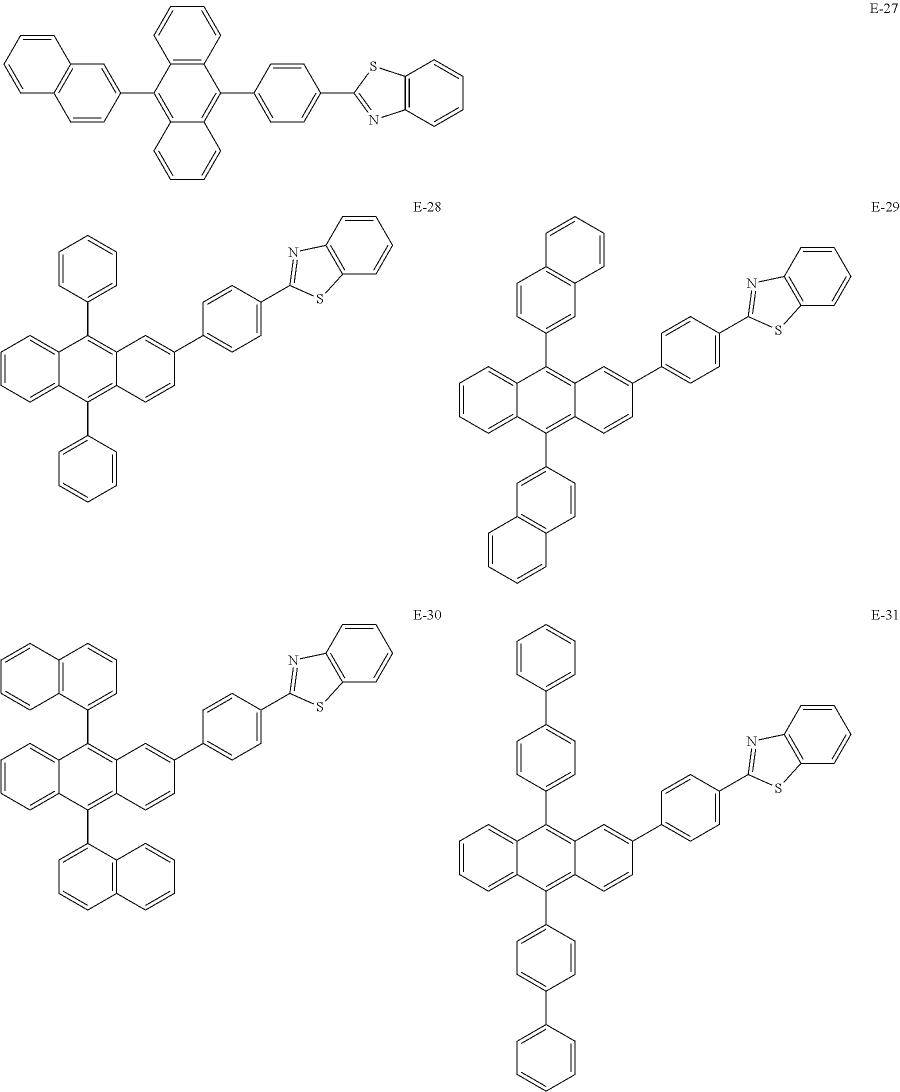 [SMILES: c1ccc2cc(-c3c4ccccc4c(-c4ccc(-c5nc6ccccc6s5)cc4)c4ccccc34)ccc2c1 ; c1ccc(-c2c3ccccc3c(-c3ccccc3)c3cc(-c4ccc(-c5nc6ccccc6s5)cc4)ccc23)cc1 ; c1ccc2cc(-c3c4ccccc4c(-c4ccc5ccccc5c4)c4cc(-c5ccc(-c6nc7ccccc7s6)cc5)ccc34)ccc2c1 ; c1ccc2c(-c3c4ccccc4c(-c4cccc5ccccc45)c4cc(-c5ccc(-c6nc7ccccc7s6)cc5)ccc34)cccc2c1 ; c1ccc(-c2ccc(-c3c4ccccc4c(-c4ccc(-c5ccccc5)cc4)c4cc(-c5ccc(-c6nc7ccccc7s6)cc5)ccc34)cc2)cc1]

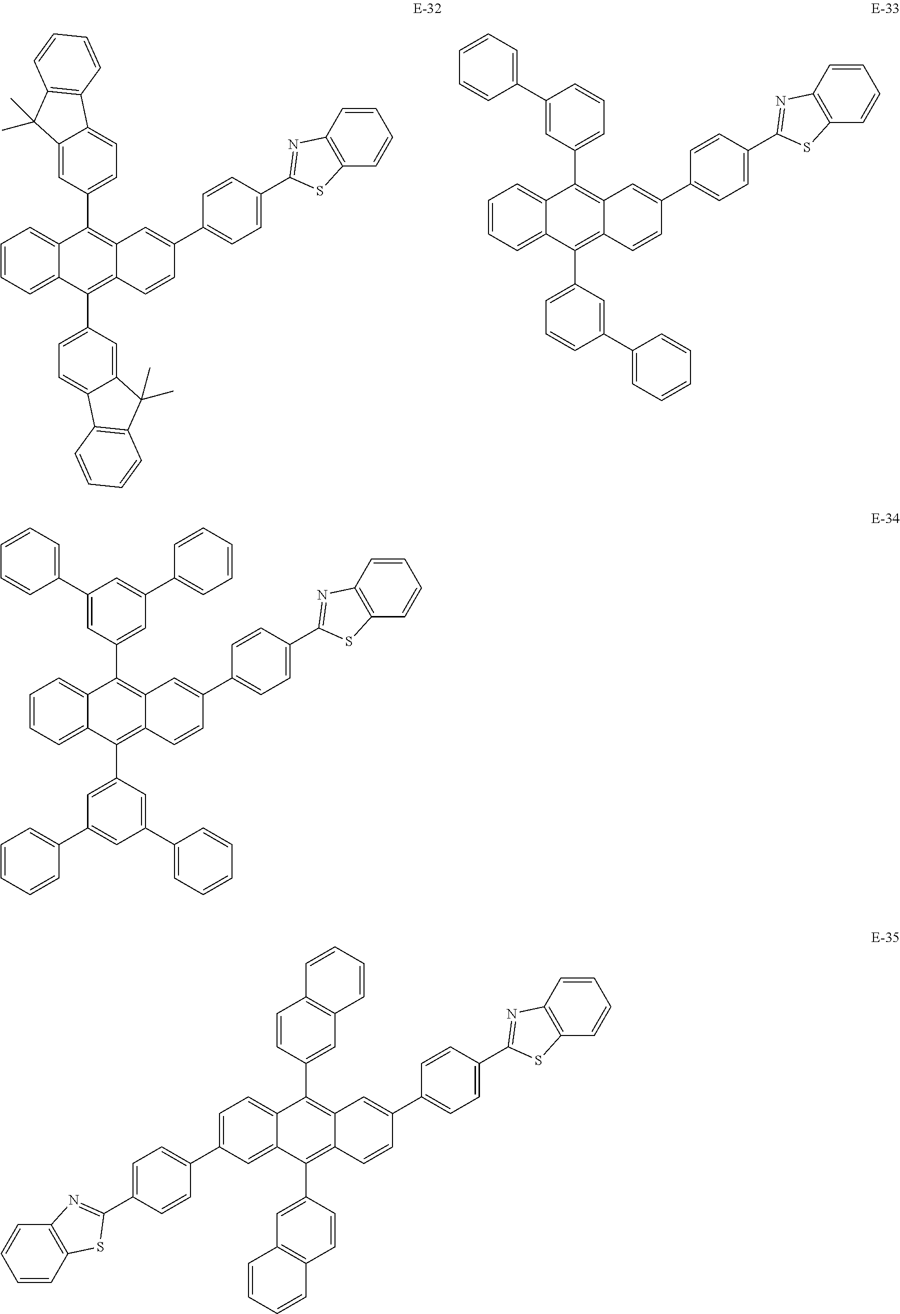 [SMILES: CC1(C)c2ccccc2-c2ccc(-c3c4ccccc4c(-c4ccc5c(c4)C(C)(C)c4ccccc4-5)c4cc(-c5ccc(-c6nc7ccccc7s6)cc5)ccc34)cc21 ; c1ccc(-c2cccc(-c3c4ccccc4c(-c4cccc(-c5ccccc5)c4)c4cc(-c5ccc(-c6nc7ccccc7s6)cc5)ccc34)c2)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7s6)cc5)ccc34)c2)cc1 ; c1ccc2cc(-c3c4ccc(-c5ccc(-c6nc7ccccc7s6)cc5)cc4c(-c4ccc5ccccc5c4)c4ccc(-c5ccc(-c6nc7ccccc7s6)cc5)cc34)ccc2c1]

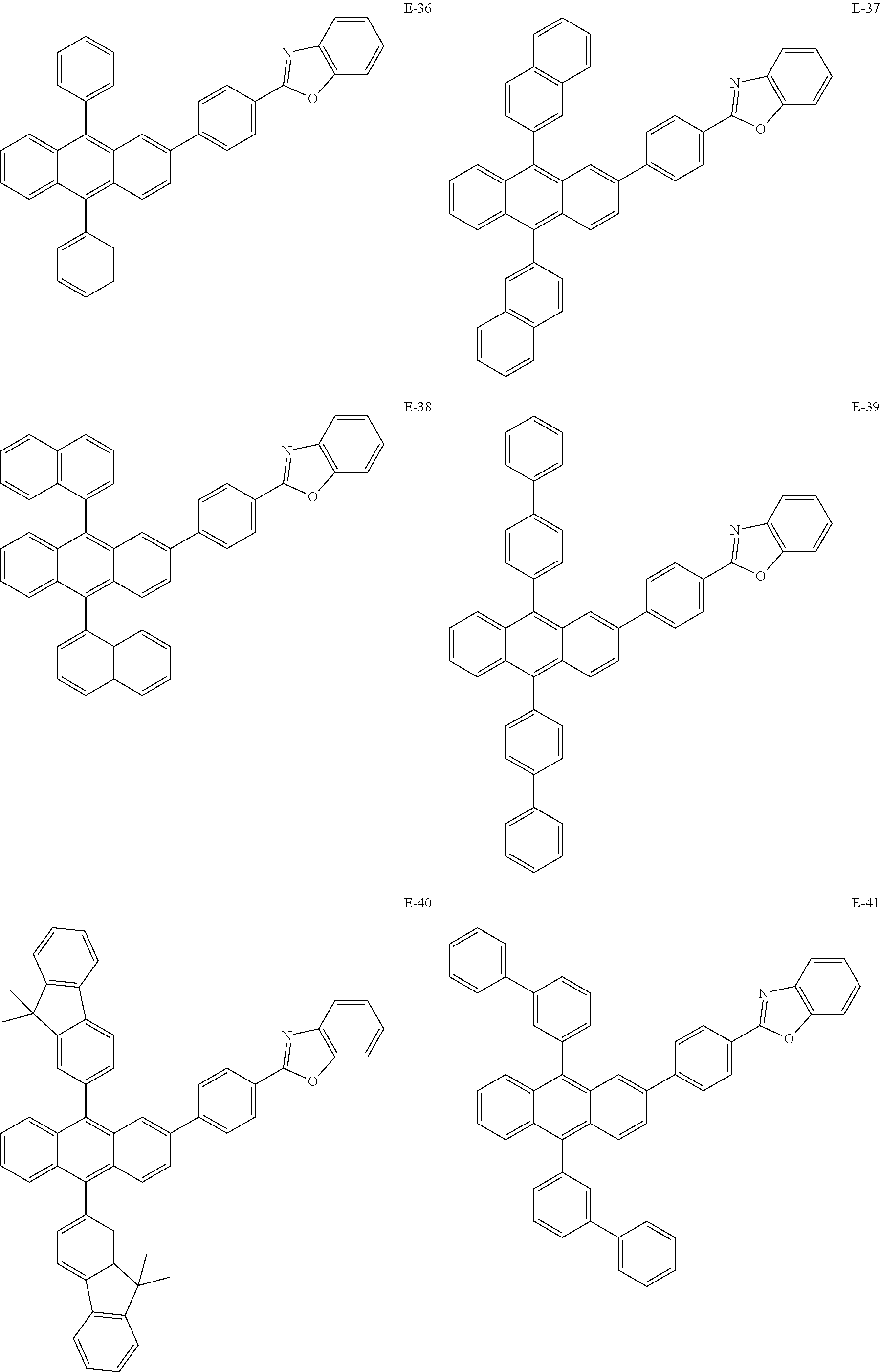

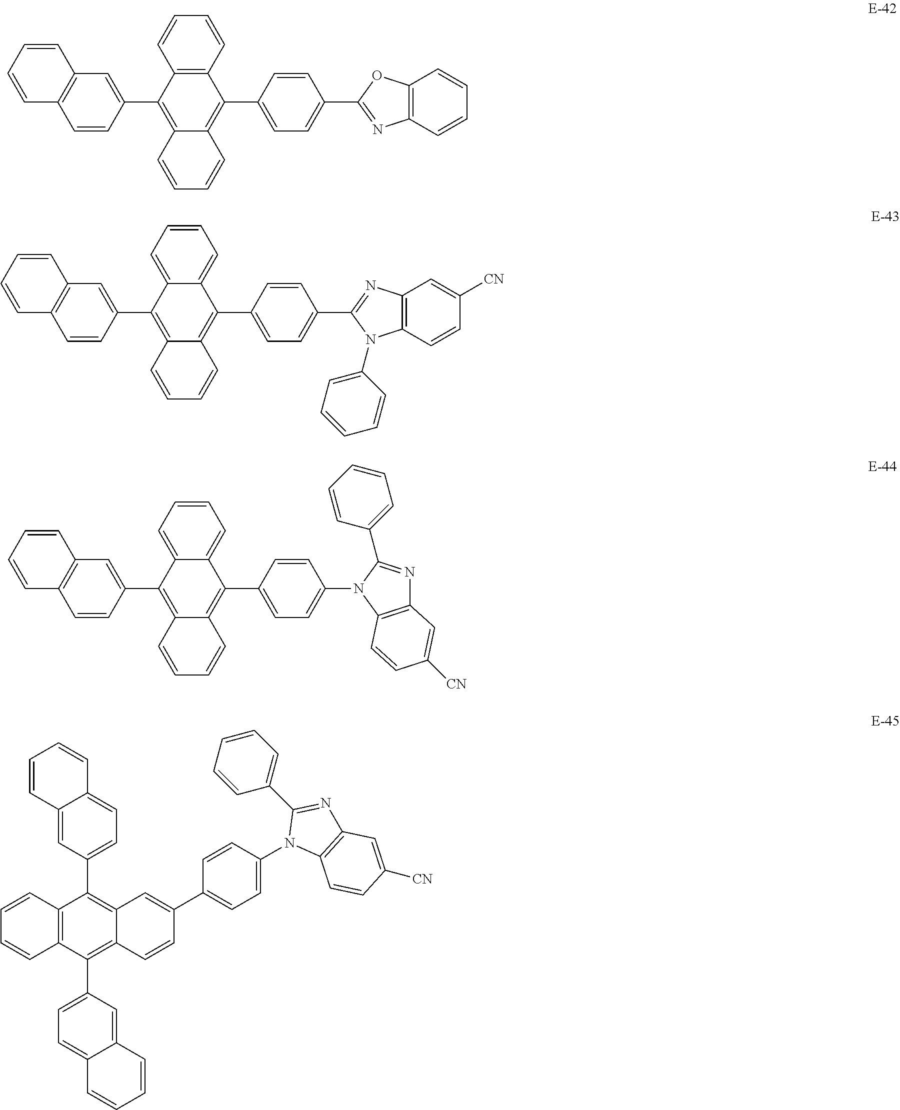

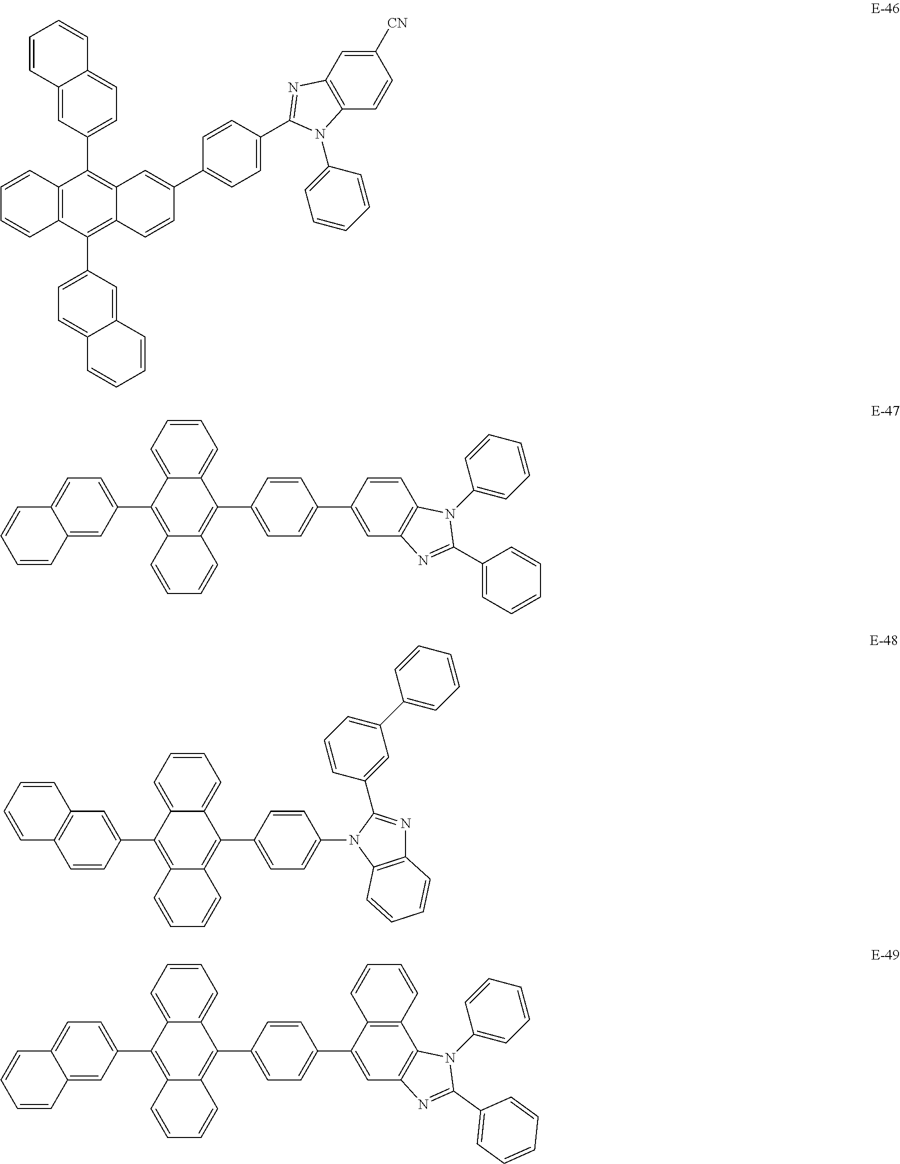

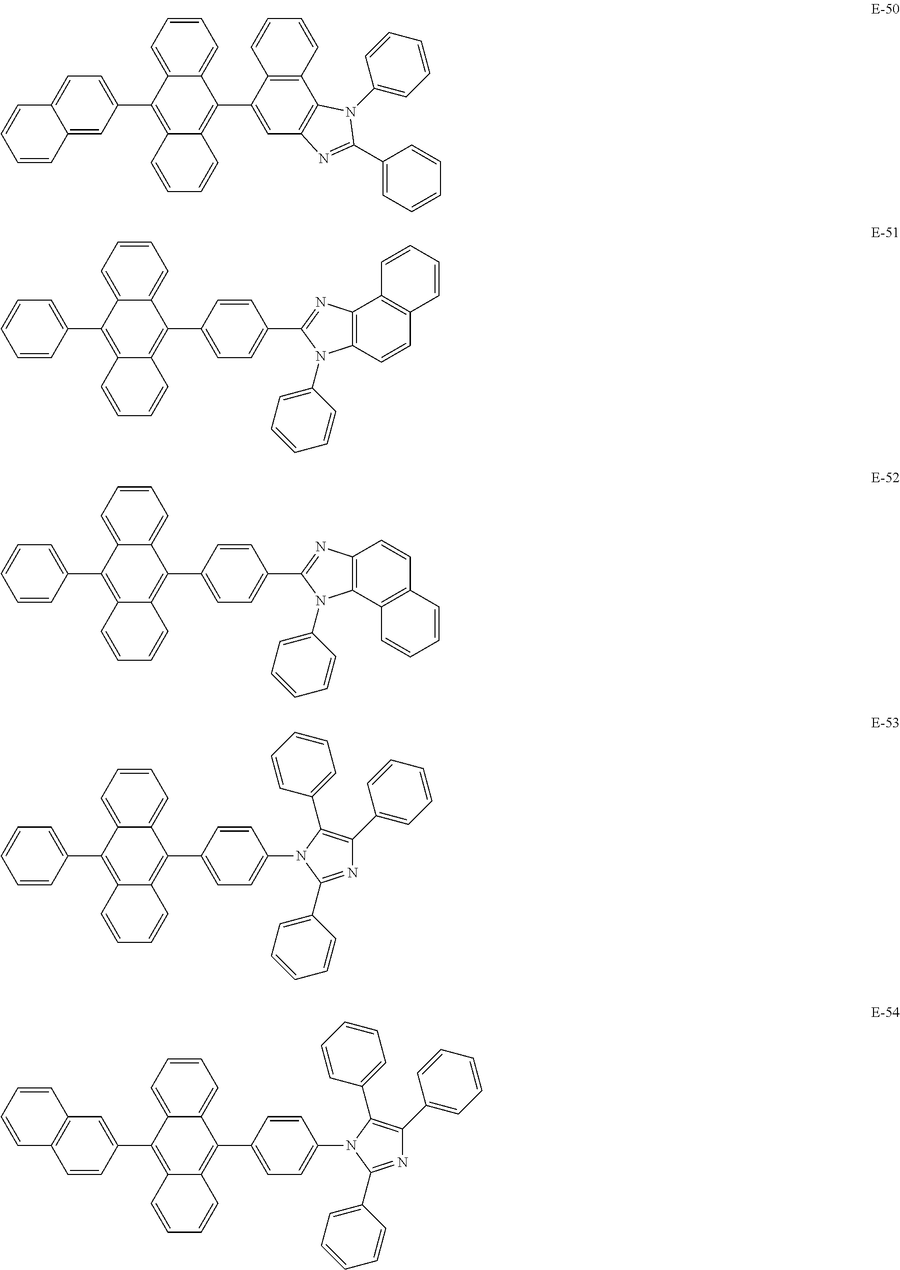

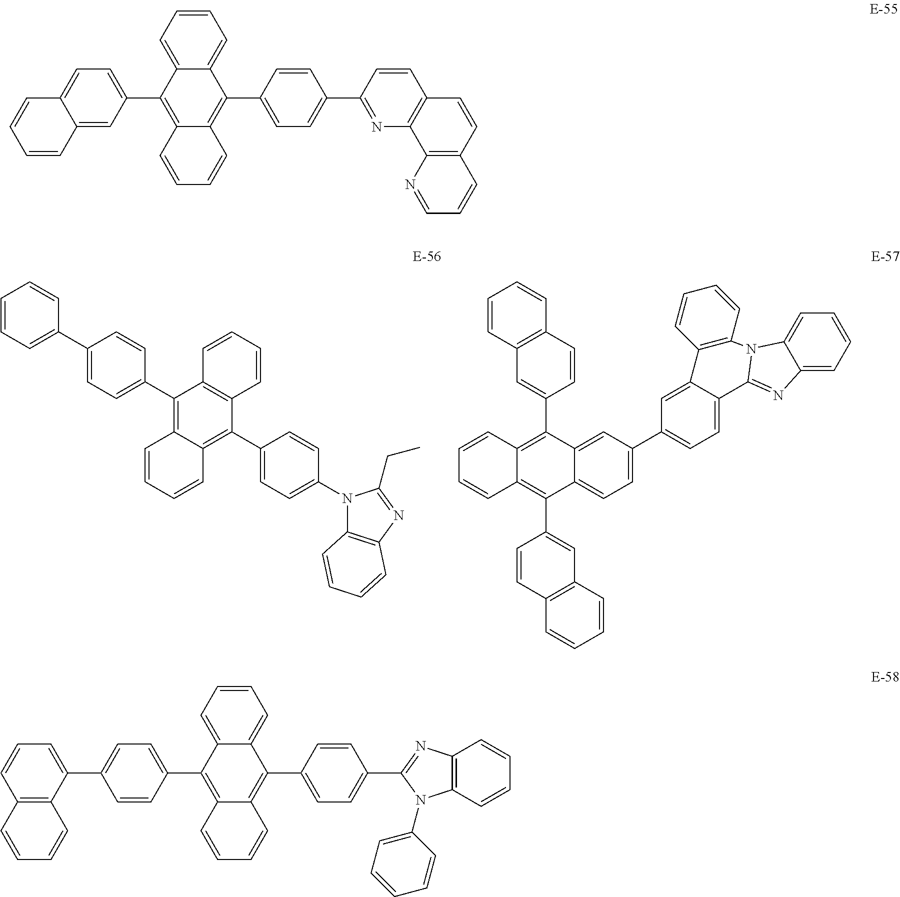

The reductive dopant may be at least one selected from the group consisting of alkali metals, alkali metal compounds, alkaline earth metals, rare-earth metals, and halides, oxides, and complexes thereof. Specifically, the reductive dopant includes lithium quinolate, sodium quinolate, cesium quinolate, potassium quinolate, LiF, NaCl, CsF, Li₂O, BaO, and BaF₂, but is not limited thereto.

The electron transport zone may comprise an electron transport layer, an electron injection layer, or both of them. The electron transport layer and the electron injection layer, each independently, may be composed of two or more layers. LUMO energy level of the electron buffering layer may be higher or lower than LUMO energy level of the electron transport zone. In the present disclosure, specifically, LUMO energy level of the electron transport zone may indicate the level of an electron transport material comprised in the electron transport zone. When the electron transport zone has two or more layers, LUMO energy level of the electron transport zone may be LUMO energy level of a material comprised in a layer which is in the electron transport zone and is adjacent to the electron buffering layer.

LUMO energy level can be easily measured by known various methods. Generally, cyclic voltammetry or ultraviolet photoelectron spectroscopy (UPS) may be used. Therefore, one skilled in the art can easily understand and determine the electron buffering layer, the host material, and the electron transport zone which satisfy the aforementioned relationship for LUMO energy levels, so that one can easily practice the invention. HOMO energy level can be easily measured in the same manner as LUMO energy level.

The layers of the organic electroluminescent device of the present disclosure can be formed in the order of the light-emitting layer, the electron buffering layer, the electron transport zone, and the second electrode, or in the order of the light-emitting layer, the electron transport zone, the electron buffering layer, and the second electrode.

The organic electroluminescent device of the present disclosure may further comprise a hole injection layer, a hole transport layer, or both between the first electrode and the light-emitting layer.

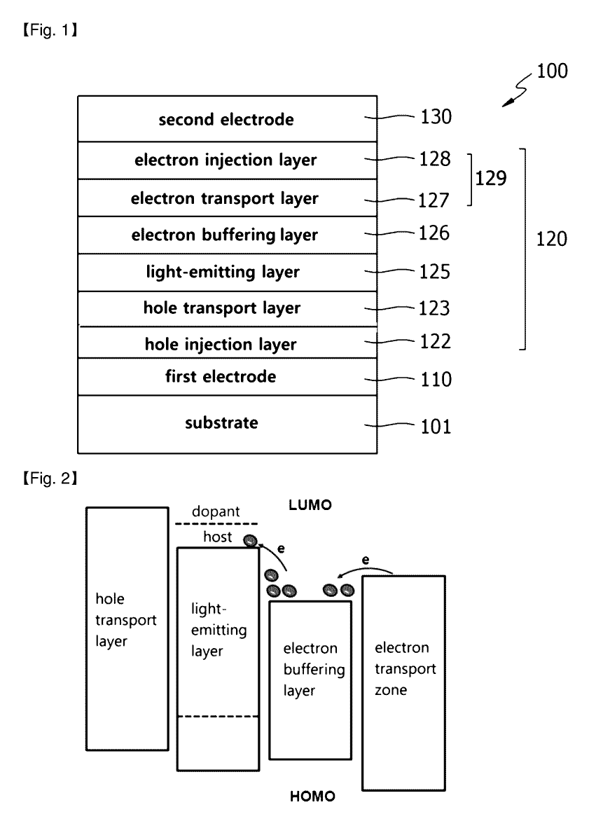

Hereinafter, referring to FIG. 1, the structure of an organic electroluminescent device, and a method for preparing it will be described in detail.

FIG. 1 shows an organic electroluminescent device 100 comprising a substrate 101, a first electrode 110 formed on the substrate 101, an organic layer 120 formed on the first electrode 110, and a second electrode 130 formed on the organic layer 120 and facing the first electrode 110.

The organic layer 120 comprises a hole injection layer 122, a hole transport layer 123 formed on the hole injection layer 122, a light-emitting layer 125 formed on the hole transport layer 123, an electron buffering layer 126 formed on the light-emitting layer 125, and an electron transport zone 129 formed on the electron buffering layer 126; and the electron transport zone 129 comprises an electron transport layer 127 formed on the electron buffering layer 126, and an electron injection layer 128 formed on the electron transport layer 127. The hole injection layer 122, the hole transport layer 123, the light-emitting layer 125, the electron buffering layer 126, the electron transport layer 127 and the electron injection layer 128 may be a single layer, or may be composed of two or more layers.

The substrate 101 may be any conventional substrate for an organic electroluminescent device, such as a glass substrate, a plastic substrate, or a metal substrate.

The first electrode 110 may be an anode, and may be prepared with a high work-function material.

The hole injection layer 122 may be prepared with any hole injection material known in the art, specifically a phthalocyanine compound such as copper phthalocyanine, MTDATA(4,4′,4″-tris[(3-methylphenyl)phenylamino]triphenylamine), 2-TNATA(4,4′,4″-tris[2-napthyl(phenyl)amino]triphenylamine), N1,N1′-([1,1′-biphenyl]-4,4′-diyl)bis(N1-(naphthalene-1-yl)-N4,N4-diphenylbenzene-1,4-diamine), Pani/DBSA (polyaniline/dodecylbenzenesulfonic acid), PEDOT/PSS(poly(3,4-ethylenedioxythiophene)/poly(4-styrenesulfonate)), Pani/CSA (polyaniline/camphor sulfonic acid), or Pani/PSS (polyaniline/poly(4-styrenesulfonate)), but is not limited thereto.

The hole injection layer 122 may be formed of a compound represented by the following formula 7:

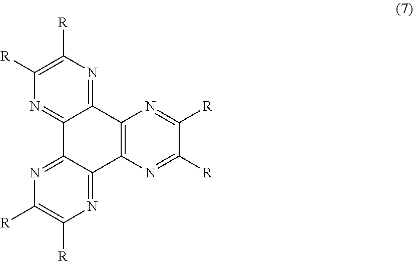

wherein R may be selected from the group consisting of a cyano (—CN), a nitro (—NO₂), a phenylsulfonyl(—SO₂(C₆H₅)), a cyano- or nitro-substituted (C2-C5) alkenyl, and a cyano- or nitro-substituted phenyl.

The compound of formula 7 has a characteristic to be crystallized. Thus, by using the compound, the hole injection layer 122 can have strength. The example of the compound of formula 7 includes HAT-CN (1,4,5,8,9,12-hexaazatriphenylene-hexacarbonitrile).

The hole transport layer 123 may be prepared with any hole transport material known in the art, specifically aromatic amine-based derivatives, especially biphenyldiamine-based derivatives such as TPD(N,N′-bis-(3-methylphenyl)-N,N′-diphenylbenzidine), N4,N4,N4′,N4′-tetra([1,1′-biphenyl]-4-yl)-[1,1′-biphenyl]-4,4′-diamine, and the compound represented by the following formulae, but is not limited thereto.

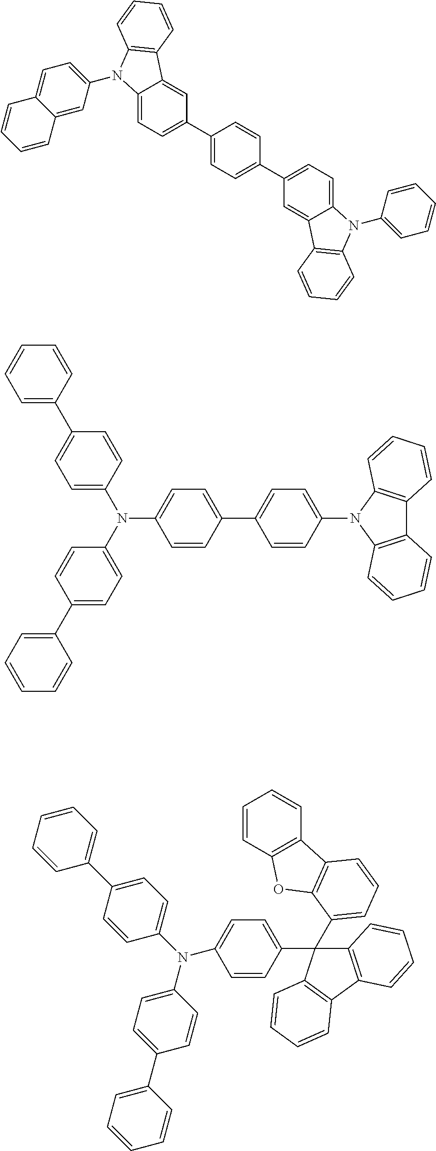 [SMILES: c1ccc(-n2c3ccccc3c3cc(-c4ccc(-c5ccc6c(c5)c5ccccc5n6-c5ccc6ccccc6c5)cc4)ccc32)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(C4(c5cccc6c5oc5ccccc56)c5ccccc5-c5ccccc54)cc3)cc2)cc1]

The light-emitting layer 125 may comprise a host compound and a dopant compound, which are not particularly limited, and may be preferably selected from the known compounds. Examples of the host compound and the dopant compound are as previously described in detail. When the light-emitting layer 125 comprises a host and a dopant, the dopant can be doped in an amount of less than about 25 wt %, and preferably less than 17 wt %, based on the total amount of the dopant and host of the light-emitting layer. When the light emitting layer 125 is composed of two or more layers, each of the layers may be prepared to emit color different from one another. For example, the device may emit white light by preparing three light-emitting layers 125 which emit blue, red, and green, respectively.

The electron buffering layer 126 employs the compound of formula 1 of the present disclosure. The details of the compound of formula 1 are as previously described. The thickness of the electron buffering layer 126 is 1 nm or more, but is not particularly limited thereto. Specifically, the thickness of the electron buffering layer 126 may be in the range of from 2 nm to 200 nm. The electron buffering layer 126 may be formed on the light-emitting layer 125 by using various known methods such as vacuum deposition, wet film-forming methods, laser induced thermal imaging, etc.

The electron transport layer 127 may be prepared with any electron transport material known in the art. Examples of the electron transport material are as previously described in detail. Preferably, the electron transport layer 127 may be a mixed layer comprising an electron transport compound and a reductive dopant. In this case, the electron transport compound is reduced to an anion, and thus it becomes easier to inject and transport electrons to an electroluminescent medium. Examples of the reductive dopant are as previously described in detail.

The electron injection layer 128 may be prepared with any electron injection material known in the art, which includes lithium quinolate, sodium quinolate, cesium quinolate, potassium quinolate, LiF, NaCl, CsF, Li₂O, BaO, and BaF₂, but is not limited thereto.

The second electrode 130 may be a cathode, and may be prepared with a low work-function material.

The aforementioned description regarding the organic electroluminescent device shown in FIG. 1 is intended to explain one embodiment of the invention, and is not meant in any way to restrict the scope of the invention. The organic electroluminescent device can be constructed in another way. For example, any one optional component such as a hole injection layer may not be comprised in the organic electroluminescent device of FIG. 1, except for a light-emitting layer and an electron buffering layer. In addition, an optional component may be further comprised therein, which includes an impurity layer such as n-doping layer and p-doping layer. The organic electroluminescent device may be a both sides emission type in which a light-emitting layer is placed on each of both sides of the impurity layer. The two light-emitting layers on the impurity layer may emit different colors. The organic electroluminescent device may be a bottom emission type in which a first electrode is a transparent electrode and a second electrode is a reflective electrode. The organic electroluminescent device may be a top emission type in which a first electrode is a reflective electrode and a second electrode is a transparent electrode. The organic electroluminescent device may have an inverted type structure in which a cathode, an electron transport layer, a light-emitting layer, a hole transport layer, a hole injection layer, and an anode are sequentially stacked on a substrate.

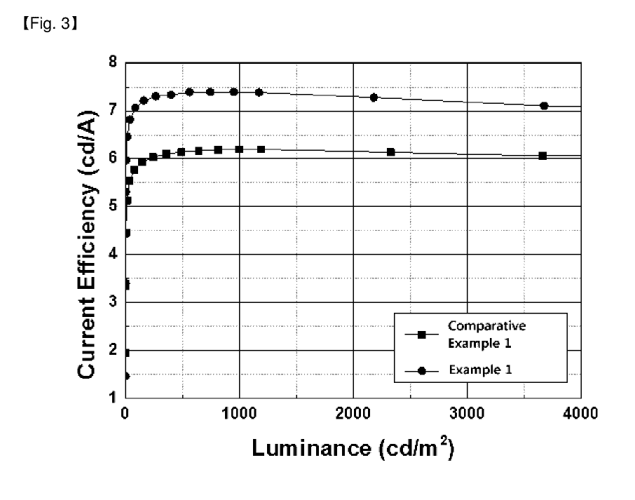

FIG. 2 is a schematic sectional view of an energy band diagram among a hole transport layer, a light-emitting layer, an electron buffering layer, and an electron transport zone of an organic electroluminescent device according to one embodiment of the present disclosure.

In FIG. 2, a hole transport layer 123, a light-emitting layer 125, an electron buffering layer 126, and an electron transport zone 129 are sequentially stacked. Electrons (e) injected from a cathode are transported to a light-emitting layer through an electron transport zone 129 and an electron buffering layer 126.

LUMO energy level of the electron buffering layer 126 may be similar to LUMO energy level of the light-emitting layer 125, and may be lower than LUMO energy level of an electron transport zone 129. Specifically, LUMO energy levels may have the following relationship: the electron transport zone>the electron buffering layer≈the host compound. The difference of LUMO energy level between the electron buffering layer and the host compound is not significant. However, the LUMO energy level of the electron buffering layer may be about ±0.2 eV, preferably about ±0.1 eV based on LUMO energy level of the host compound depending on the substitution position.

Hereinafter, a preparation method of an organic electroluminescent device according to one embodiment using the electron buffering material of the present disclosure, and luminescent properties of the device will be explained in detail with reference to the following examples.

### [Comparative Example 1] Preparation of a Blue-Emitting OLED in which an Electron Buffering Layer is not Comprised

OLED was produced as follows. A transparent electrode indium tin oxide (ITO) thin film (15 Ω/sq) on a glass substrate for an OLED (Geomatec) was subjected to an ultrasonic washing with trichloroethylene, acetone, ethanol, and distilled water, sequentially, and then was stored in isopropanol. The ITO substrate was then mounted on a substrate holder of a vacuum vapor depositing apparatus. N4,N4′-diphenyl-N4,N4′-bis(9-phenyl-9H-carbazol-3-yl)-[1,1′-biphenyl]-4,4′-diamine (HI-1) was introduced into a cell of the vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 60 nm on the ITO substrate. 1,4,5,8,9,12-hexaazatriphenylene-hexacarbonitrile (HAT-CN) (HI-2) was then introduced into another cell of the vacuum vapor depositing apparatus, and was evaporated by applying an electric current to the cell, thereby forming a second hole injection layer having a thickness of 5 nm on the first hole injection layer. N-([1,1′-biphenyl]-4-yl)-9,9-dimethyl-N-(4-(9-phenyl-9H-carbazol-3-yl)phenyl)-9H-fluoren-2-amine (HT-1) was then introduced into another cell of the vacuum vapor depositing apparatus, and was evaporated by applying an electric current to the cell, thereby forming a first hole transport layer having a thickness of 20 nm on the second hole injection layer. Thereafter, N,N-di([1,1′-biphenyl]-4-yl)-4′-(9H-carbazol-9-yl)-[1,1′-biphenyl]-4-amine (HT-2) was introduced into another cell of the vacuum vapor depositing apparatus, and was evaporated by applying an electric current to the cell, thereby forming a second hole transport layer having a thickness of 5 nm on the first hole transport layer. After forming the hole injection layer and the hole transport layer, a light-emitting layer was formed thereon as follows: compound B-10 was introduced into one cell of the vacuum vapor depositing apparatus, as a host material, and compound D-38 was introduced into another cell as a dopant. The two materials were evaporated at different rates, so that the dopant was deposited in a doping amount of 2 wt % based on the total amount of the host and dopant to form a light-emitting layer having a thickness of 20 nm on the hole transport layer. 2-(4-(9,10-di(naphthalene-2-yl)anthracene-2-yl)phenyl)-1-phenyl-1H-benzo[d]imidazole (ETL-1) was then introduced into one cell, and lithium quinolate was introduced into another cell. The two materials were evaporated at the same rate, so that they were respectively deposited in a doping amount of 50 wt % to form an electron transport layer having a thickness of 36 nm on the light-emitting layer. After depositing lithium quinolate (EIL-1) as an electron injection layer having a thickness of 2 nm on the electron transport layer, an Al cathode having a thickness of 80 nm was then deposited by another vacuum vapor deposition apparatus on the electron injection layer. Thus, an OLED was produced. All the material used for producing the OLED device were those purified by vacuum sublimation at 10⁻⁶ torr.

The driving voltage, luminous efficiency, and CIE color coordinate of the prepared organic electroluminescent device at 1,000 nit of luminance are shown in Table 1 below.

### [Examples 1 to 4] Preparation of a Blue-Emitting OLED According to the Present Disclosure

OLEDs were produced and evaluated in the same manner as in Comparative Example 1, except that a thickness of an electron transport layer was 27 nm, and an electron buffering layer having a thickness of 9 nm was interposed between a light-emitting layer and an electron transport layer. Electron buffering materials used in Examples 1 to 4 are shown in Table 1 below.

FIG. 3 shows a graph illustrating a current efficiency versus a luminance of the organic electroluminescent device prepared in Comparative Example 1 and Example 1. In addition, evaluation results of the devices prepared in Examples 1 to 4 were shown in Table 1 below.

TABLE 1

| | Electron | | Current | Color | Color | | |
|---|---|---|---|---|---|---|---|
| | Buffering | Voltage | Efficiency | Coordinate | Coordinate | LUMO | HOMO |
| | Material | (V) | (cd/A) | (x) | (y) | (eV) | (eV) |
| Comparative | — | 4.4 | 6.1 | 0.140 | 0.100 | | |
| Ex. 1 | | | | | | | |
| Example 1 | B-1 | 4.5 | 7.4 | 0.139 | 0.100 | 1.64 | 5.12 |
| Example 2 | B-2 | 4.3 | 7.3 | 0.139 | 0.099 | 1.76 | 5.23 |
| Example 3 | B-3 | 4.5 | 7.3 | 0.139 | 0.100 | 1.61 | 5.11 |
| Example 4 | B-4 | 4.6 | 6.7 | 0.139 | 0.101 | 1.58 | 5.06 |

### [Comparative Example 2] Preparation of a Blue-Emitting OLED in which an Electron Buffering Layer is not Comprised

OLEDs were produced and evaluated in the same manner as in Comparative Example 1, except that a thickness of an electron transport layer was 35 nm. The evaluation results of the devices prepared in Comparative Example 2 were shown in Table 2 below.

### [Comparative Examples 3 and 4] Preparation of a Blue-Emitting OLED Comprising a Conventional Electron Buffering Material

OLEDs were produced and evaluated in the same manner as in Example 1, except that BF-1 and BF-2 were used for an electron buffering material. Evaluation results of the devices prepared in Comparative Examples 3 and 4 were shown in Table 2 below.

### [Examples 5 to 11] Preparation of a Blue-Emitting OLED According to the Present Disclosure

OLEDs were produced and evaluated in the same manner as in Comparative Example 2, except that a thickness of an electron transport layer was 25 nm, and an electron buffering layer having a thickness of 5 nm was interposed between a light-emitting layer and an electron transport layer. Evaluation results of the devices and the electron buffering materials used in Examples 5 to 11 were shown in Table 2 below.

TABLE 2

| | Electron | | Current | Color | Color | | |
|---|---|---|---|---|---|---|---|
| | Buffering | Voltage | Efficiency | Coordinate | Coordinate | LUMO | HOMO |
| | Material | (V) | (cd/A) | (x) | (y) | (eV) | (eV) |
| Comparative | — | 4.3 | 5.9 | 0.140 | 0.099 | | |
| Ex. 2 | | | | | | | |
| Comparative | BF-1 | 5.5 | 4.1 | 0.141 | 0.109 | 1.62 | 4.98 |
| Ex. 3 | | | | | | | |
| Comparative | BF-2 | 5.0 | 5.7 | 0.140 | 0.106 | 1.50 | 5.29 |
| Ex. 4 | | | | | | | |
| Example 5 | B-5 | 4.3 | 6.6 | 0.139 | 0.098 | 1.63 | 5.13 |
| Example 6 | B-6 | 4.1 | 6.7 | 0.139 | 0.098 | 1.75 | 5.20 |
| Example 7 | B-7 | 4.4 | 6.4 | 0.139 | 0.100 | 1.63 | 5.13 |
| Example 8 | B-8 | 4.3 | 6.9 | 0.139 | 0.100 | 1.65 | 5.13 |
| Example 9 | B-9 | 4.2 | 6.5 | 0.140 | 0.103 | 1.74 | 5.06 |
| Example 10 | B-10 | 4.5 | 6.4 | 0.140 | 0.096 | 1.62 | 5.12 |
| Example 11 | B-11 | 4.5 | 6.3 | 0.140 | 0.095 | 1.64 | 5.13 |

It is recognized that due to rapid of electron current by the electron buffering material of the present disclosure, the devices of Examples 1 to 11 showed higher efficiencies than those of Comparative Examples 1 and 2 in which an electron buffering material was not comprised. From FIG. 3, it is recognized that the organic electroluminescent devices of Example 1 show higher current efficiencies over the entire range of luminance than the organic electroluminescent device of Comparative Example 1.

In particular, LUMO energy levels of the anthracene electron buffering group were about 1.6 eV, which showed rapid of electron current despite a barrier between an electron buffering layer and an electron transport layer considering that LUMO energy level of the electron transport layer was 1.8 eV. It may be caused by the intermolecular stacking effect according to molecule arrangement of an electron buffering material, or the intermolecular interaction of interface between an electron buffering layer and a light-emitting layer since the host and the electron buffering material are anthracene derivatives.

The Examples relate to the blue light-emitting device, but the electron buffering material comprising the compound represented by formula 1 may be applied to green and red light-emitting devices.

Also, the device according to the Examples may be applied to a phosphorescent device. For example, a phosphorescent device needs high T₁ energy value at a host compound, a hole transport layer formed the interface with a light-emitting layer, and an electron buffering layer, but the electron buffering material used in the present disclosure has low T₁ energy value. Although the structure does not sufficiently bind to the phosphorescent excitons, the electron buffering material comprising the compound represented by formula 1 of the present disclosure has rapid electron current, thereby a light-emitting region may be formed in the light-emitting layer. Thus, it is possible to obtain excellent luminous efficiency without a change of color coordinates.

Hereinafter, the experiments for characteristic analysis were conducted in order to learn the electron current properties by the electron buffering material of the present disclosure, as follows.

### [Characteristic Analysis 1]

Electron Only Device (EOD) comprising a light-emitting layer was produced. The relative electron current properties of the device according to the present disclosure were compared to the device in which an electron buttering material is not comprised, and the device comprising a conventional electron buffering material. The structure of the device is as follows.

Barium, 2,9-dimethyl-4,7-diphenyl-1,10-phenanthroline (BCP) were introduced into a cell of the vacuum vapor depositing apparatus, and then an electric current was applied to the cell to evaporate the above introduced material, thereby forming a hole blocking layer (HBL) having a thickness of 10 nm on the ITO substrate. Thereafter, compound B-10 was introduced into one cell of the vacuum vapor depositing apparatus as a host, and compound D-38 was introduced into another cell as a dopant. The two materials were evaporated at different rates, so that the dopant was deposited in a doping amount of 2 wt % based on the total amount of the host and dopant to form a light-emitting layer having a thickness of 20 nm on the hole transport layer. 2-(4-(9,10-di(naphthalene-2-yl)anthracene-2-yl)phenyl)-1-phenyl-1H-benzo[d]imidazole (ETL-1) was then introduced into one cell, and lithium quinolate was introduced into another cell. The two materials were evaporated at the same rate, so that they were respectively deposited in a doping amount of 50 wt % to form an electron transport layer having a thickness of 35 nm on the light-emitting layer. After depositing lithium quinolate in an electron injection layer having a thickness of 2 nm on the electron transport layer, an Al cathode having a thickness of 80 nm was then deposited by another vacuum vapor deposition apparatus on the electron injection layer. Thus, an OLED was produced. All the material used for producing the OLED device were those purified by vacuum sublimation at 10⁻⁶ torr. When there is electron buffering material, OLEDs were produced in the same manner as described above, except that a thickness of an electron transport layer was 30 nm, and an electron buffering layer having a thickness of 5 nm was interposed between a light-emitting layer and an electron transport layer. The voltages in current density 10 mA/cm² and 100 mA/cm² were shown in Table 3 below.

TABLE 3

| Electron | | |
|---|---|---|
| Buffering | Voltage (V) | Voltage (V) |
| Material | (10 mA/cm²) | (100 mA/cm²) |
| — | 3.6 | 5.2 |
| BF-1 | 4.5 | 6.0 |
| B-5 | 3.8 | 5.4 |
| B-6 | 3.6 | 5.1 |
| B-9 | 3.7 | 5.3 |

It was confirmed that the electron current properties of the OLEDs comprising an electron buffering material containing anthracene derivatives B-5, B-6 and B-9 are similar to those of the OLEDs not comprising an electron buffering material. However, it was recognized that the electron buffering material comprising quinoline derivative BF-1 had lower driving voltage, although it had LUMO energy level similar to anthracene derivatives.

### [Characteristic Analysis 2]

The difference of the electron buffering materials of the present disclosure which comprise anthracene derivatives compared to Comparative Examples 3 and 4 was confirmed by comparing the dipole moment value calculated by Density Functional Theory (DFT). As a result, it was confirmed that the anthracene derivatives had a lower dipole moment value, which means the molecular array arranged in a flat state contributes to the improvement of the charge carrier injection properties (see Appl. Phys. Lett. 95, 243303 (2009), and Appl. Phys. Lett. 99, 123303 (2011)).

The dipole moment and the LUMO energy level according to an electron buffering material were shown in Table 4 below. BF-1 and B-3, BF-2 and B-4 have similar LUMO energy levels, respectively, but BF-1 and BF-2 are the electron buffering layer containing heterocyclic derivatives, while B-3 and B-4 are the electron buffering layer containing anthracene derivatives. Comparing BF-1 and B-3, BF-2 and B-4, respectively, B-3 and B-4 containing anthracene derivatives have excellent efficiency, although they have similar LUMO energy levels, which can be seen as a correlation of the dipole moment. It was recognized that anthracene derivatives B-3 and B-4 have a lower dipole moment value as the plate-shaped form, and have rapid electric current characteristics and excellent efficiency compared to heterocyclic derivatives.

TABLE 4

| Electron | Transition | |
|---|---|---|
| Buffering | Dipole Moment | LUMO |
| Material | (debye) | (eV) |
| BF-1 | 2.330 | 1.62 |
| BF-2 | 1.945 | 1.50 |
| B-3 | 0.492 | 1.61 |
| B-4 | 0.568 | 1.58 |

TABLE 5
Compounds in Comparative Examples and Examples

| | |
|---|---|
| A hole injection layer/ A hole transport layer | 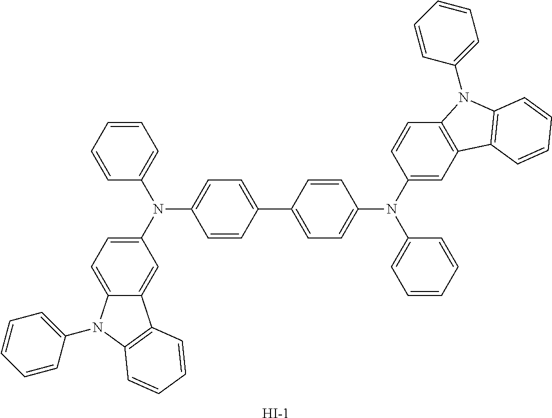 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] |
| | 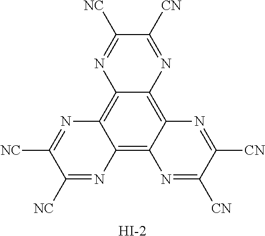 |
| | 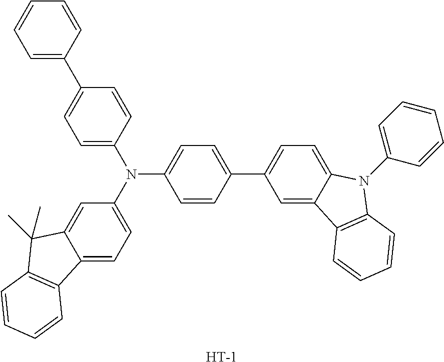 |
| | 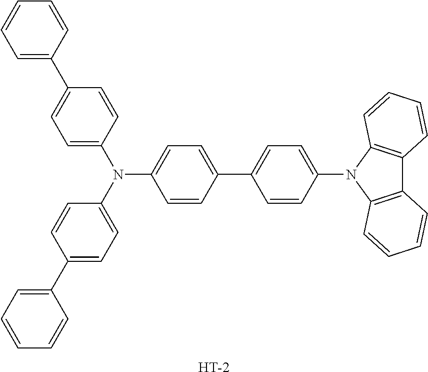 |
| A light-emitting layer | 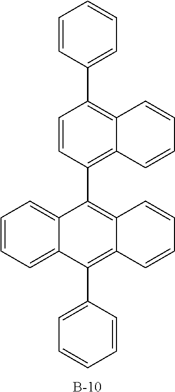 |
| | 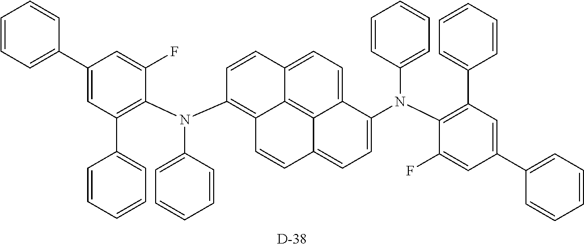 |
| An electron buffering layer | 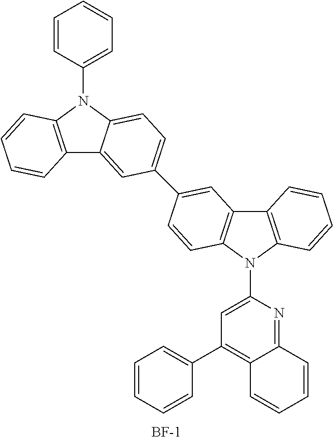 |
| | 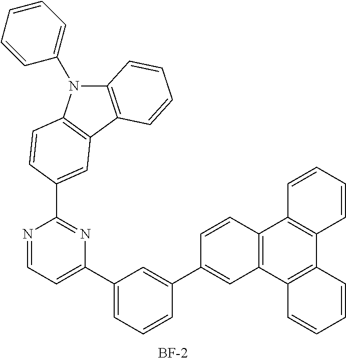 |
| An electron injection layer/ An electron transport layer | 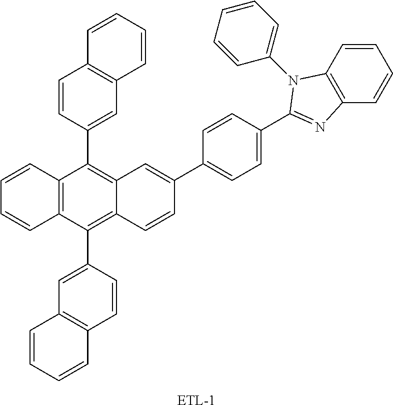 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; "ETL-1"] |
| | 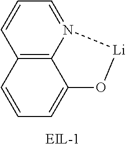 |
| A hole blocking layer | 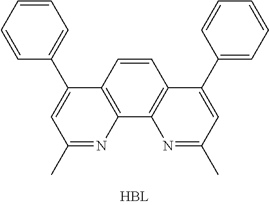 [SMILES: Cc1cc(-c2ccccc2)c2ccc3c(-c4ccccc4)cc(C)nc3c2n1] |

### DESCRIPTION OF REFERENCE NUMERALS

100: Organic electroluminescent device
101: Substrate
110: First electrode
120: Organic layer
122: Hole injection layer
123: Hole transport layer
125: Light-emitting layer
126: Electron buffering layer
127: Electron transport layer
128: Electron injection layer
129: Electron transport zone
130: Second electrode

1. An electron buffering material comprising a compound represented by the following formula 1: 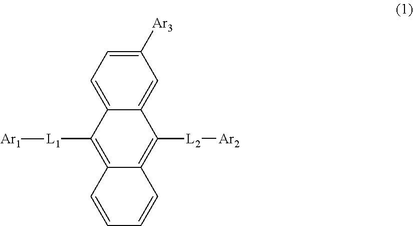
wherein
L₁ and L₂ each independently represent a single bond, a substituted or unsubstituted (C6-C30)arylene, or a substituted or unsubstituted (5- to 40-membered)heteroarylene;
Ar₁ to Ar₃ each independently represent hydrogen, a substituted or unsubstituted (C6-C30)aryl, or a substituted or unsubstituted (5- to 40-membered)heteroaryl;
the heteroaryl(ene) contains at least one hetero atom selected from B, N, O, S, P(═O), Si, and P.

2. The electron buffering material according to claim 1, wherein in formula 1, the substituents of the substituted (C6-C30)aryl(ene) and the substituted (5- to 40-membered)heteroaryl(ene) in L₁, L₂, and Ar₁ to Ar₃, each independently, are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C3-C30)cycloalkyl, a (3- to 7-membered)heterocycloalkyl, a (C6-C30)aryloxy, a (3- to 30-membered)heteroaryl unsubstituted or substituted with a (C6-C30)aryl, a (C6-C30)aryl unsubstituted or substituted with a (3- to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

3. The electron buffering material according to claim 1, wherein L₁ and L₂ each independently represent a single bond, or a substituted or unsubstituted (C6-C30)arylene; Ar₁ and Ar₂ each independently represent a substituted or unsubstituted (C6-C30)aryl, or a substituted or unsubstituted (5- to 35-membered)heteroaryl; and Arₐ represents hydrogen, or a substituted or unsubstituted (C6-C30)aryl.

4. The electron buffering material according to claim 1, wherein the compound represented by formula 1 is selected from the group consisting of:

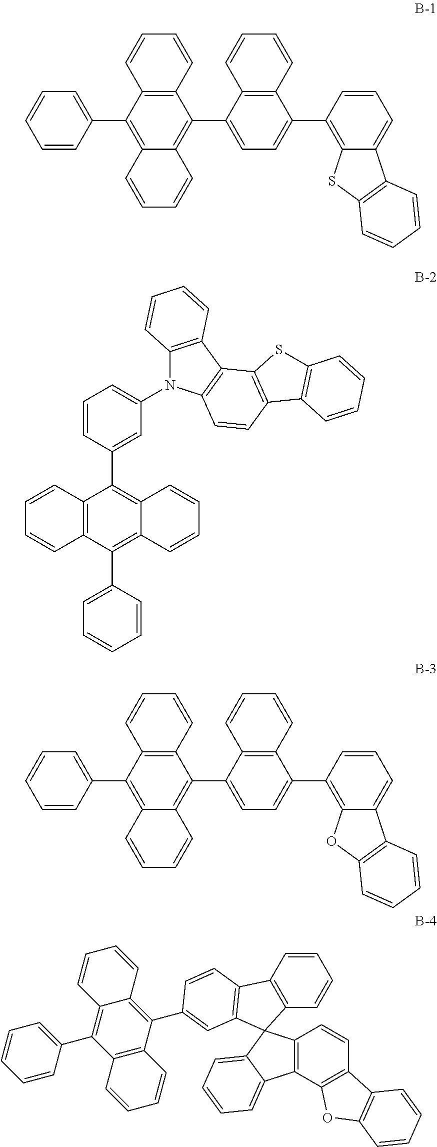 [SMILES: c1ccc(-c2c3ccccc3c(-c3ccc(-c4cccc5c4sc4ccccc45)c4ccccc34)c3ccccc23)cc1 ; c1ccc(-c2c3ccccc3c(-c3cccc(-n4c5ccccc5c5c6sc7ccccc7c6ccc54)c3)c3ccccc23)cc1 ; c1ccc(-c2c3ccccc3c(-c3ccc(-c4cccc5c4oc4ccccc45)c4ccccc34)c3ccccc23)cc1 ; c1ccc(-c2c3ccccc3c(-c3ccc4c(c3)C3(c5ccccc5-4)c4ccccc4-c4c3ccc3c4oc4ccccc43)c3ccccc23)cc1]

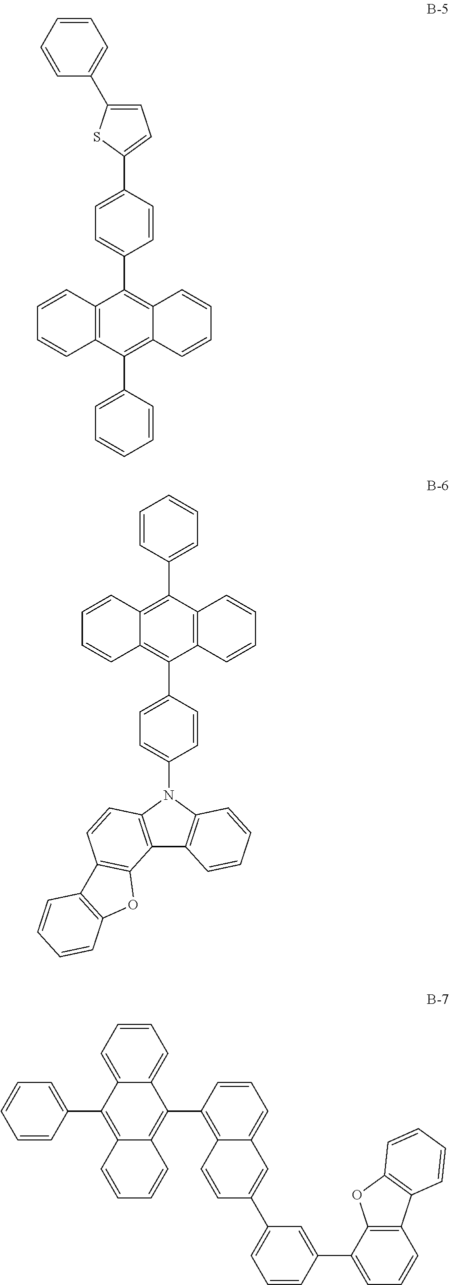 [SMILES: c1ccc(-c2ccc(-c3ccc(-c4c5ccccc5c(-c5ccccc5)c5ccccc45)cc3)s2)cc1 ; c1ccc(-c2c3ccccc3c(-c3ccc(-n4c5ccccc5c5c6oc7ccccc7c6ccc54)cc3)c3ccccc23)cc1 ; c1ccc(-c2c3ccccc3c(-c3cccc4cc(-c5cccc(-c6cccc7c6oc6ccccc67)c5)ccc34)c3ccccc23)cc1]

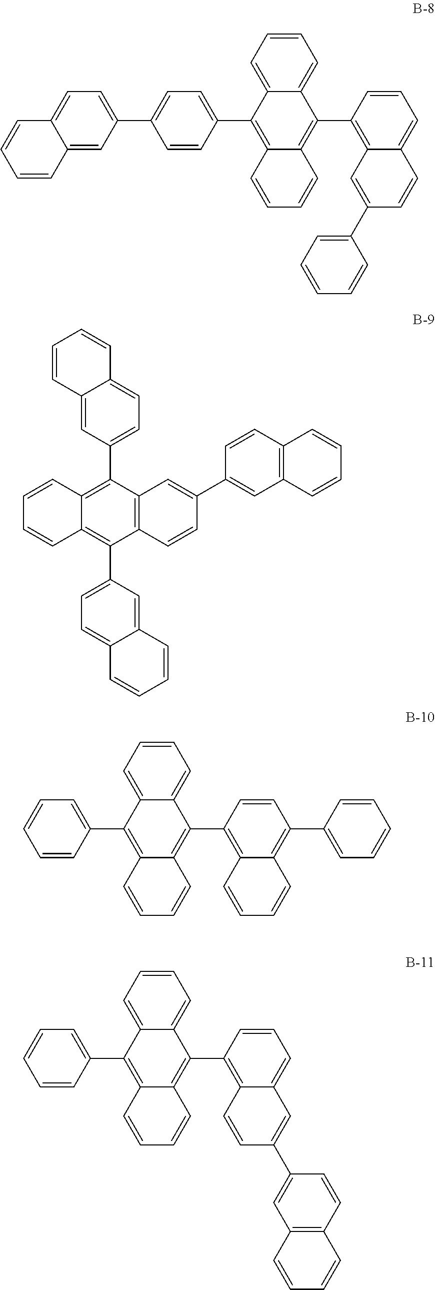

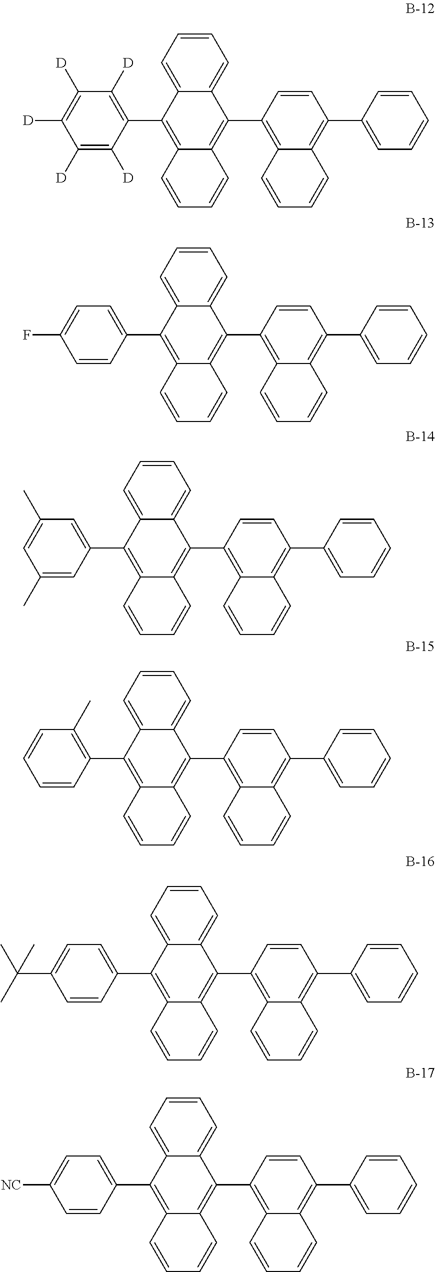

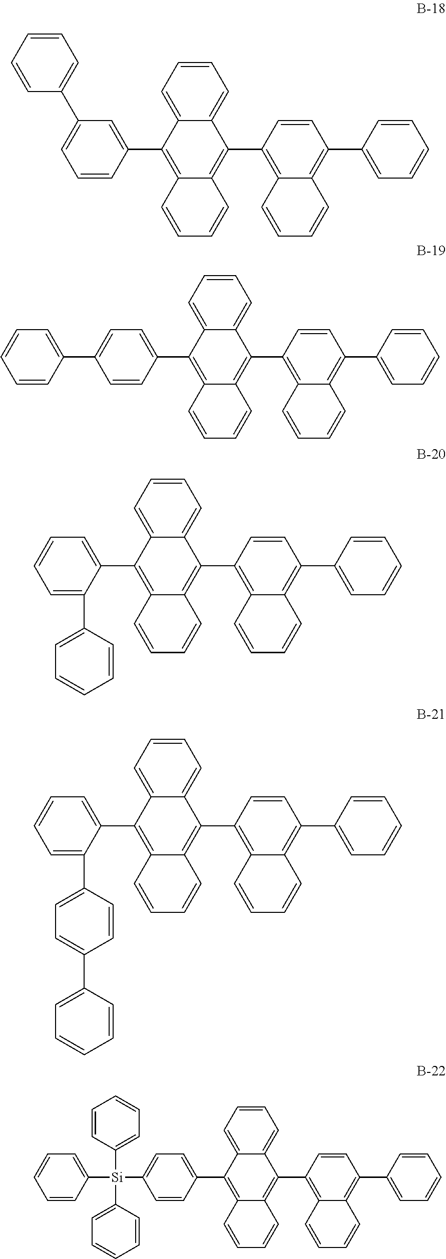

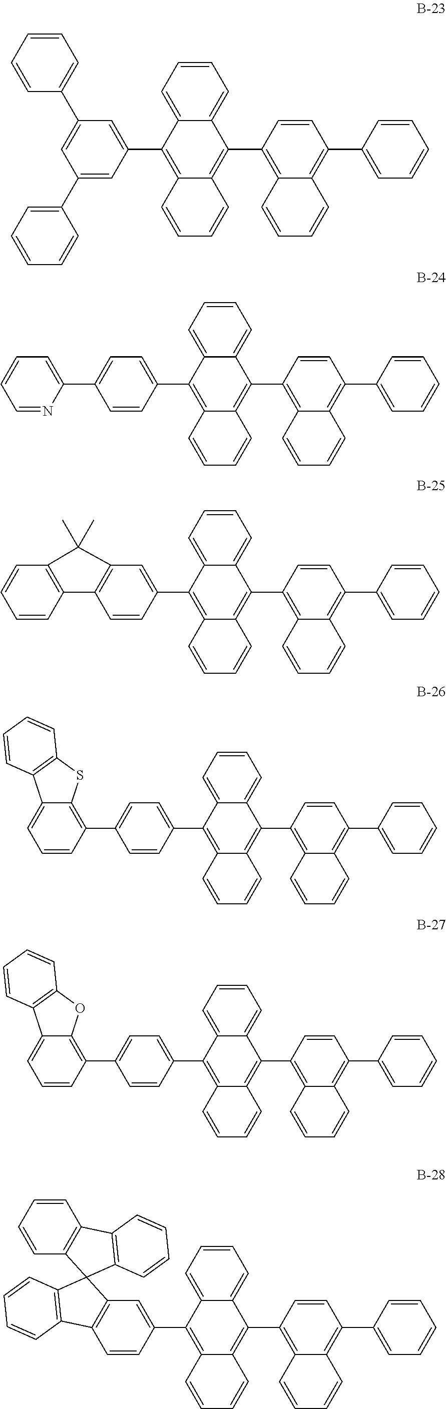

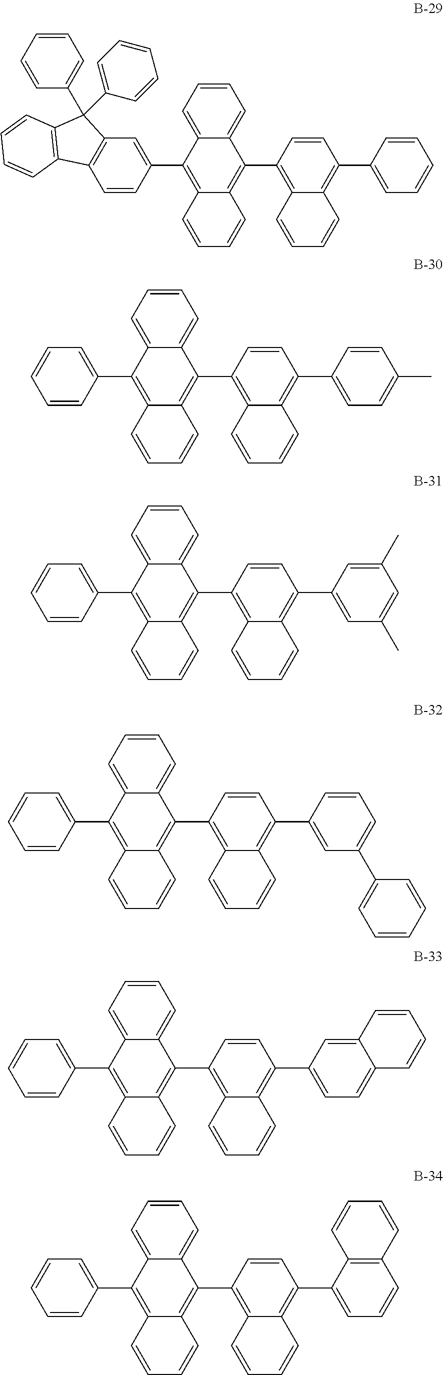

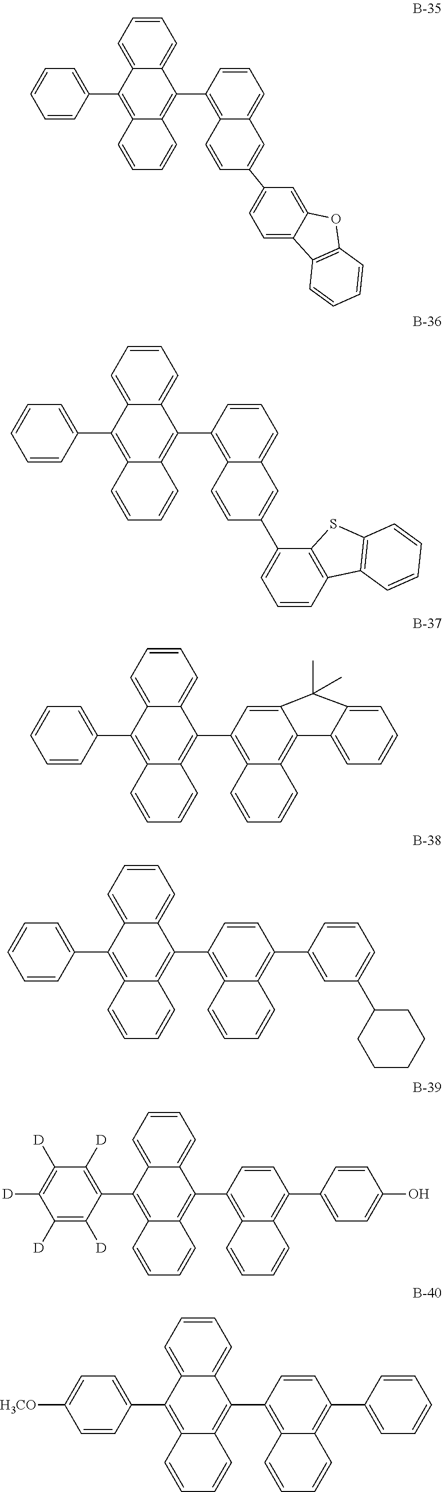

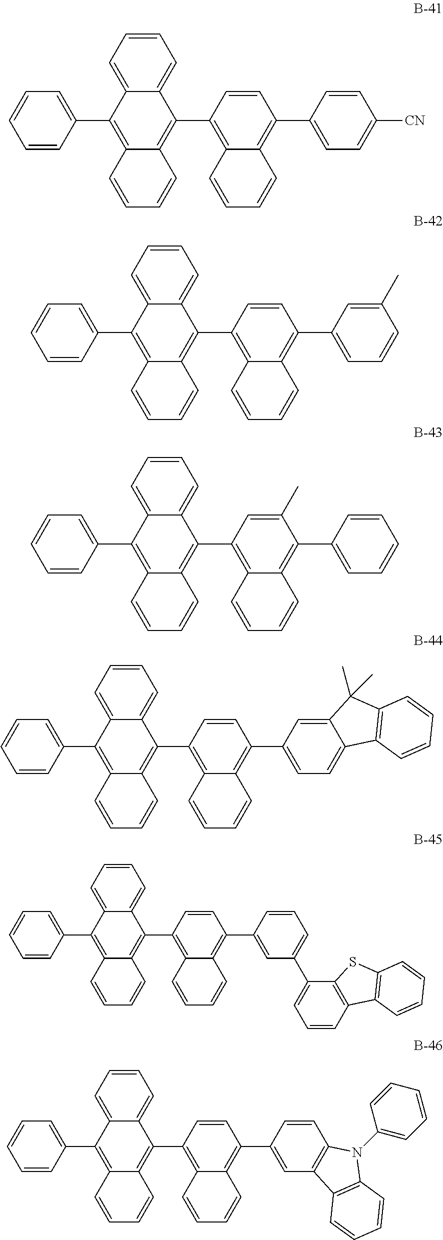

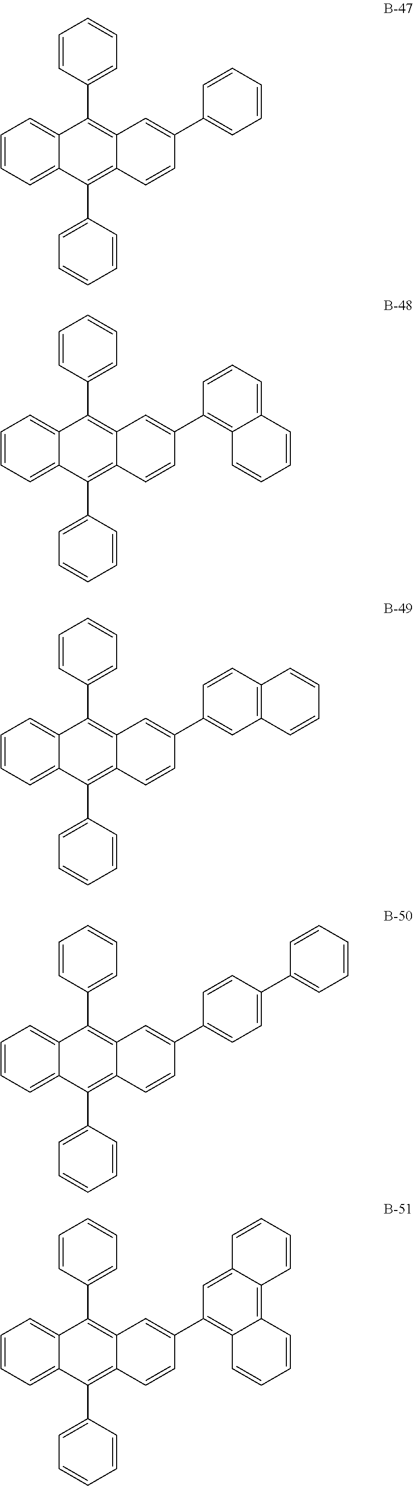

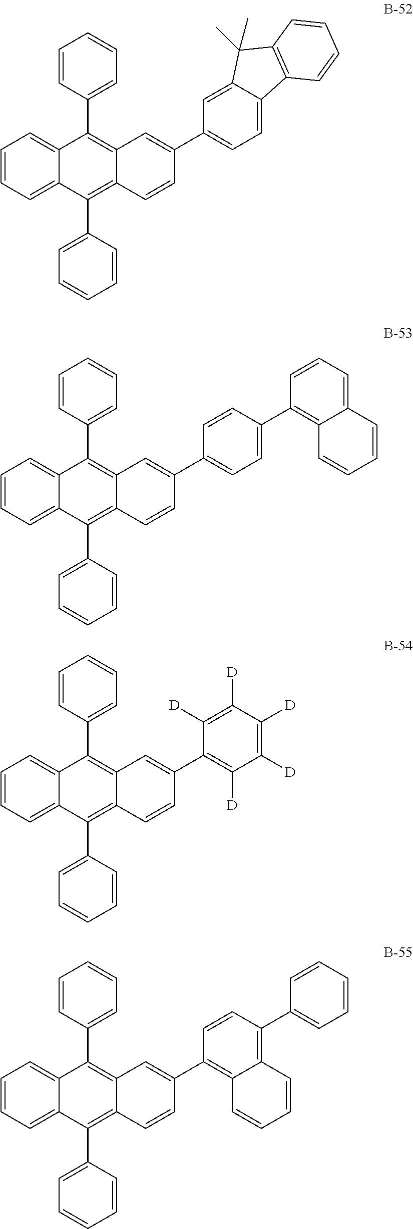

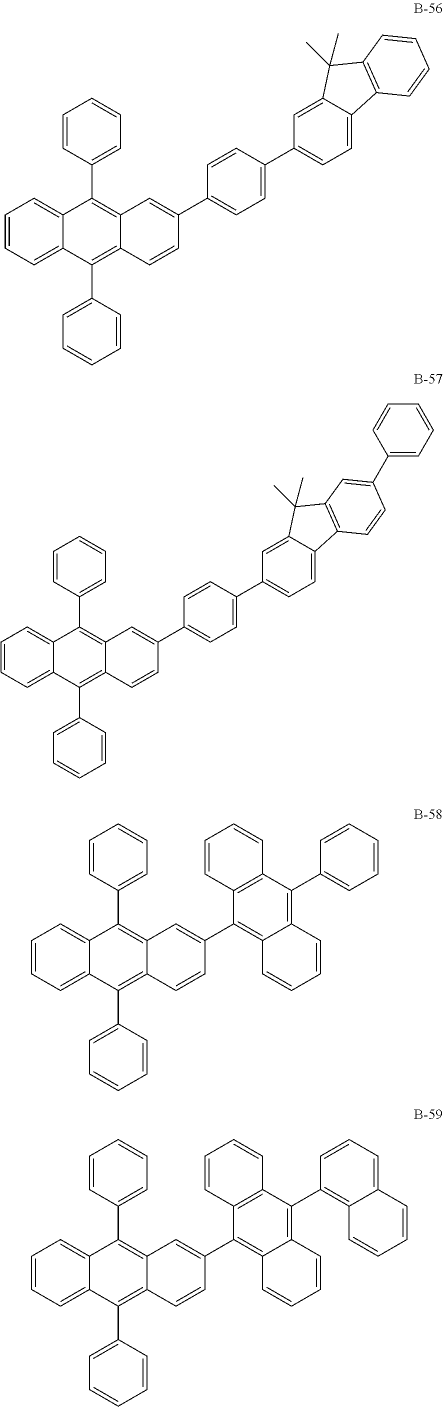

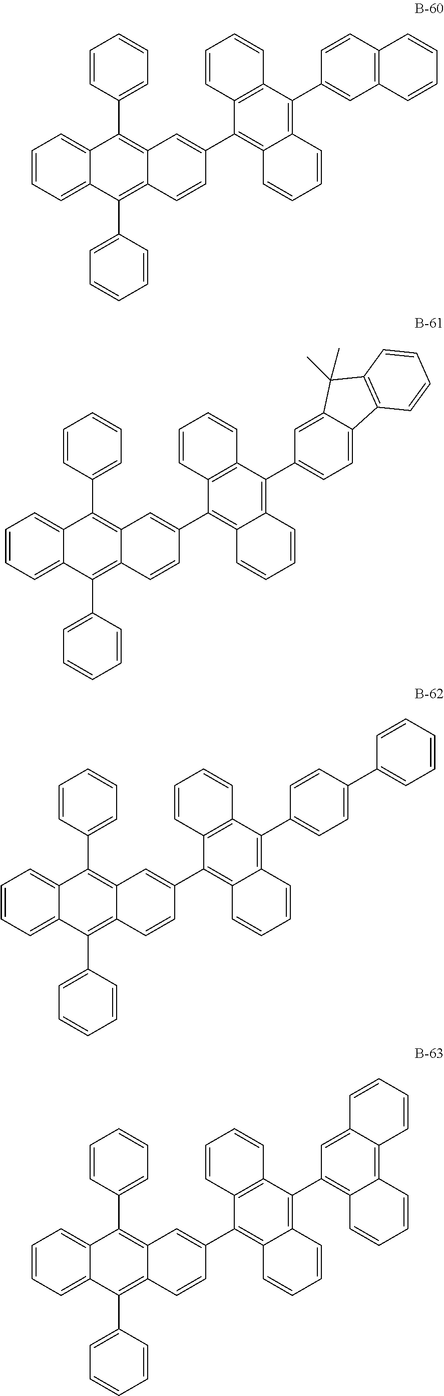

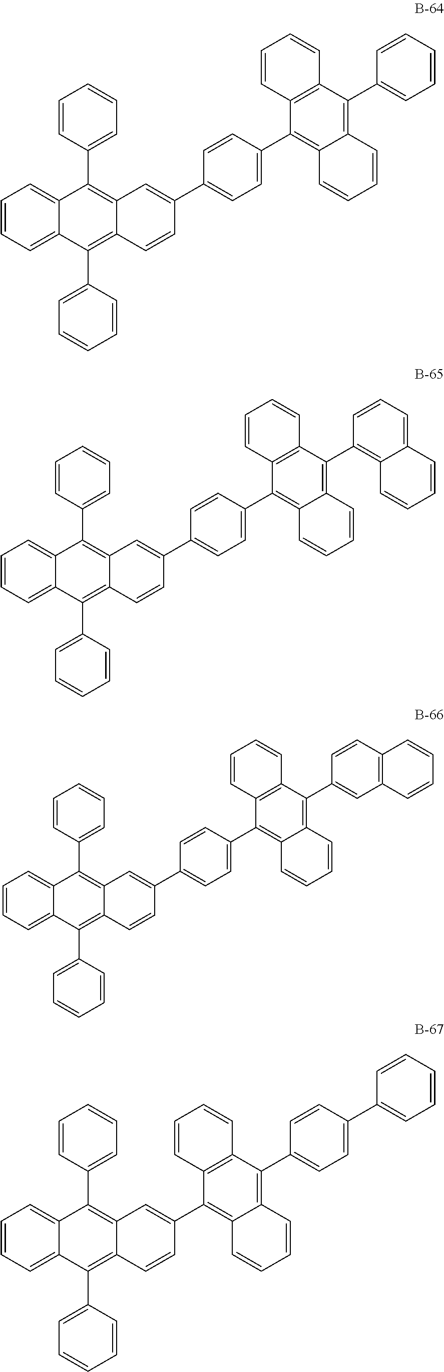

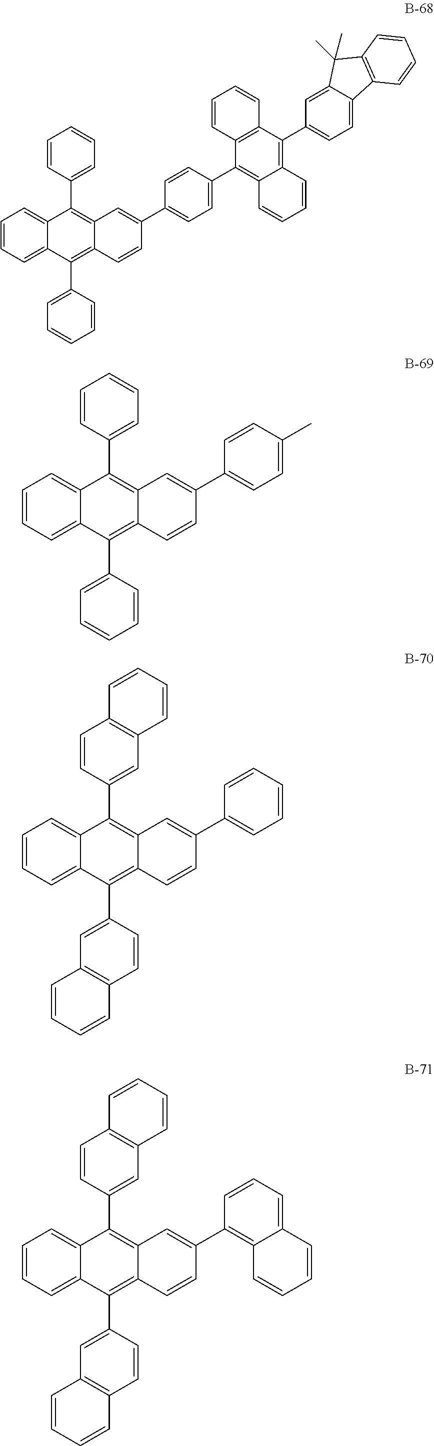

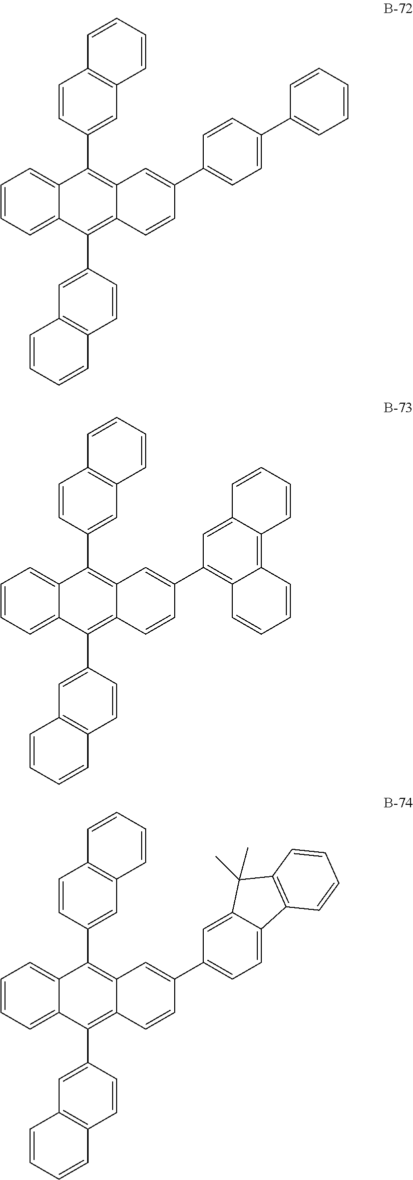

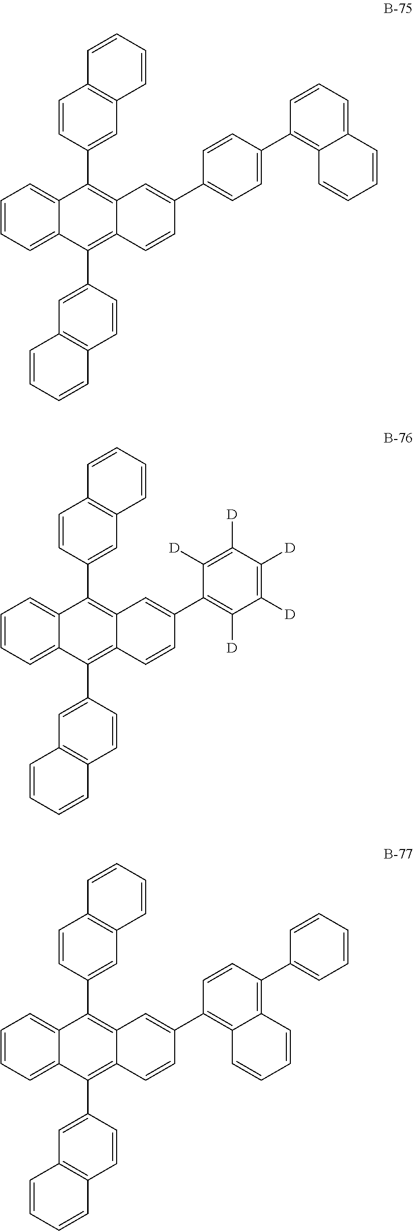

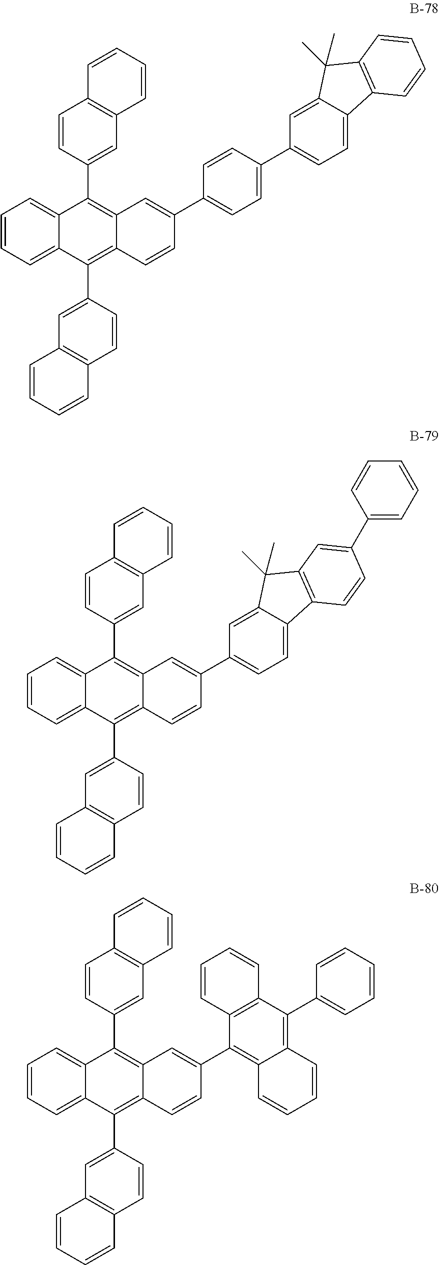

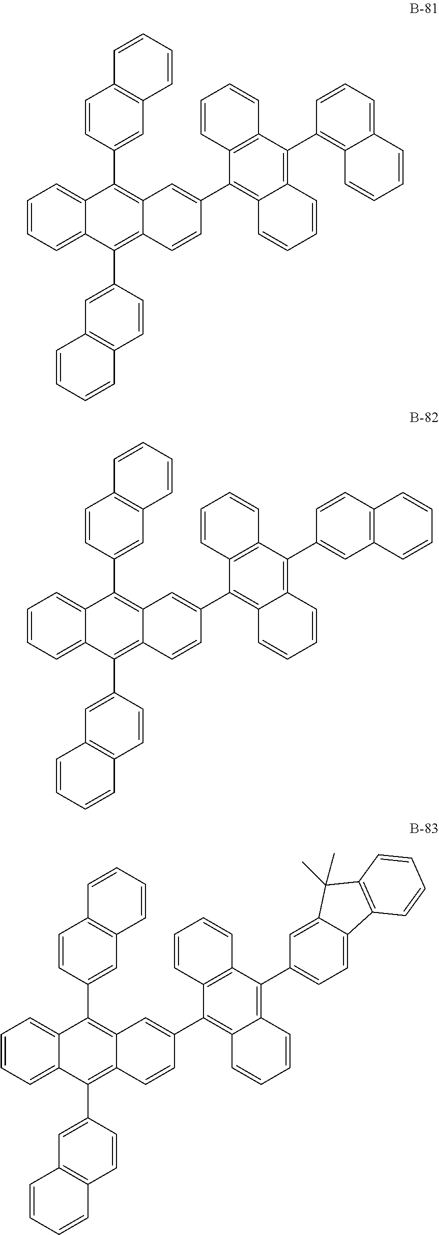

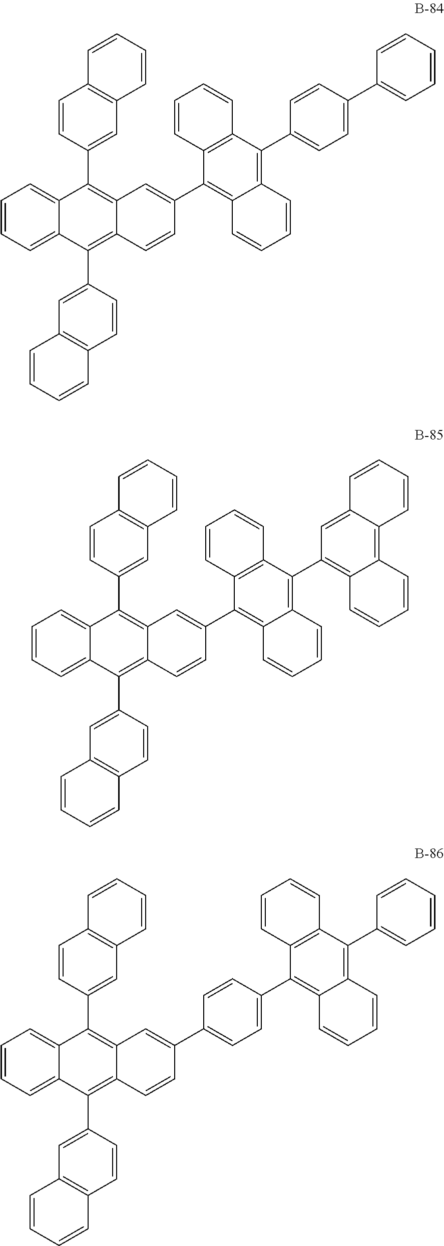

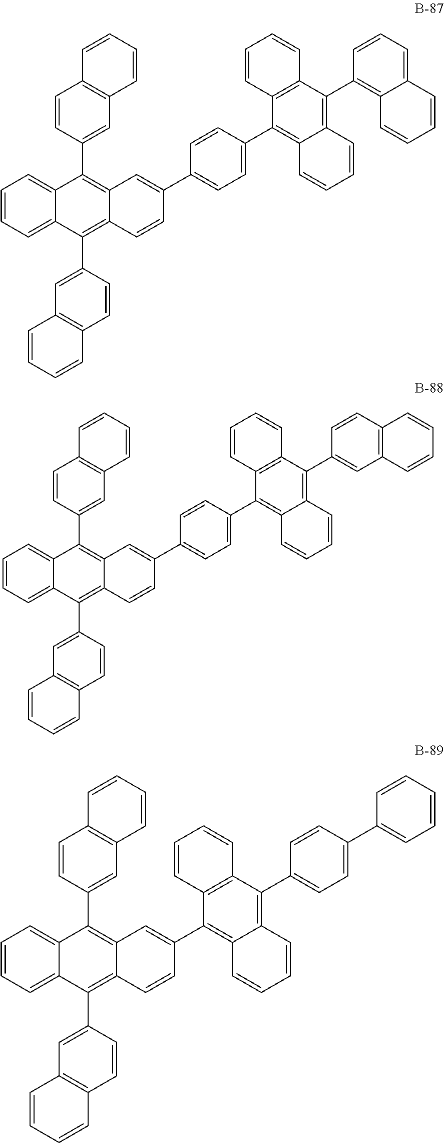

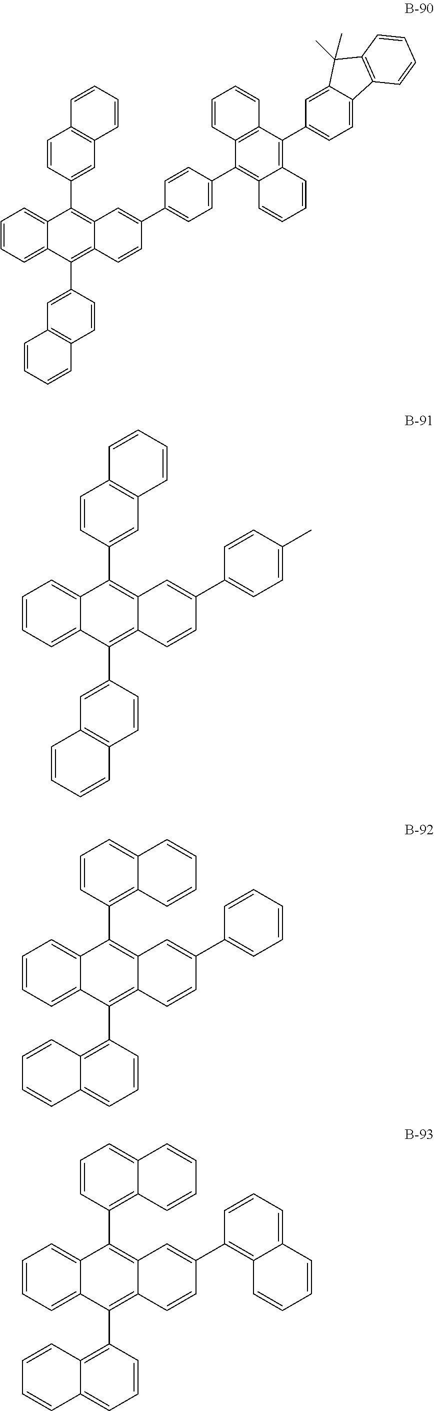

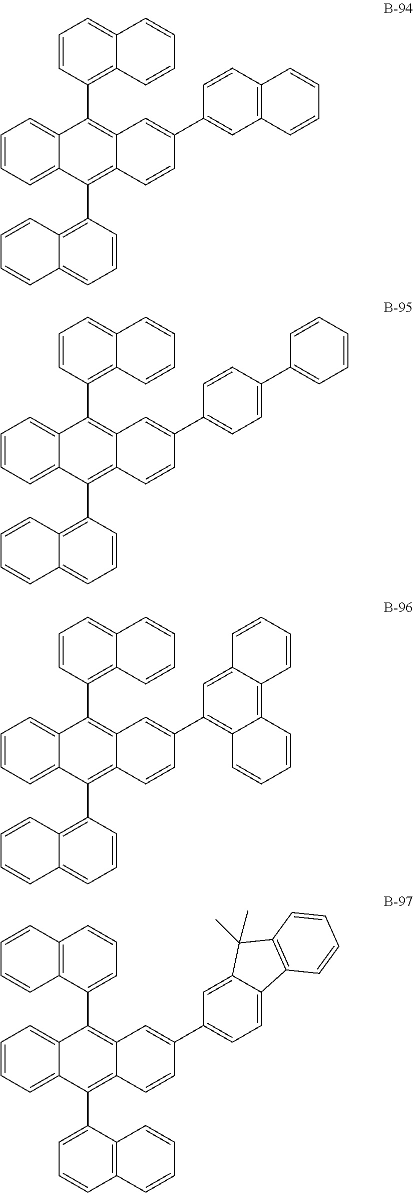

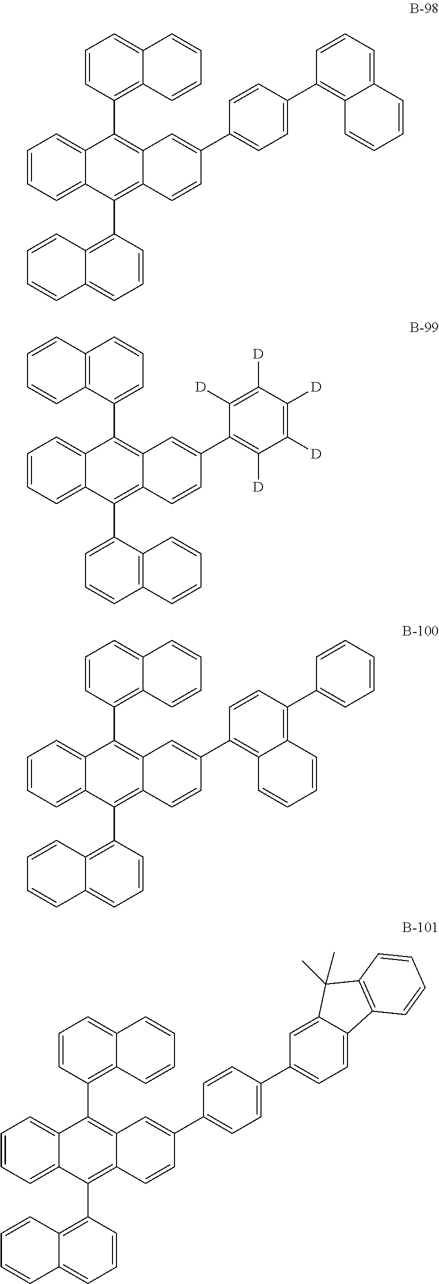

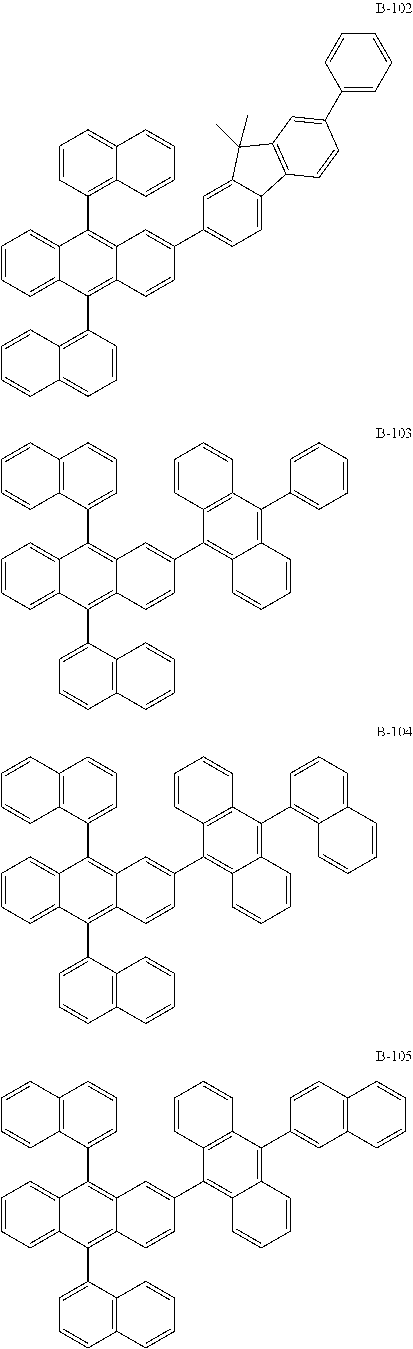

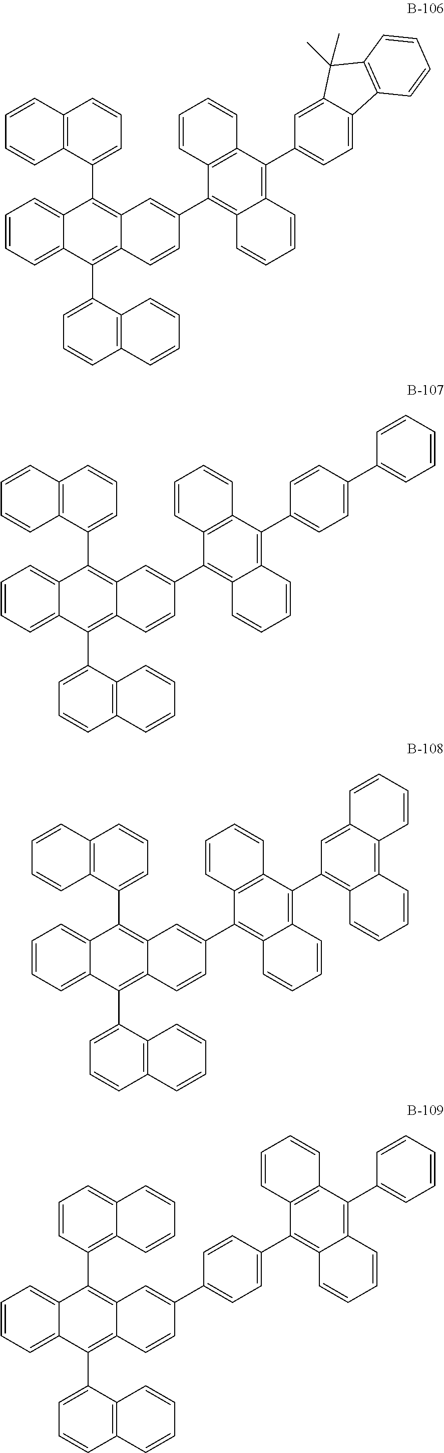

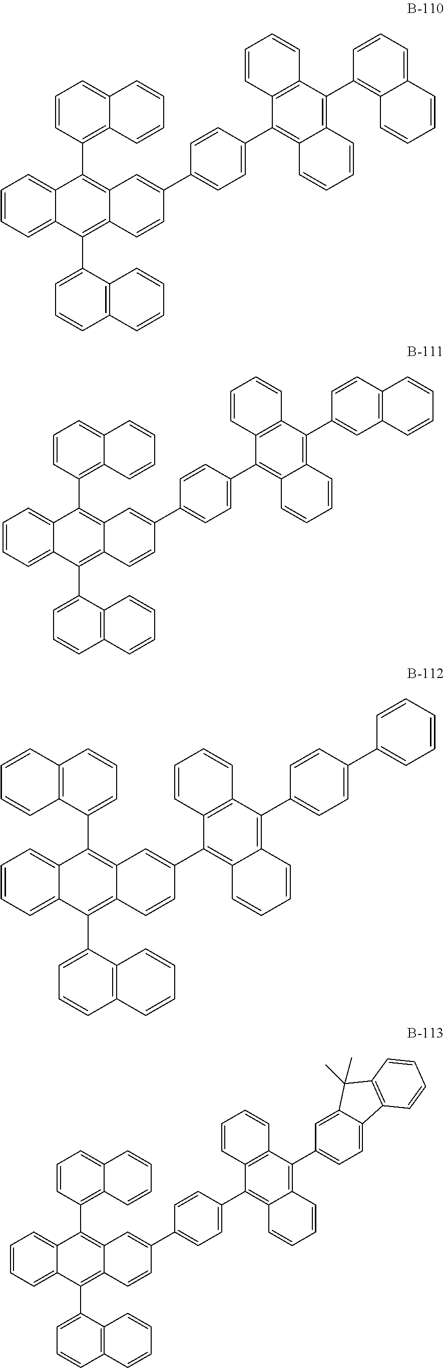

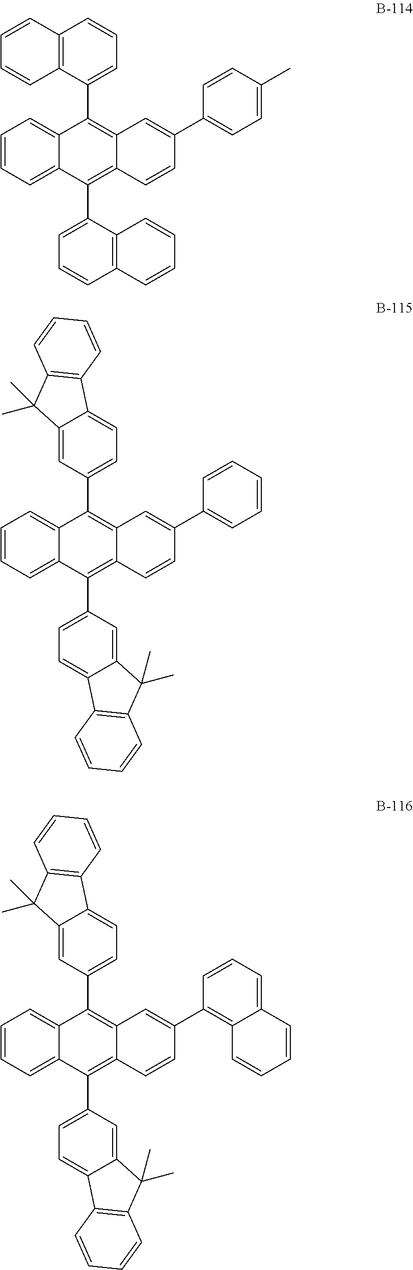

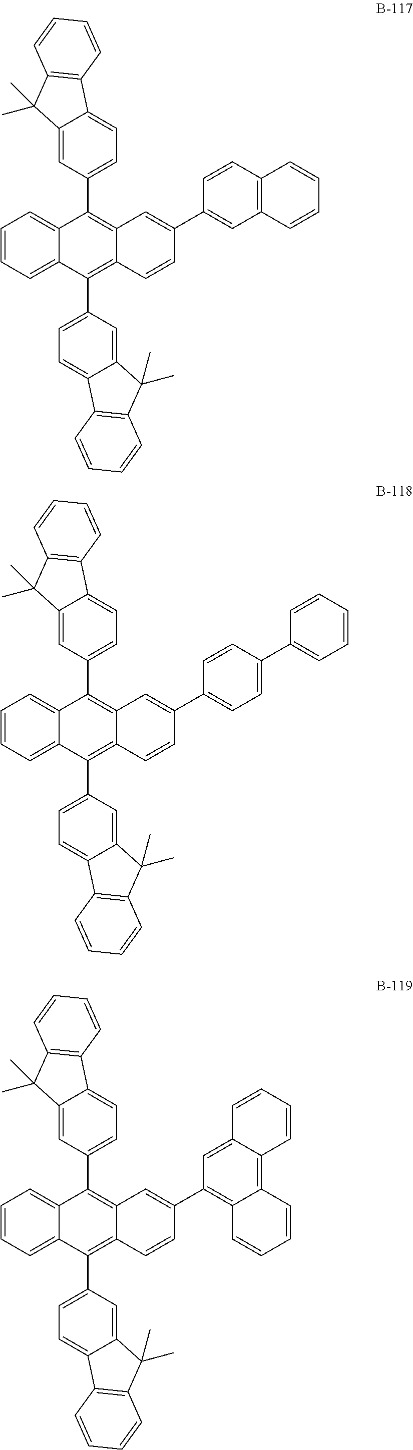

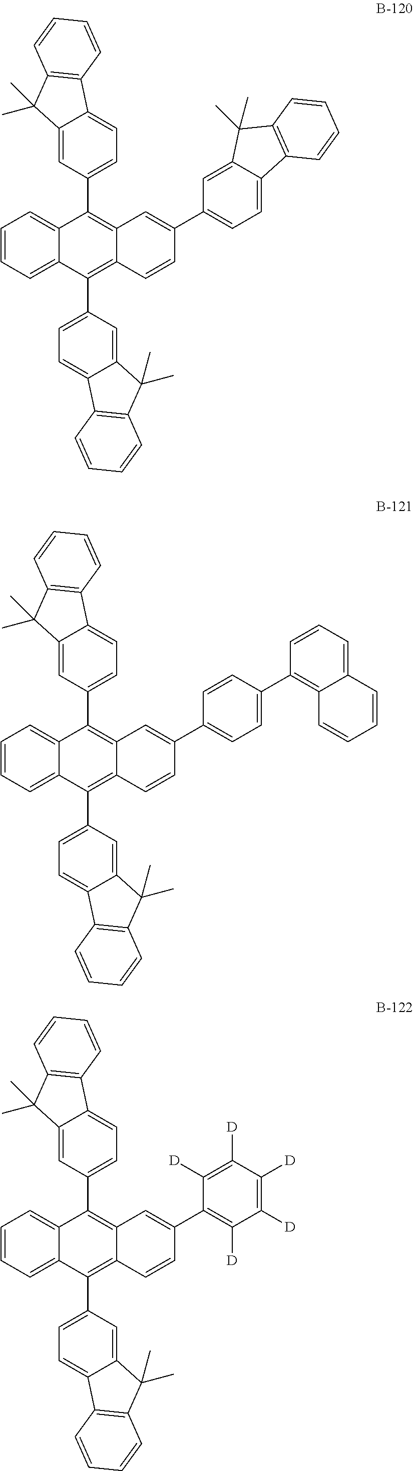

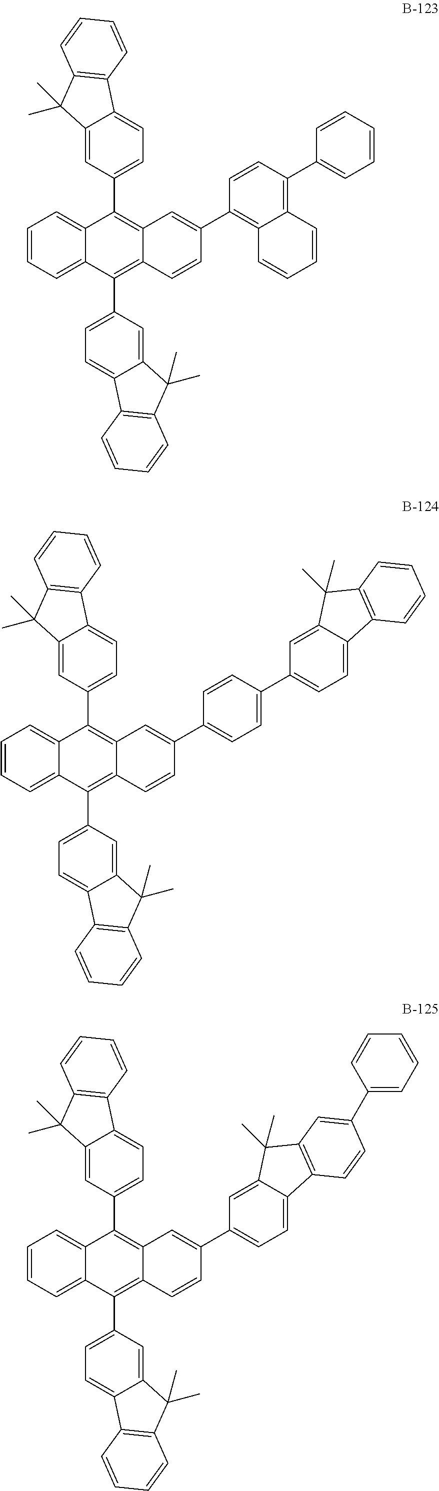

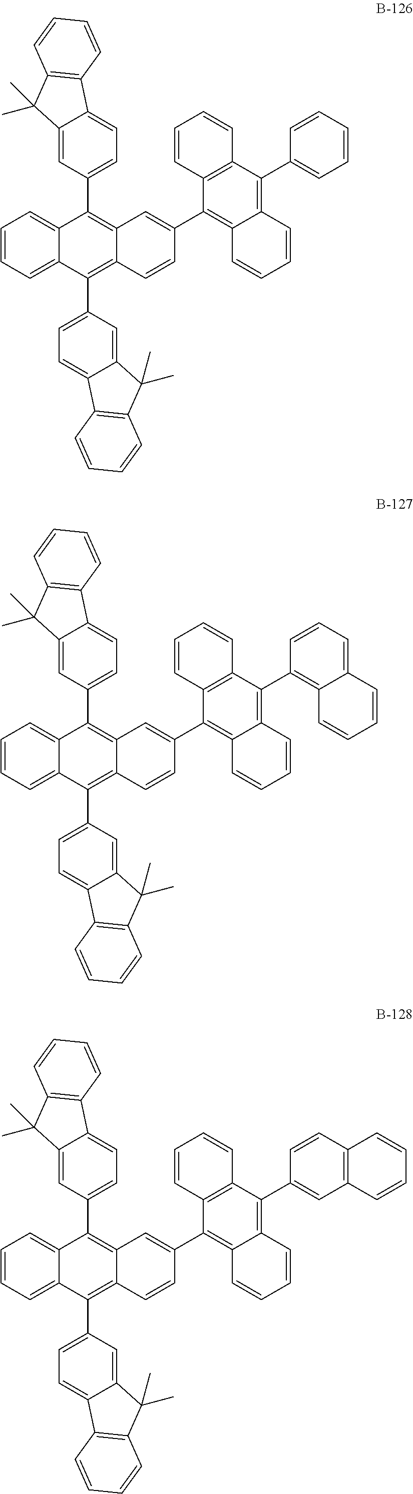

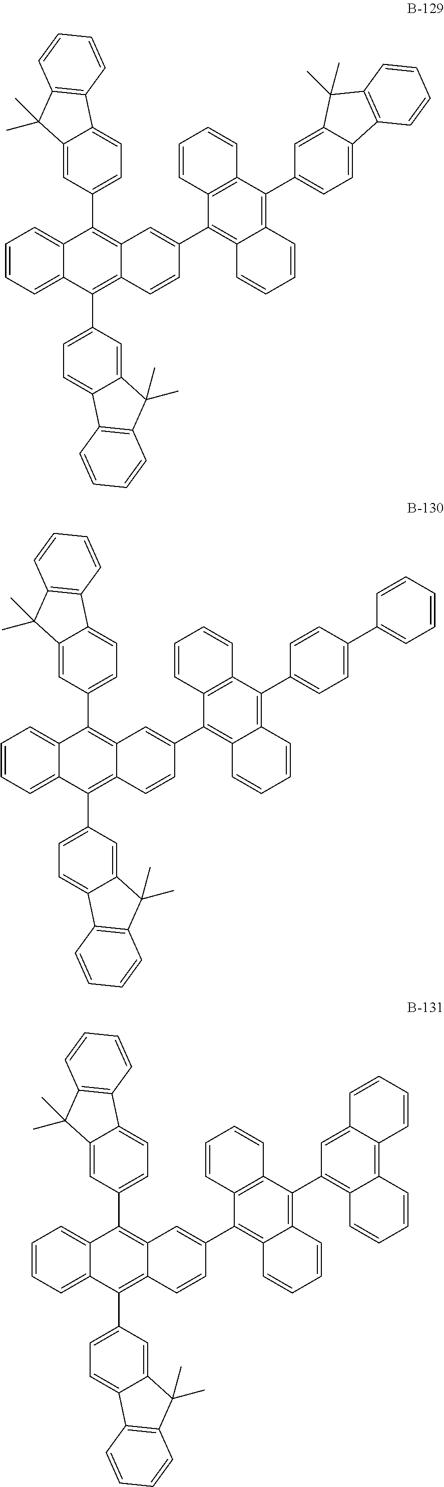

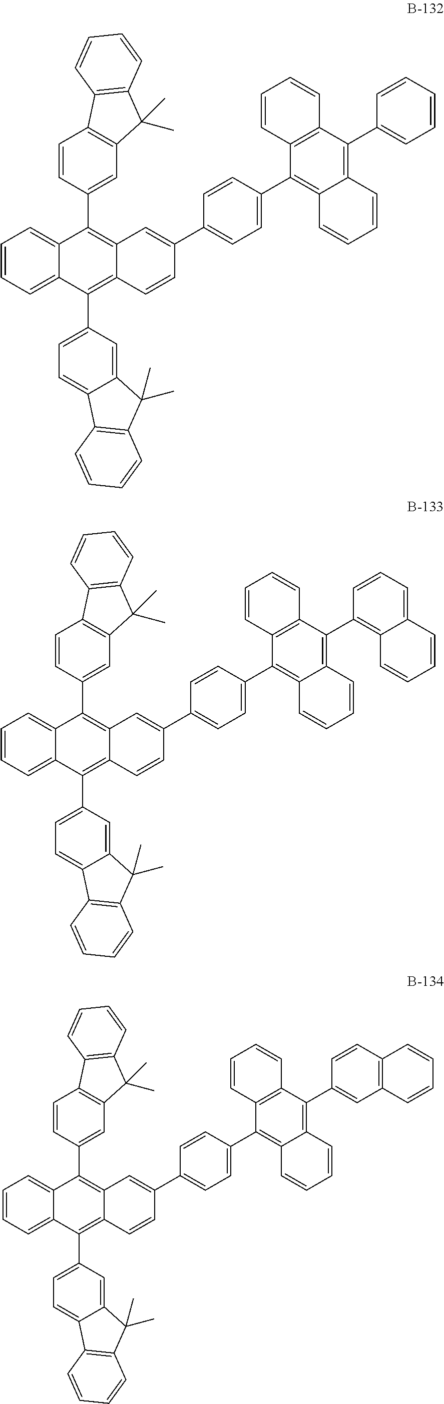

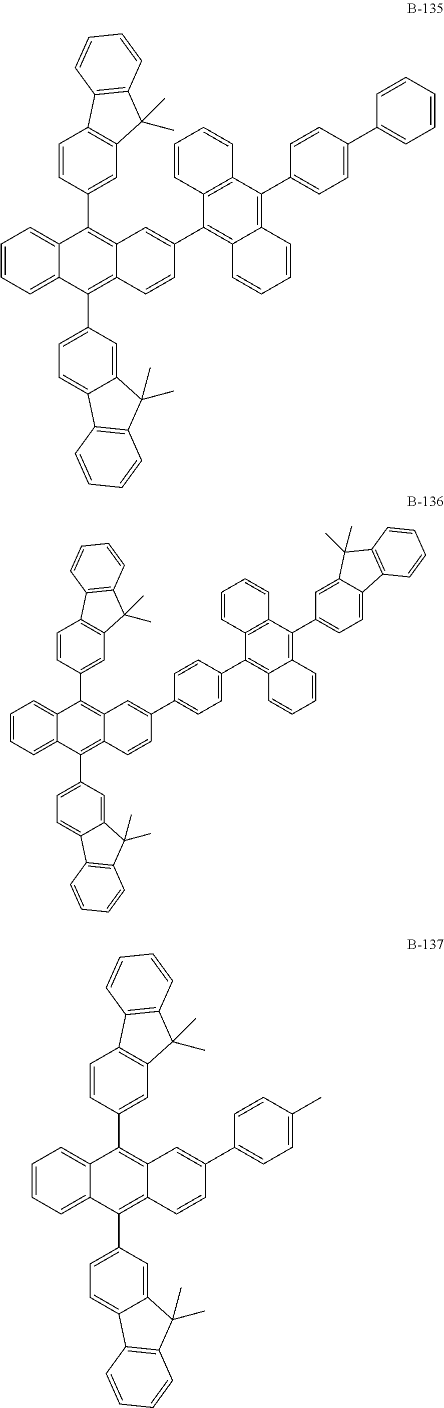

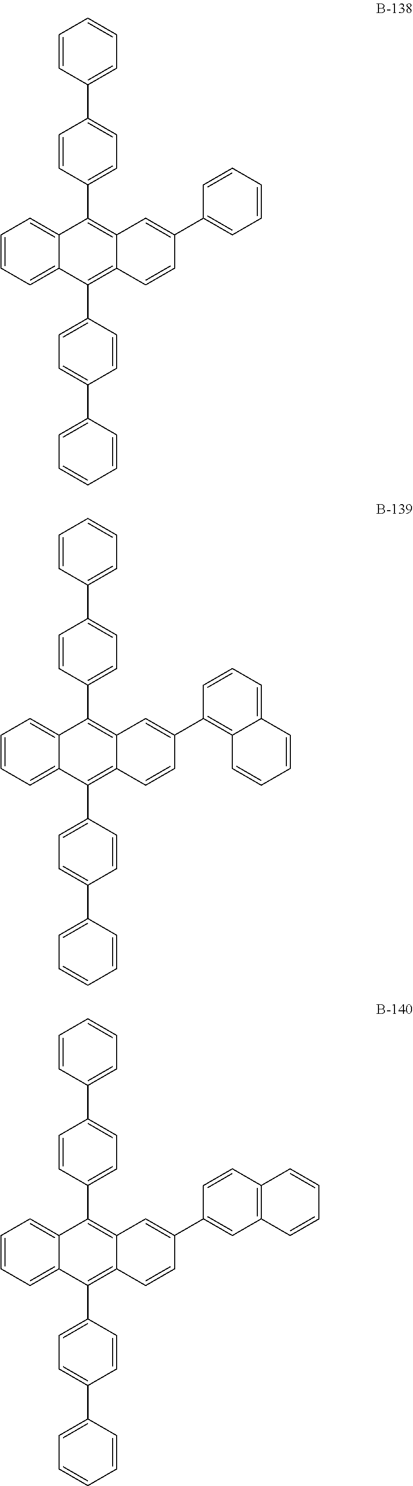

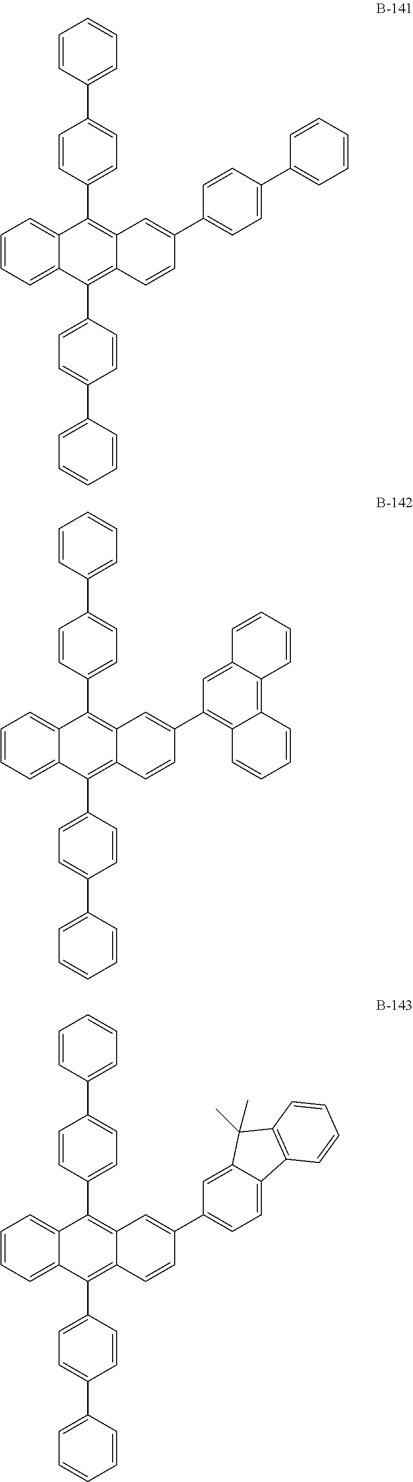

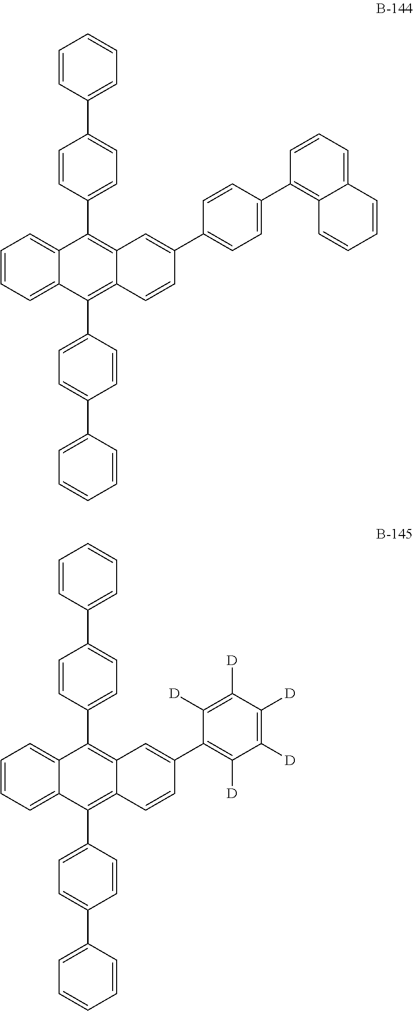

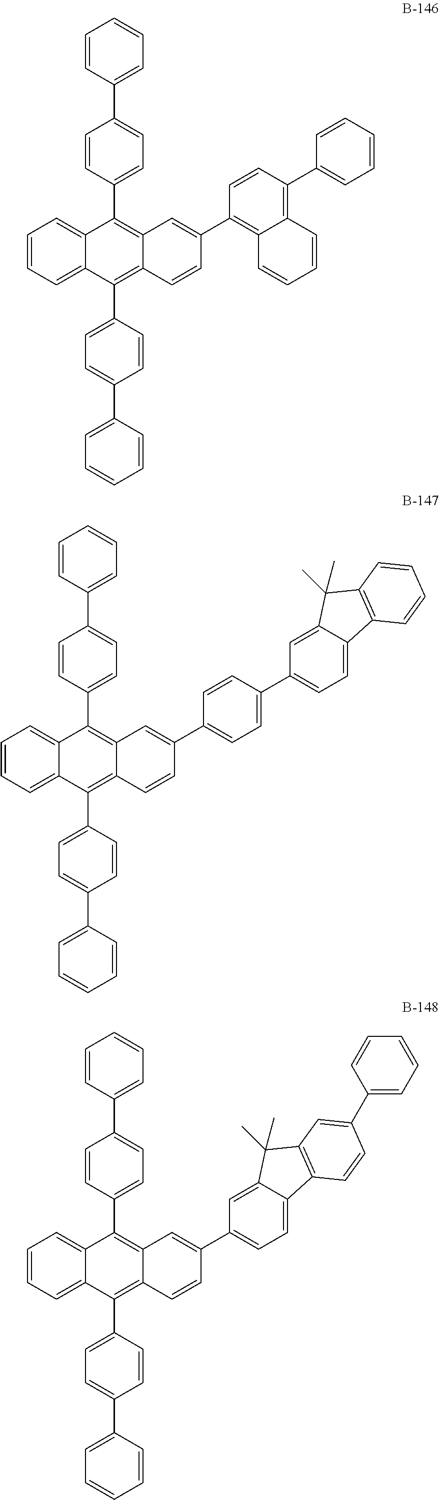

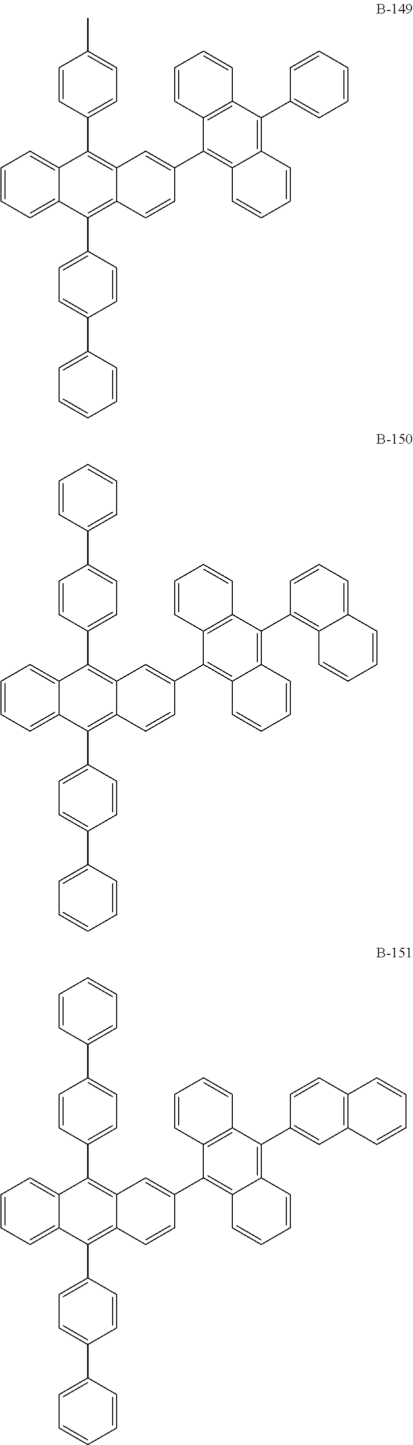

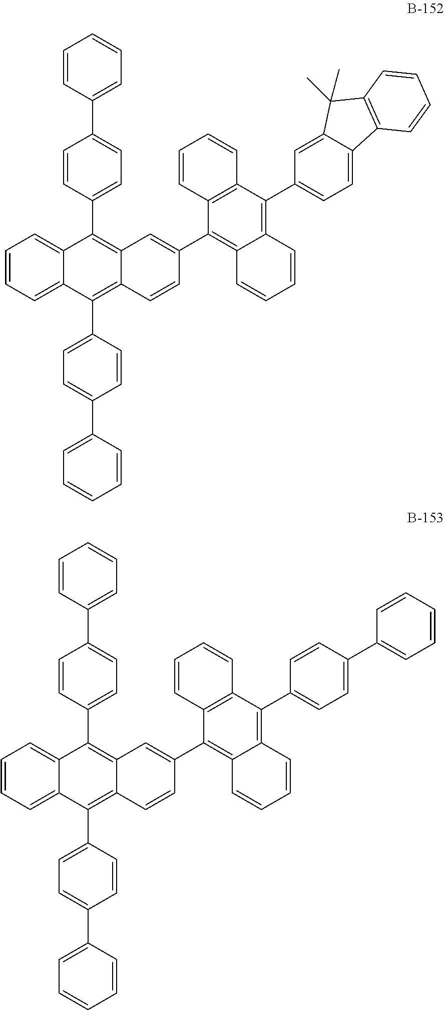

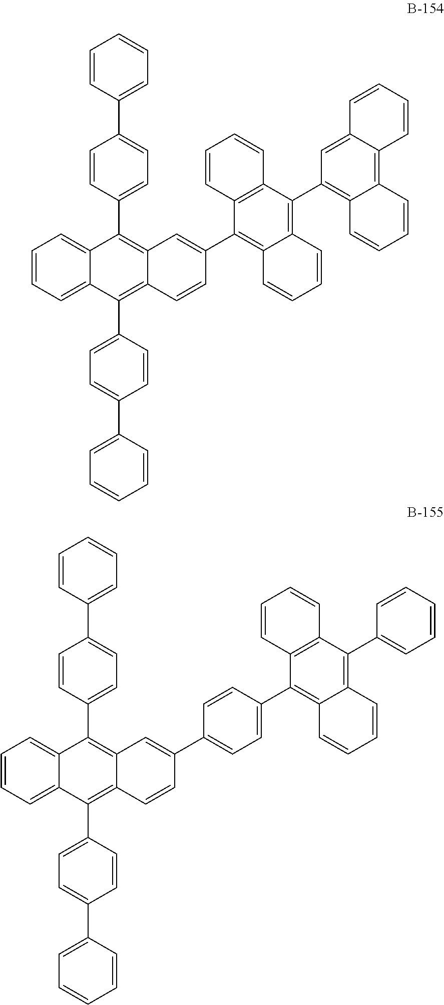

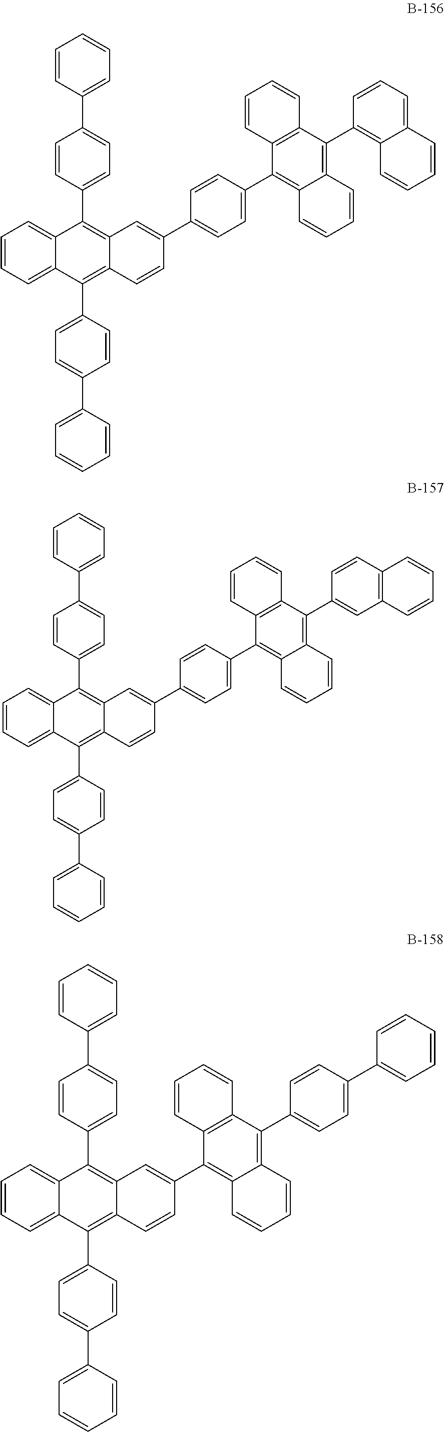

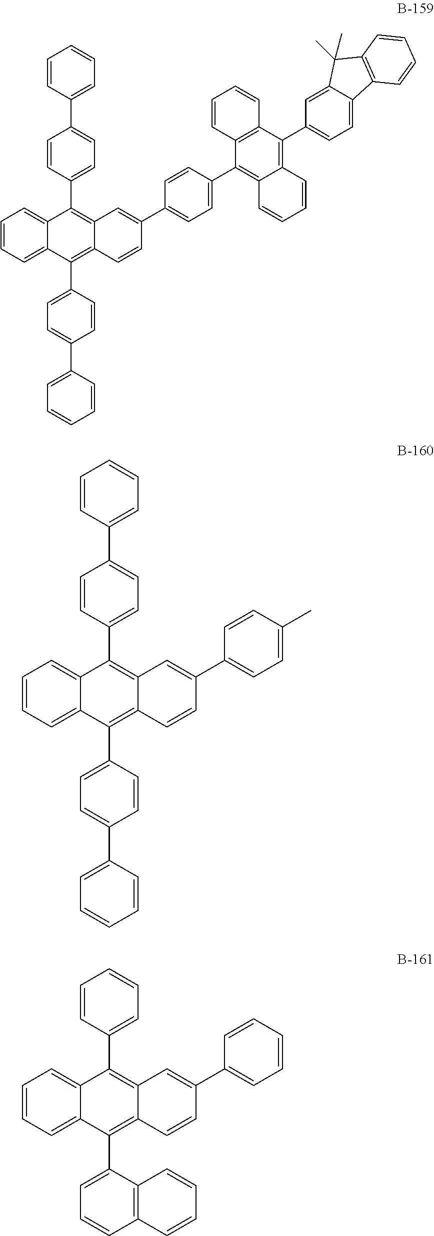

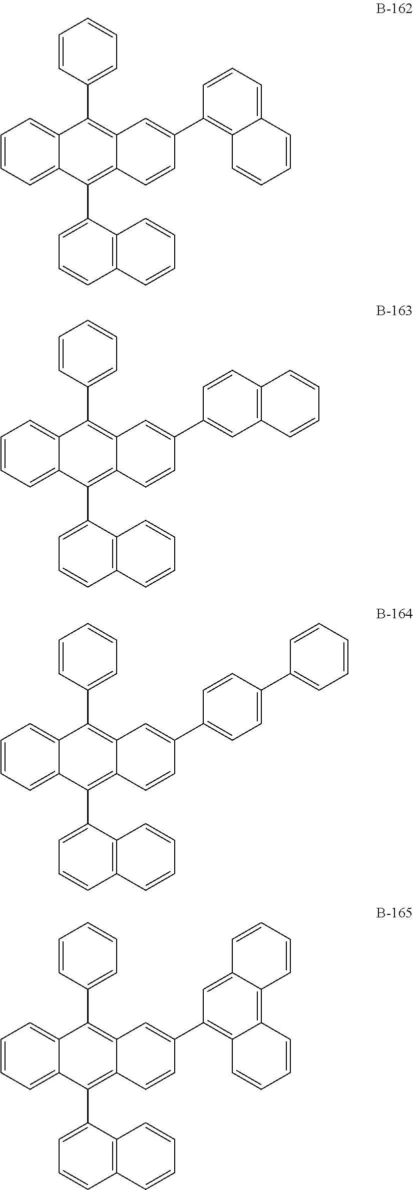

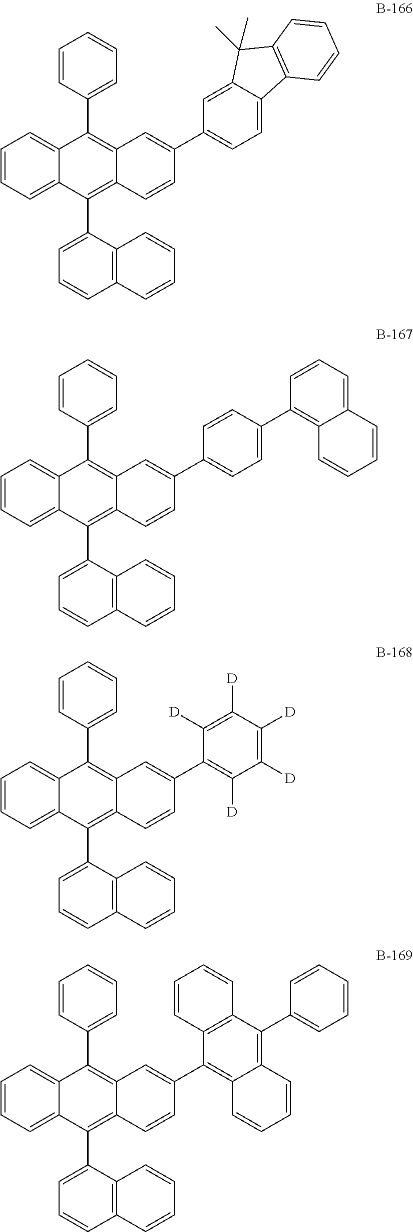

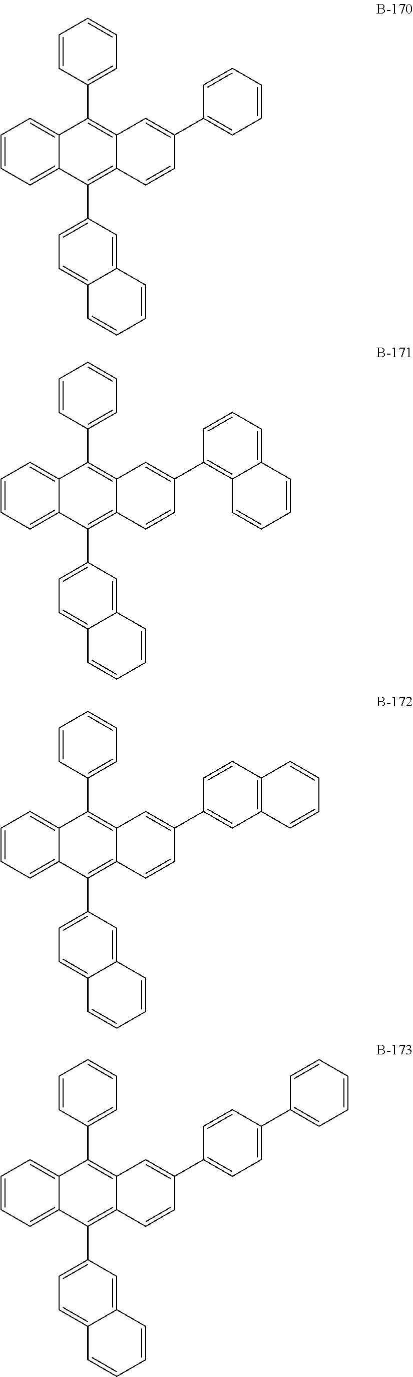

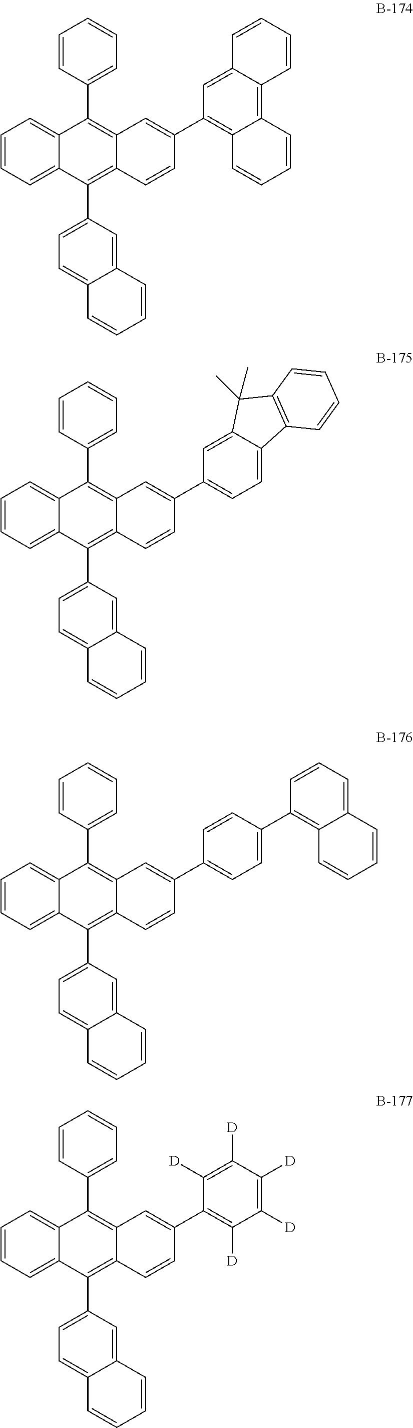

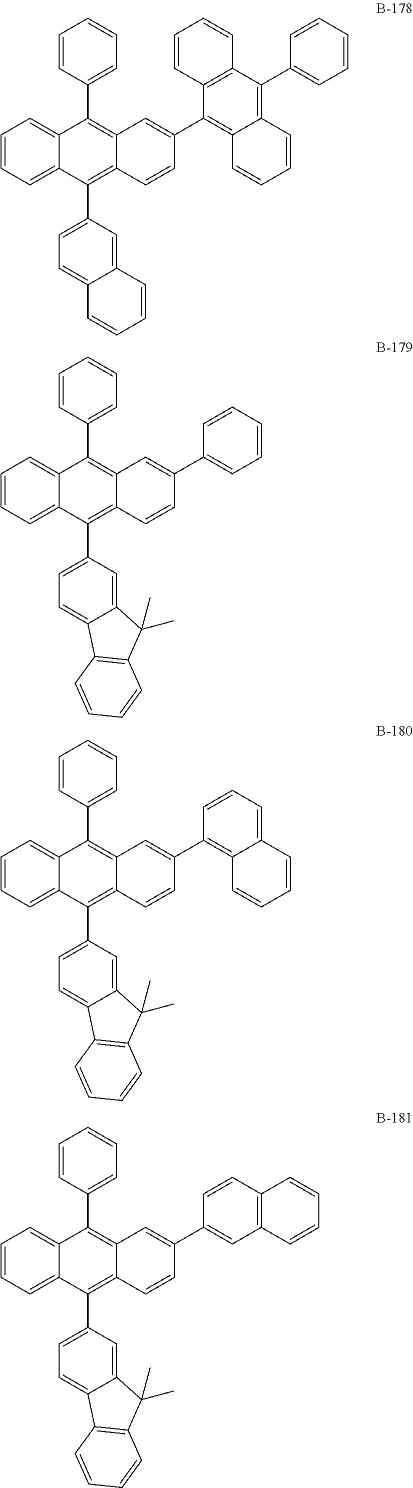

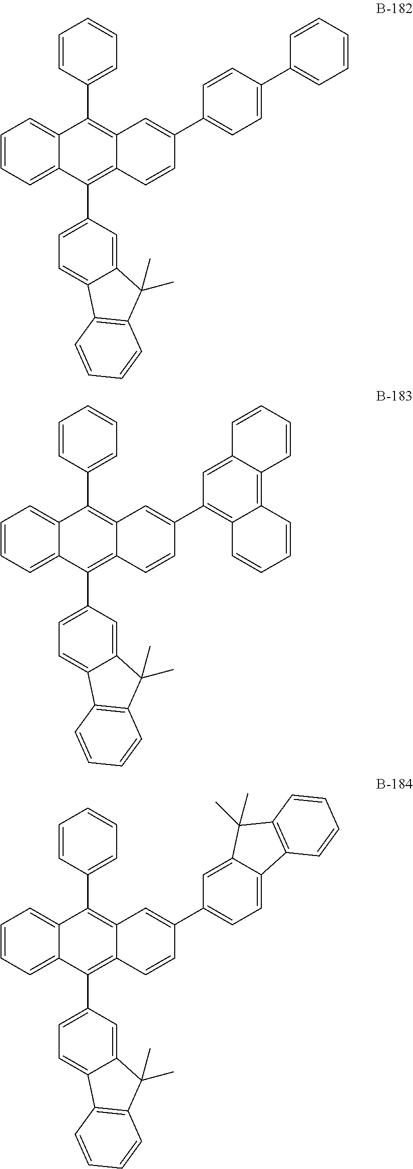

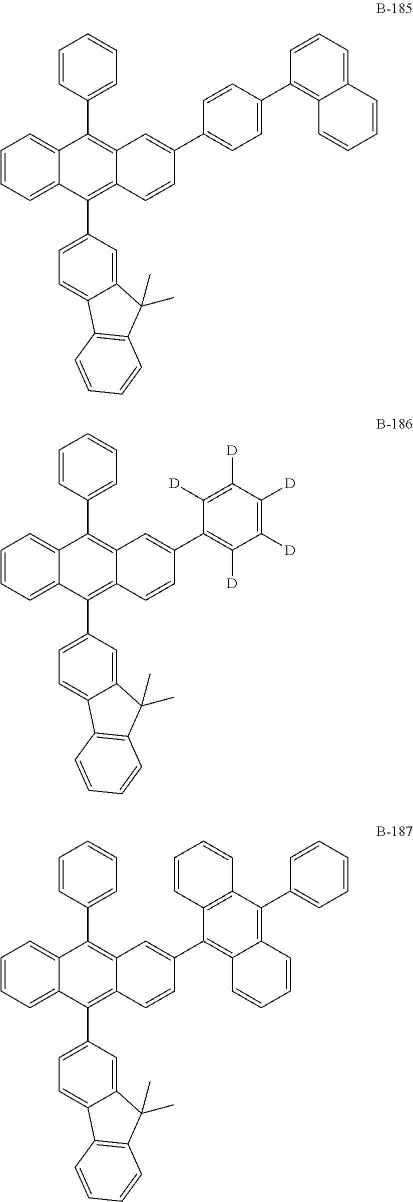

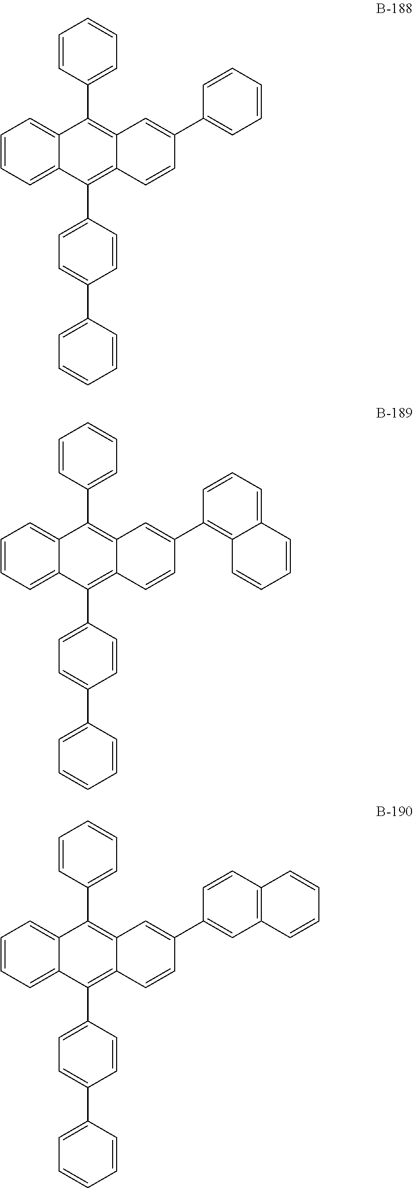

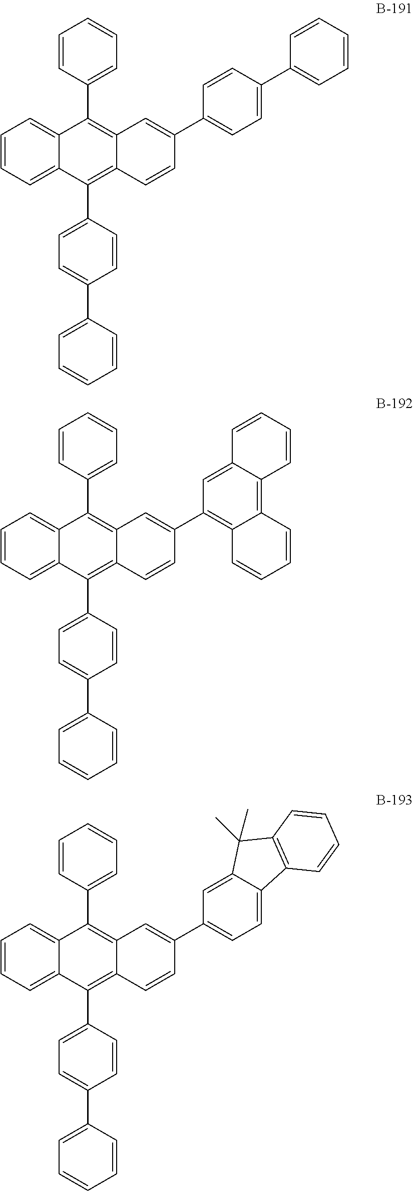

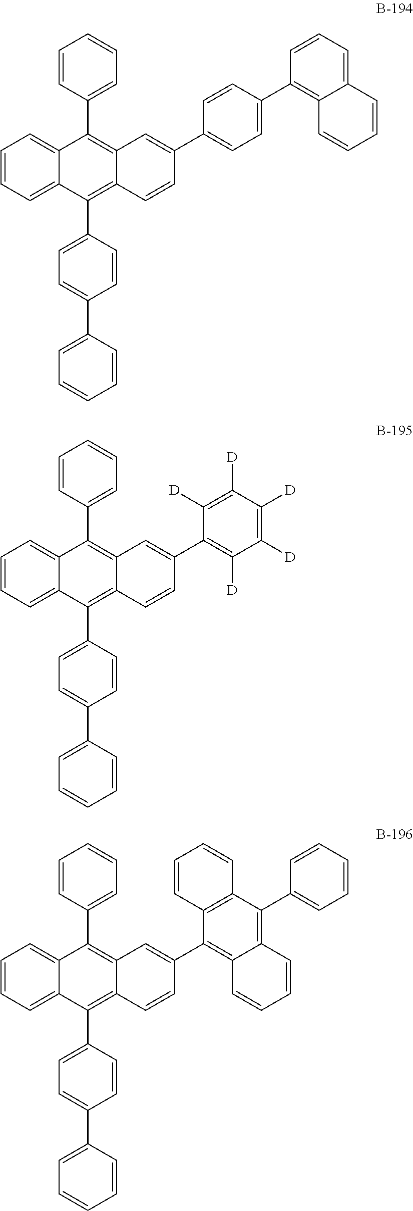

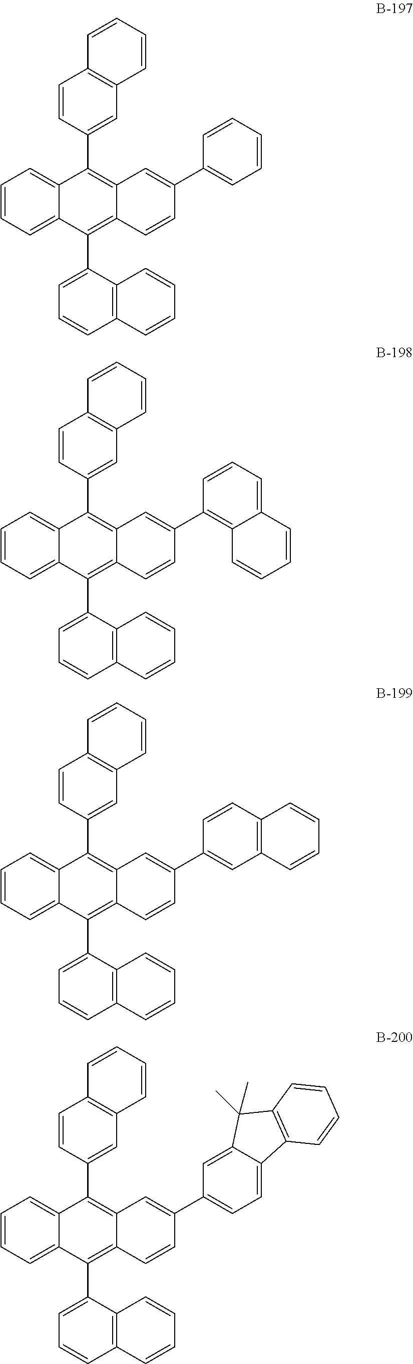

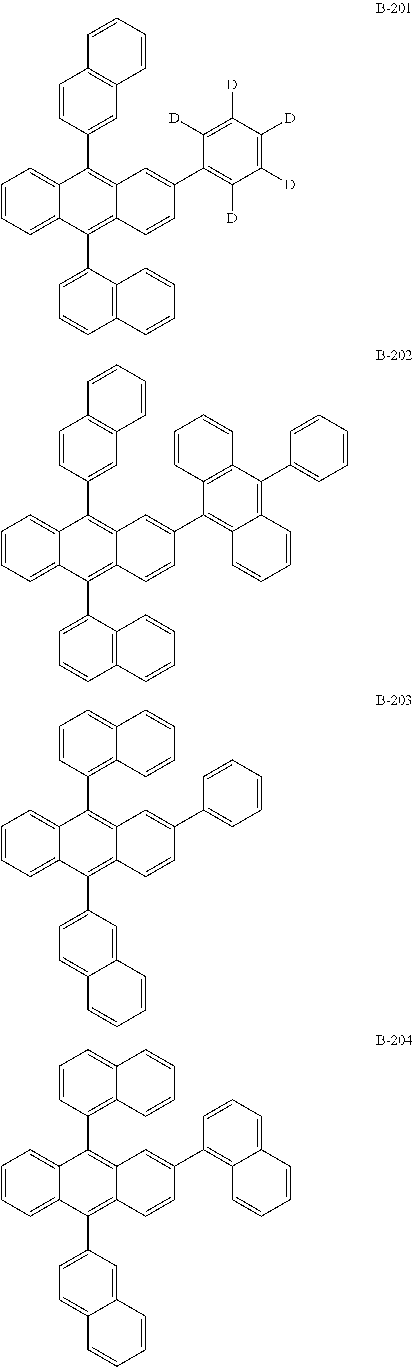

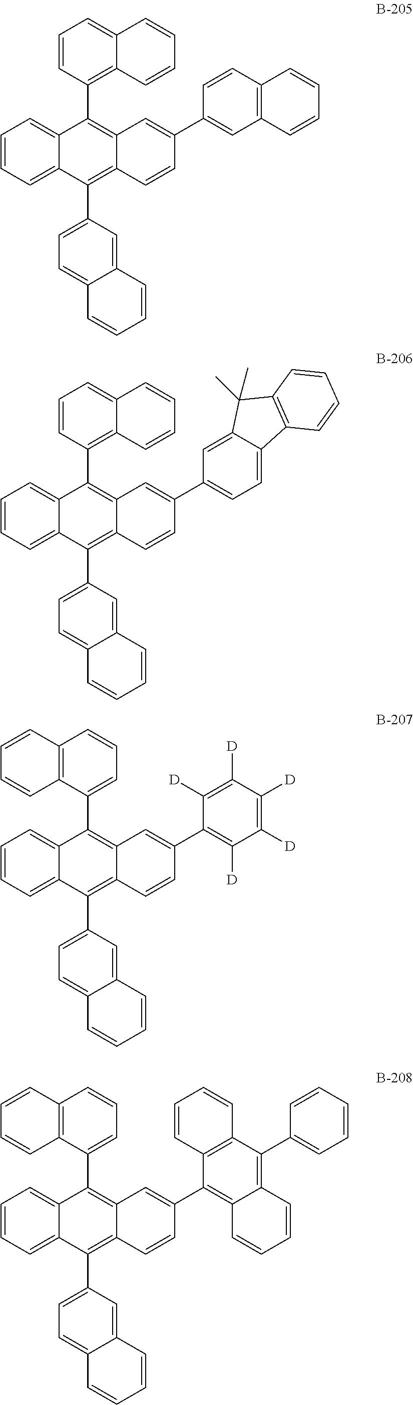

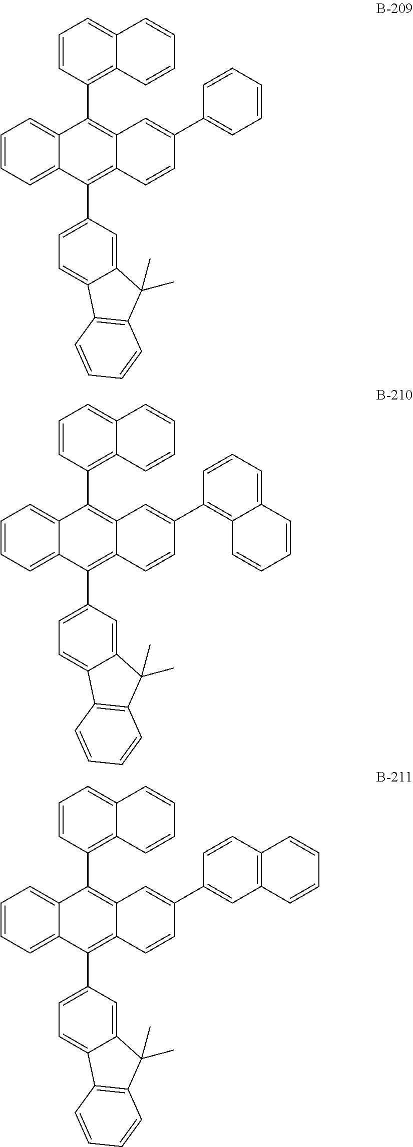

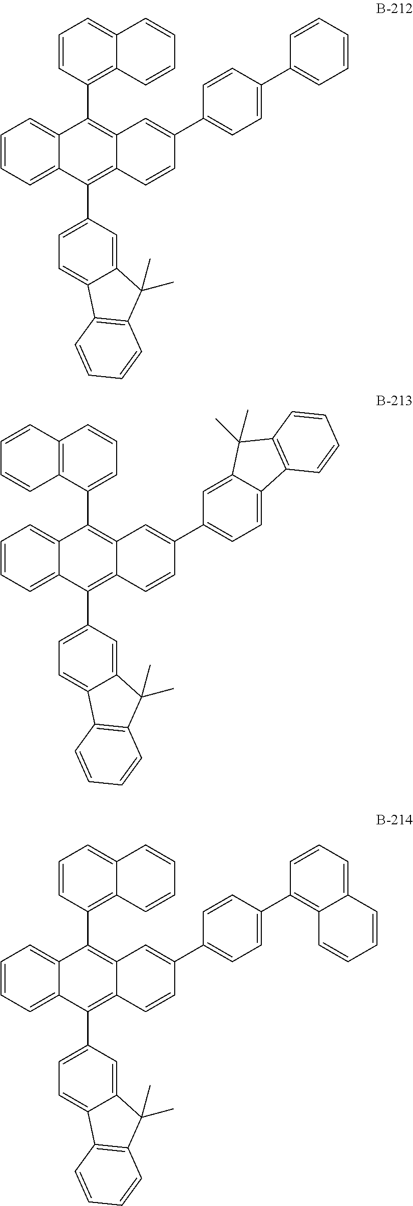

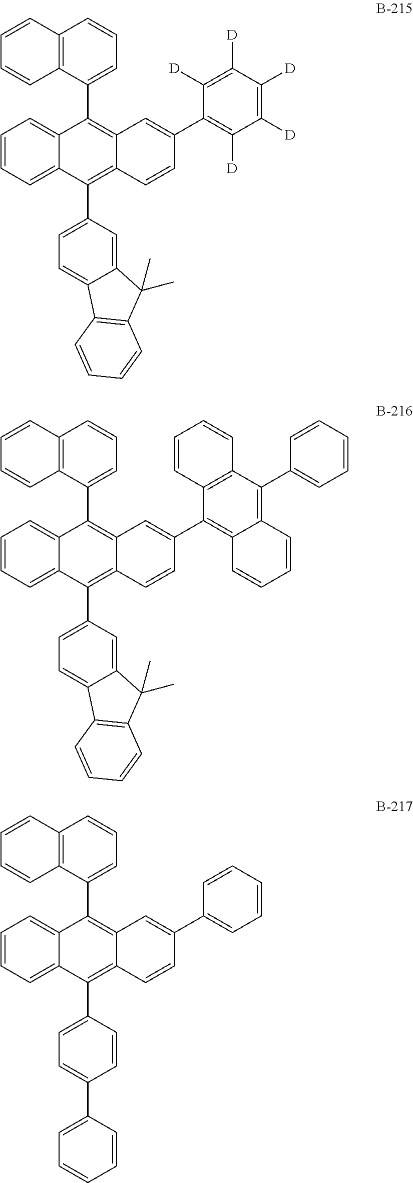

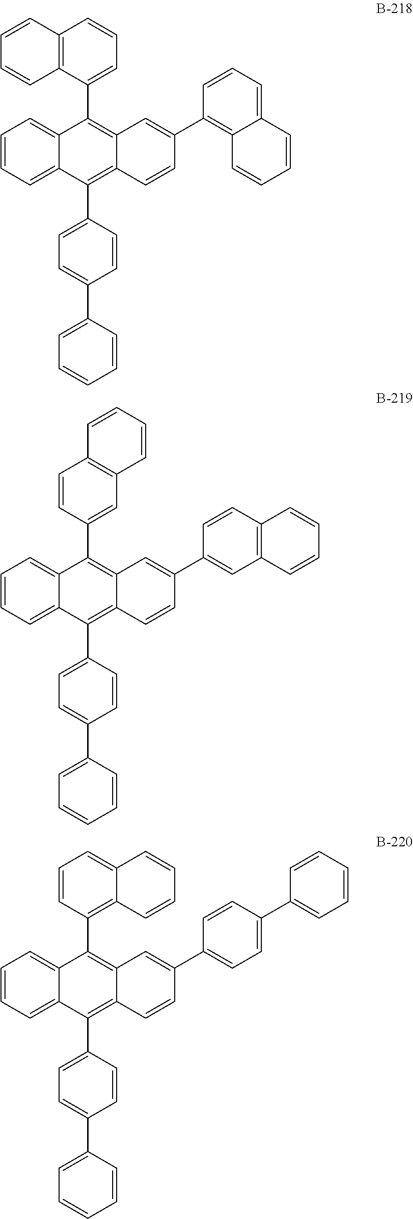

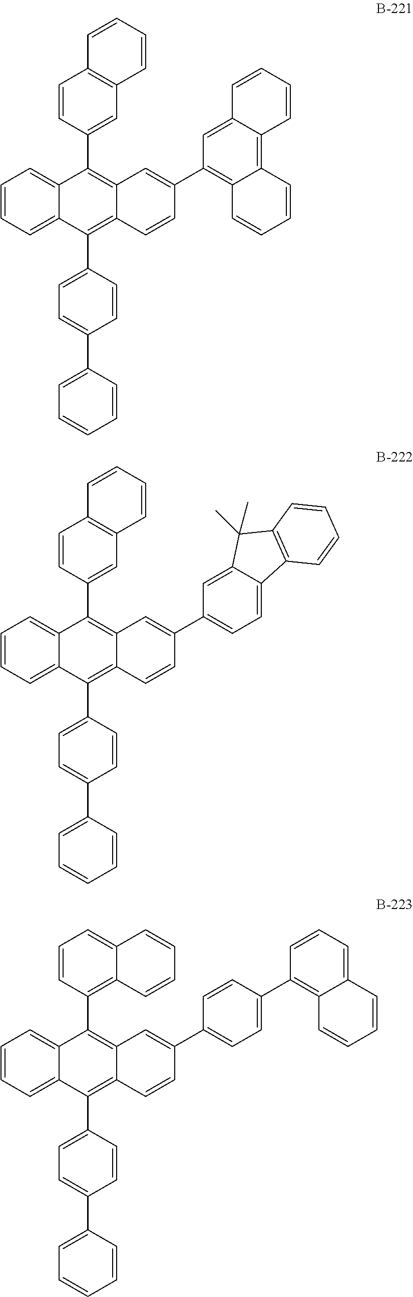

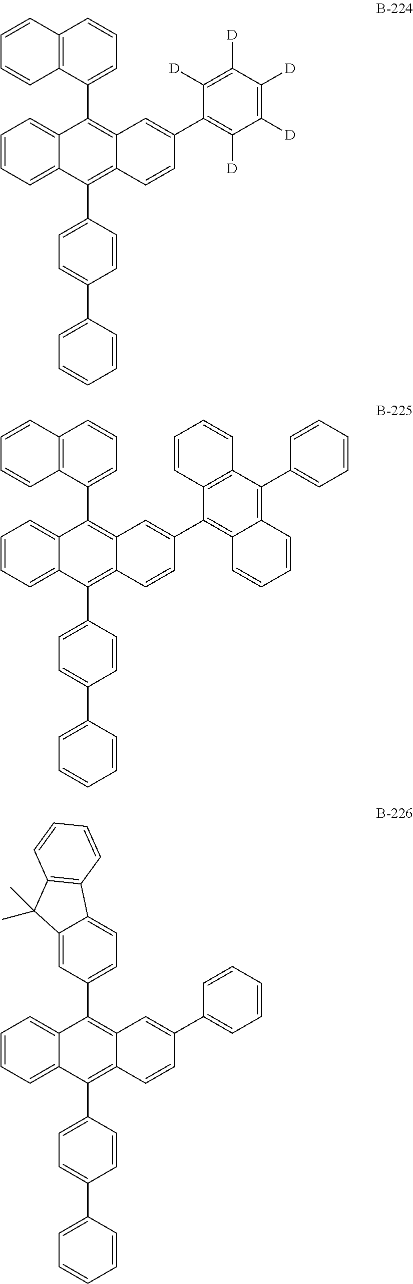

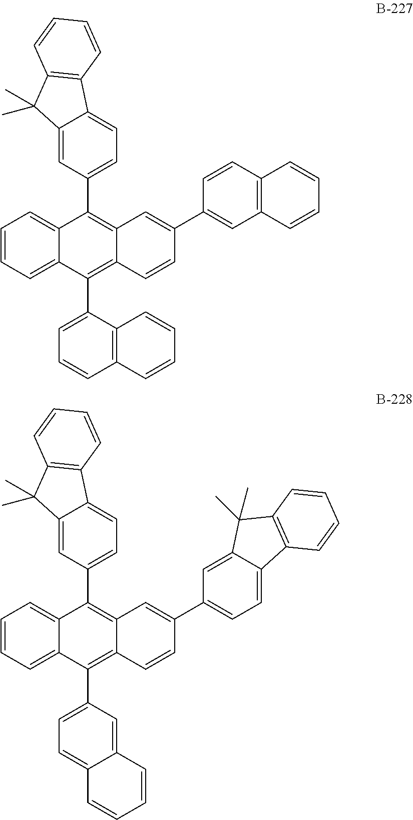

5. An organic electroluminescent device comprising a first electrode; a second electrode facing the first electrode; a light-emitting layer between the first electrode and the second electrode; and an electron transport zone and an electron buffering layer between the light-emitting layer and the second electrode, wherein the electron buffering layer comprises a compound represented by the following formula 1: 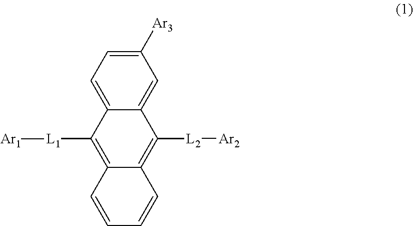
wherein
L₁ and L₂ each independently represent a single bond, a substituted or unsubstituted (C6-C30)arylene, or a substituted or unsubstituted (5- to 40-membered)heteroarylene;
Ar₁ to Arₐ each independently represent hydrogen, a substituted or unsubstituted (C6-C30)aryl, or a substituted or unsubstituted (5- to 40-membered)heteroaryl;
the heteroaryl(ene) contains at least one hetero atom selected from B, N, O, S, P(═O), Si, and P.

6. The organic electroluminescent device according to claim 5, wherein the light-emitting layer comprises a host compound and a dopant compound, and LUMO (Lowest Unoccupied Molecular Orbital) energy level of the electron buffering layer has ±0.2 eV based on LUMO energy level of the host compound.

7. The organic electroluminescent device according to claim 5, wherein the light-emitting layer comprises a host compound and a dopant compound, the host compound is the compound represented by the formula 1, and the host compound is the same as or different from the compound comprised in the the electron buffering layer.

8. The organic electroluminescent device according to claim 5, wherein the electron transport zone comprises an electron transport compound, a reductive dopant, or a combination thereof.

9. The organic electroluminescent device according to claim 8, wherein the electron transport compound is at least one selected from the group consisting of oxazole-based compounds, isoxazole-based compounds, triazole-based compounds, isothiazole-based compounds, oxadiazole-based compounds, thiadiazole-based compounds, perylene-based compounds, anthracene-based compounds, aluminum complexes, and gallium complexes, and the reductive dopant is at least one selected from the group consisting of alkali metals, alkali metal compounds, alkaline earth metals, rare-earth metals, and halides, oxides, and complexes thereof.

10. The organic electroluminescent device according to claim 9, wherein the electron transport compound is represented by the following formula 6: 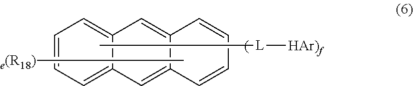
wherein
HAr is selected from the following formulae: 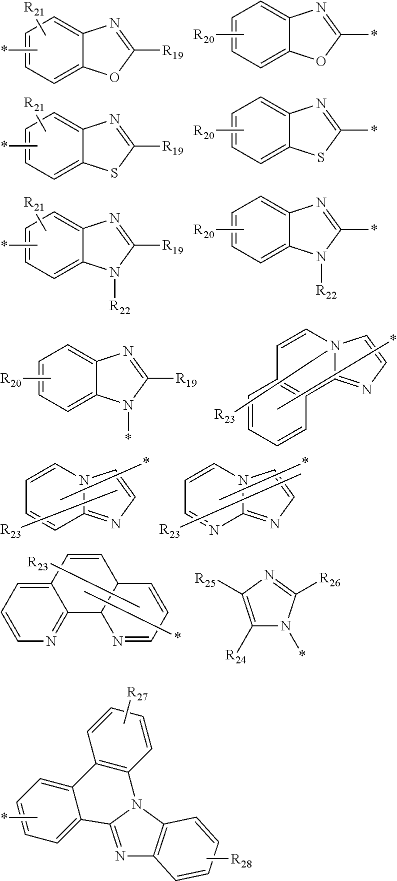
L represents a single bond, a substituted or unsubstituted (C6-C30)arylene, or a substituted or unsubstituted (5- to 30-membered)heteroarylene;
R₁₈ represents a substituted or unsubstituted a (C1-C30)alkyl, a substituted or unsubstituted (C6-C30)aryl, or a substituted or unsubstituted (5- to 30-membered)heteroaryl;
R₁₉ to R₂₈, each independently, represent hydrogen, deuterium, a halogen, a cyano, a carboxyl, a nitro, a hydroxyl, a substituted or unsubstituted (C1-C30)alkyl, a substituted or unsubstituted (C1-C30)alkoxy, a substituted or unsubstituted (C3-C30)cycloalkyl, a substituted or unsubstituted (C6-C30)aryl, or a substituted or unsubstituted (5- to 30-membered)heteroaryl; or are linked to an adjacent substituent(s) to form a substituted or unsubstituted, mono- or polycyclic, (C3-C30) alicyclic or aromatic ring, whose carbon atom(s) may be replaced with at least one hetero atom selected from nitrogen, oxygen, and sulfur;
e represents an integer of 0 to 3, where e is an integer of 2 or more, each of R₁₈ may be the same or different; and
f represents 1 or 2, where f is 2, each of (-L-HAr) may be the same or different.

11. The organic electroluminescent device according to claim 5, wherein the electron transport zone comprises an electron injection layer, an electron transport layer, or both of them.

12. The organic electroluminescent device according to claim 5, further comprising a hole injection layer, a hole transport layer, or both between the first electrode and the light-emitting layer.
